SEQUENCE LISTING (51 sequences)

SEQ ID NO: 1 - protein, 4 aa, Artificial Sequence
  Description of Artificial Sequence: Synthetic peptide
WMEW

SEQ ID NO: 2 - protein, 34 aa, Artificial Sequence
  Description of Artificial Sequence: Synthetic peptide
WMEWDREINNYTSLIHSLIEESQNQQEKNEQELL

SEQ ID NO: 3 - protein, 36 aa, Artificial Sequence
  Description of Artificial Sequence: Synthetic peptide
YTSLIHSLIEESQNQQEKNEQELLELDKWASLWNWF

SEQ ID NO: 4 - protein, 38 aa, Artificial Sequence
  Description of Artificial Sequence: Synthetic peptide
NNLLRAIEAQQHLLQLTVWQIKQLQARILAVERYLKDQ

SEQ ID NO: 5 - protein, 34 aa, Artificial Sequence; identical to SEQ ID NO: 2
  Description of Artificial Sequence: Synthetic peptide

SEQ ID NO: 6 - protein, 34 aa, Artificial Sequence
  Description of Artificial Sequence: Synthetic peptide
WMEWDREINNYTSLIHSLIEESQNQQERNEQELK

SEQ ID NO: 7 - protein, 34 aa, Artificial Sequence
  Description of Artificial Sequence: Synthetic peptide
WMEWDREINNYTSLIHSLIEESQNQQERNEQEKL

SEQ ID NO: 8 - protein, 34 aa, Artificial Sequence
  Description of Artificial Sequence: Synthetic peptide
WMEWDREINNYTSLIHSLIEESQNQQERNEQKLL

SEQ ID NO: 9 - protein, 34 aa, Artificial Sequence
  Description of Artificial Sequence: Synthetic peptide
WMEWDREINNYTSLIHSLIEESQNQQERNEKELL

SEQ ID NO: 10 - protein, 34 aa, Artificial Sequence
  Description of Artificial Sequence: Synthetic peptide
WMEWDREINNYTSLIHSLIEESQNQQERNKQELL

SEQ ID NO: 11 - protein, 34 aa, Artificial Sequence
  Description of Artificial Sequence: Synthetic peptide
WMEWDREINNYTSLIHSLIEESQNQQERKEQELL

SEQ ID NO: 12 - protein, 35 aa, Artificial Sequence
  Description of Artificial Sequence: Synthetic peptide
WMEWDREINNYTSLIHSLIEESQNQQERNEQELLK

SEQ ID NO: 13 - protein, 35 aa, Artificial Sequence
  Description of Artificial Sequence: Synthetic peptide
WMEWDREINNYTSLIHSLIEESQNQQEKNEQELLK

SEQ ID NO: 14 - protein, 36 aa, Artificial Sequence; identical to SEQ ID NO: 3
  Description of Artificial Sequence: Synthetic peptide

SEQ ID NO: 15 - protein, 36 aa, Artificial Sequence
  Description of Artificial Sequence: Synthetic peptide
YTNTIYTLLEESQNQQEKNEQELLELDKWASLWNWF

SEQ ID NO: 16 - protein, 36 aa, Artificial Sequence
  Description of Artificial Sequence: Synthetic peptide
YTGIIYNLLEESQNQQEKNEQELLELDKWANLWNWF

SEQ ID NO: 17 - protein, 36 aa, Artificial Sequence
  Description of Artificial Sequence: Synthetic peptide
YTSLIYSLLEKSQIQQEKNEQELLELDKWASLWNWF

SEQ ID NO: 18-21 - skipped (4 entries)

SEQ ID NO: 22 - protein, 33 aa, Artificial Sequence
  Description of Artificial Sequence: Synthetic peptide
YTSVITIELSNIKENKCNGAKVKLIKQELDKYK

SEQ ID NO: 23 - protein, 33 aa, Artificial Sequence
  Description of Artificial Sequence: Synthetic peptide
TSVITIELSNIKENKCNGAKVKLIKQELDKYKN

SEQ ID NO: 24 - protein, 33 aa, Artificial Sequence
  Description of Artificial Sequence: Synthetic peptide
VITIELSNIKENKCNGAKVKLIKQELDKYKNAV

SEQ ID NO: 25 - protein, 33 aa, Artificial Sequence
  Description of Artificial Sequence: Synthetic peptide
IALLSTNKAVVSLSNGVSVLTSKVLDLKNYIDK

SEQ ID NO: 26 - protein, 35 aa, Artificial Sequence
  Description of Artificial Sequence: Synthetic peptide
VEAKQARSDIEKLKEAIRDTNKAVQSVQSSIGNLI

SEQ ID NO: 27 - protein, 35 aa, Artificial Sequence
  Description of Artificial Sequence: Synthetic peptide
RSDIEKLKEAIRDTNKAVQSVQSSIGNLIVAIKSV

SEQ ID NO: 28 - protein, 35 aa, Artificial Sequence
  Description of Artificial Sequence: Synthetic peptide
NSVALDPIDISIELNKAKSDLEESKEWIRRSNQKL

SEQ ID NO: 29 - protein, 35 aa, Artificial Sequence
  Description of Artificial Sequence: Synthetic peptide
ALDPIDISIELNKAKSDLEESKEWIRRSNQKLDSI

SEQ ID NO: 30 - protein, 35 aa, Artificial Sequence
  Description of Artificial Sequence: Synthetic peptide
LDPIDISIELNKAKSDLEESKEWIRRSNQKLDSIG

SEQ ID NO: 31 - protein, 35 aa, Artificial Sequence
  Description of Artificial Sequence: Synthetic peptide
DPIDISIELNKAKSDLEESKEWIRRSNQKLDSIGN

SEQ ID NO: 32 - protein, 35 aa, Artificial Sequence
  Description of Artificial Sequence: Synthetic peptide
PIDISIELNKAKSDLEESKEWIRRSNQKLDSIGNW

SEQ ID NO: 33 - protein, 35 aa, Artificial Sequence
  Description of Artificial Sequence: Synthetic peptide
IDISIELNKAKSDLEESKEWIRRSNQKLDSIGNWH

SEQ ID NO: 34 - protein, 34 aa, Artificial Sequence
  Description of Artificial Sequence: Synthetic peptide
HRIDLGPPISLERLDVGTNLGNAIAKLEAKELLE

SEQ ID NO: 35 - protein, 34 aa, Artificial Sequence
  Description of Artificial Sequence: Synthetic peptide
IDLGPPISLERLDVGTNLGNAIAKLEAKELLESS

SEQ ID NO: 36 - protein, 34 aa, Artificial Sequence
  Description of Artificial Sequence: Synthetic peptide
LGPPISLERLDVGTNLGNAIAKLEAKELLESSDQ

SEQ ID NO: 37 - protein, 34 aa, Artificial Sequence
  Description of Artificial Sequence: Synthetic peptide
PISLERLDVGTNLGNAIAKLEAKELLESSDQILR

SEQ ID NO: 38 - protein, 35 aa, Artificial Sequence
  Description of Artificial Sequence: Synthetic peptide
WQEWERKVDFLEENITALLEEAQIQQEKNMYELQK

SEQ ID NO: 39 - protein, 35 aa, Artificial Sequence
  Description of Artificial Sequence: Synthetic peptide
QEWERKVDFLEENITALLEEAQIQQEKNMYELQKL

SEQ ID NO: 40 - protein, 35 aa, Artificial Sequence
  Description of Artificial Sequence: Synthetic peptide
EWERKVDFLEENITALLEEAQIQQEKNMYELQKLN

SEQ ID NO: 41 - protein, 35 aa, Artificial Sequence
  Description of Artificial Sequence: Synthetic peptide
WERKVDFLEENITALLEEAQIQQEKNMYELQKLNS

SEQ ID NO: 42 - protein, 35 aa, Artificial Sequence
  Description of Artificial Sequence: Synthetic peptide
ERKVDFLEENITALLEEAQIQQEKNMYELQKLNSW

SEQ ID NO: 43 - protein, 35 aa, Artificial Sequence
  Description of Artificial Sequence: Synthetic peptide
RKVDFLEENITALLEEAQIQQEKNMYELQKLNSWD

SEQ ID NO: 44 - protein, 35 aa, Artificial Sequence
  Description of Artificial Sequence: Synthetic peptide
KVDFLEENITALLEEAQIQQEKNMYELQKLNSWDV

SEQ ID NO: 45 - protein, 35 aa, Artificial Sequence
  Description of Artificial Sequence: Synthetic peptide
VDFLEENITALLEEAQIQQEKNMYELQKLNSWDVF

SEQ ID NO: 46 - protein, 35 aa, Artificial Sequence
  Description of Artificial Sequence: Synthetic peptide
DFLEENITALLEEAQIQQEKNMYELQKLNSWDVFG

SEQ ID NO: 47 - protein, 35 aa, Artificial Sequence
  Description of Artificial Sequence: Synthetic peptide
FLEENITALLEEAQIQQEKNMYELQKLNSWDVFGN

SEQ ID NO: 48 - protein, 8 aa, Artificial Sequence
  Description of Artificial Sequence: Synthetic peptide
WASLWNWF

SEQ ID NO: 49 - protein, 34 aa, Artificial Sequence
  Description of Artificial Sequence: Synthetic peptide
WMEWDREINNYTKLIHSLIEESQNQQEKNEQELL

SEQ ID NO: 50 - protein, 34 aa, Artificial Sequence
  Description of Artificial Sequence: Synthetic peptide
WMEWDREINKYTSLIHSLIEESQNQQEKNEQELL

SEQ ID NO: 51 - protein, 34 aa, Artificial Sequence
  Description of Artificial Sequence: Synthetic peptide
WMEWDREIKNYTSLIHSLIEESQNQQEKNEQELL

(19) United States
(12) Patent Application Publication
(10) Pub. No.: US 2009/0088378 A1
(43) Pub. Date: Apr. 2, 2009

(54) LONG LASTING INHIBITORS OF VIRAL INFECTION

(76) Inventors: Omar Quraishi, Westmount (CA); Cheryl A. Stoddart, Pacifica, CA (US); Paul L. Black, Bethesda, MD (US)

Correspondence Address: LOWRIE, LANDO & ANASTASI, LLP, ONE MAIN STREET, SUITE 1100, CAMBRIDGE, MA, 02142, US

(21) Appl. No.: 12/122,643

(22) Filed: May 16, 2008

Related U.S. Application Data
(63) Continuation-in-part of application No. 11/964,313, filed on Dec. 26, 2007.
(60) Provisional application No. 60/938,403, filed on May 16, 2007.
(60) Provisional application No. 60/884,865, filed on Jan. 12, 2007.

(51) Int. Cl. A61K 38/16; A61K 38/38; A61P 31/12

(52) U.S. Cl. 514/12

(57) ABSTRACT

This invention relates to C34 peptide derivatives that are inhibitors of viral infection and/or exhibit antifusogenic properties. In particular, this invention relates to C34 derivatives having inhibiting activity against human immunodeficiency virus (HIV), respiratory synctial virus (RSV), human parainfluenza virus (HPV), measles virus (MeV), and simian immunodeficiency virus (SIV) with long duration of action for the treatment of the respective viral infections.

### CROSS REFERENCE TO RELATED APPLICATIONS

This application claims priority to U.S. Ser. No. 60/938,403 which was filed on May 16, 2007. The contents of the aforementioned application are hereby incorporated by reference in their entirety.

### GOVERNMENT SUPPORT

The work described herein was carried out, at least in part, using funds from the United States government under contract number N01-AI-05418 and N01-AI-7002, from the National Institute of Allergy and Infectious Diseases/National Institutes of Health (NIAID/N1H). The government may therefore have certain rights in the invention.

### FIELD OF INVENTION

This invention relates to long lasting inhibitors of viral infection and/or exhibit antifusogenic properties or inhibit viral entry. In particular, this invention relates to long lasting inhibitors having inhibiting activity against human immunodeficiency virus (HIV), respiratory syncytial virus (RSV), human parainfluenza virus (HPV), measles virus (MeV), and simian immunodeficiency virus (SIV) with long duration of action for the treatment of the respective viral infections.

### BACKGROUND OF THE INVENTION

Entry of human immunodeficiency virus type 1 (HIV-1) into uninfected cells encompasses three main steps: (i) the binding of gp120 to the CD4 receptor, (ii) the subsequent binding to coreceptor CXCR4 or CCR5, and (iii) a series of conformational changes of the ectodomain of the HIV-1 transmembrane glycoprotein gp41 that are important to trigger membrane fusion that ultimately permits the infection process. Viruses such as respiratory syncytial virus (RSV), human parainfluenza virus type 3 (HPIV-3), measles virus and simian immunodeficiency virus (SIV) show a high degree of structural and functional similarity with HIV, including a gp41-like protein.

Several small molecule drug candidates, including those that inhibit binding to CD4 or to the CCR5 co-receptor, are either in human clinical trials or are close to market approval (Meanwell N A, Kadow J F (2003) Curr Opinion Drug Disc & Develop 6: 451-461; Olson W C, Maddon P J (2003) Curr Drug Targets-Infectious Disord 3: 283-294). Several synthetic peptides are known that inhibit or otherwise disrupt membrane fusion-associated events, including, for example, inhibiting retroviral transmission to uninfected cells. For example, the synthetic peptides C34, T1249, DP-107 and T-20 (DP-178), derived from separate domains within gp41, are potent inhibitors of HIV-1 infection and HIV induced cell-cell fusion.

T-20 (DP-178, enfuvirtide, Fuzeong, Trimeris/Roche Applied Sciences) is a synthetic peptide based on the CHR sequence of HIV-1 gp41, and is believed to target the conformational rearrangements of gp41. It had been widely believed that T-20 inhibition was due to its ability to bind to the hydrophobic grooves of the NHR region of gp41 resulting in the inhibition of six-helix bundle formation (Kliger Y, Shai Y (2000) J Mol Biol 295: 163-168). Contrary to this view, recent studies have suggested that T-20 is capable of targeting multiple sites in gp41 and gp120 (Liu S et al. (2005) J Biol Chem 280:11259-11273). For example, T-20 binds and oligomerizes at the surface of membranes, thereby inhibiting recruitment and oligomerization of gp41 at the plasma membrane of infected cells (Muñioz-Barroso I et al. (1998) J Cell Biol 140: 315-23; Kliger Y et al. (2001) J Biol Chem 276:1391-1397). Furthermore, it has also been shown that the ectodomain of gp41 within a region immediately adjacent to the membrane-spanning domain having the peptide sequence, ⁶⁶⁶WASLWNWF⁶⁷³, constitutes a higher affinity site for T-20 than the NHR of gp41 (Muñoz-Barroso I et al. (1998) supra 140: 315-23; Kliger Y et al. (2001) supra).

Another C-peptide, C34, composed of a peptide sequence which overlaps with T-20 but contains the gp41 coiled-coil cavity binding residues, ⁶²⁸WMEW⁶³¹, is known to compete with the CHR of gp41 for the hydrophobic grooves of the NHR region (Liu S et al. (2005) J Biol Chem 280:11259-11273).

While many of the antiviral or antifusogenic peptides described in the art exhibit potent antiviral and/or antifusogenic activity, these peptides suffer from short plasma half-lifes in vivo. There is therefore a need for a method of prolonging the half-life of existing antiviral and/or antifusogenic peptides, thus providing for longer acting antiviral and/or antifusogenic peptides in vivo.

### SUMMARY OF THE INVENTION

The present invention is directed to, at least in part, modified antiviral and/or antifusogenic peptides, and conjugates thereof, having a longer acting antiviral and/or antifusogenic effect in vivo, compared to the peptides prior to modification. The modified peptides, can include chemically reactive moieties such that the modified peptides can react with available functionalities on blood components, e.g., albumin, thus increasing the stability in vivo of the modified peptides. These modified peptides, and conjugates thereof, thereby minimize, e.g., the need for more frequent, or even continual, administration of the peptides. The modified peptides, and conjugates thereof, of the present invention can be used, e.g., prophylactically against and/or therapeutically for ameliorating infection of a number of viruses, including human immunodeficiency virus (HIV), human respiratory syncytial virus (RSV), human parainfluenza virus (HPV), measles virus (MeV) and simian immunodeficiency virus (SIV). Modification of other peptides involved in viral transfection (e.g., Hepatitis, Epstein Barr and other related viruses) is also within the scope of the invention.

In accordance with the present invention, there is now provided modified peptides such as viral inhibitor derivatives (e.g., C34 peptide derivatives), and conjugates thereof, having an extended in vivo half-life when compared with the corresponding unmodified viral inhibitor (e.g., C34 peptide sequence).

The present invention includes compounds having a viral inhibitor (e.g an amino acid sequence of C34), a linker, along with a reactive group capable of reacting with thiol groups on a blood component (or blood protein), either in vivo or ex vivo, to form a stable covalent bond (e.g., the viral inhibitor or modified antifusogenic peptide is covalently coupled to a blood protein). Preferred blood components comprise proteins such as immunoglobulins, including IgG and IgM, serum albumin, ferritin, steroid binding proteins, transferrin, thyroxin binding protein, α-2-macroglobulin etc., serum albumin and IgG being more preferred, and serum albumin, e.g., human serum albumin being the most preferred. Albumin, and other blood proteins, may also be derived from a recombinant or genomic source, such as yeast, bacteria (e.g., E. coli), mammalian cells (e.g., Chinese hamster ovary (CHO) cells), transgenic plant, transgenic animal, etc. In certain embodiments, the modified antifusogenic peptide may be covalently coupled to the Cys34 residue of albumin.

In one embodiment, the modified antiviral and/or antifusogenic peptide includes at least a portion of a gp41 coiled-coil cavity binding residues. For example, the peptide can include residues 628WMEW631 (SEQ ID NO:1), or up to one amino acid substitution (e.g., conservative or non-conservative substitution), deletions, or insertions thereto. In other embodiments, the antiviral and/or antifusogenic peptide includes the full or partial native amino acid sequence of C34 from amino acids ⁶²⁸WMEWDREINNYTSLIHSLIEESQNQQEKNEQELL⁶⁶¹ (corresponding to amino acids C1 to C34) (SEQ ID NO:2), or up to five, four, three, two or one amino acid substitutions (e.g., conservative or non-conservative substitution), insertions or deletions thereto.

In other embodiments, the modified antiviral and/or antifusogenic peptide includes the amino acid sequence of DP107 and DP178 peptides and analogs thereof, including peptides comprised of amino acid sequences from other (non-HIV) viruses that correspond to the gp41 region of HIV from which DP107 and DP178 are derived and that exhibit antiviral and/or antifusogenic activity. More particularly, these peptides can exhibit antiviral activity against, among others, human respiratory syncytial virus (RSV), human parainfluenza virus (HPV), measles virus (MeV) and simian immunodeficiency virus (SIV). The invention also relates to modified peptides of SEQ ID NO: 1 to SEQ ID NO:86 of US 05/0070475, the entire contents of which are incorporated by reference herein in their entirety.

In embodiments, the modified antiviral and/or antifusogenic peptides, of the invention further include one or more chemically reactive moieties or groups such that the modified peptides can react with available functionalities on blood components to form stable covalent bonds, thereby producing conjugated peptide forms. In one embodiment of the invention, the modified peptide comprises one or more reactive groups which react with one or more amino groups, hydroxyl groups, or thiol groups on one or more blood components (e.g., albumin) to form stable covalent bonds. In another embodiment, the reactive group can be a maleimide-containing group (e.g., MPA (maleimido propionic acid) or GMBA (gamma-maleimide-butyralamide)) which is reactive with a thiol group on a blood protein, including a mobile blood protein such as albumin. The reactive modification or group can further include one or more linkers. In embodiments, the linker is chosen from one or more of: (2-amino)ethoxy acetic acid (AEA), [2-(2-amino)ethoxy)]ethoxy acetic acid (AEEA), ethylenediamine (EDA); one or more alkyl chains (C1-C10) such as 8-aminooctanoic acid (AOA), 8-aminopropanoic acid (APA), or 4-aminobenzoic acid (APhA). The reactive group, with or without linker, can be added to the N- or C-terminal of the antiviral and/or antifusogenic modified peptide, typically, the C-terminal of the antiviral and/or antifusogenic modified peptide. In embodiments, the reactive group is attached to an internal residue of the modified peptide (e.g., attached to an epsilon NH₂ group of an internal lysine residue; a hydroxyl group of an internal serine residue (e.g., Serine 13 of C34)). Non-limiting examples of C34 modified peptides are disclosed in WO 02/096935, the entire contents of which are incorporated by reference herein in their entirety.

Non-limiting examples of modified antiviral and/or antifusogenic modified peptides of C34 of the present invention include the following sequences, which are capable of reacting with thiol groups on a blood component either in vivo or ex vivo, to form a stable covalent bond:

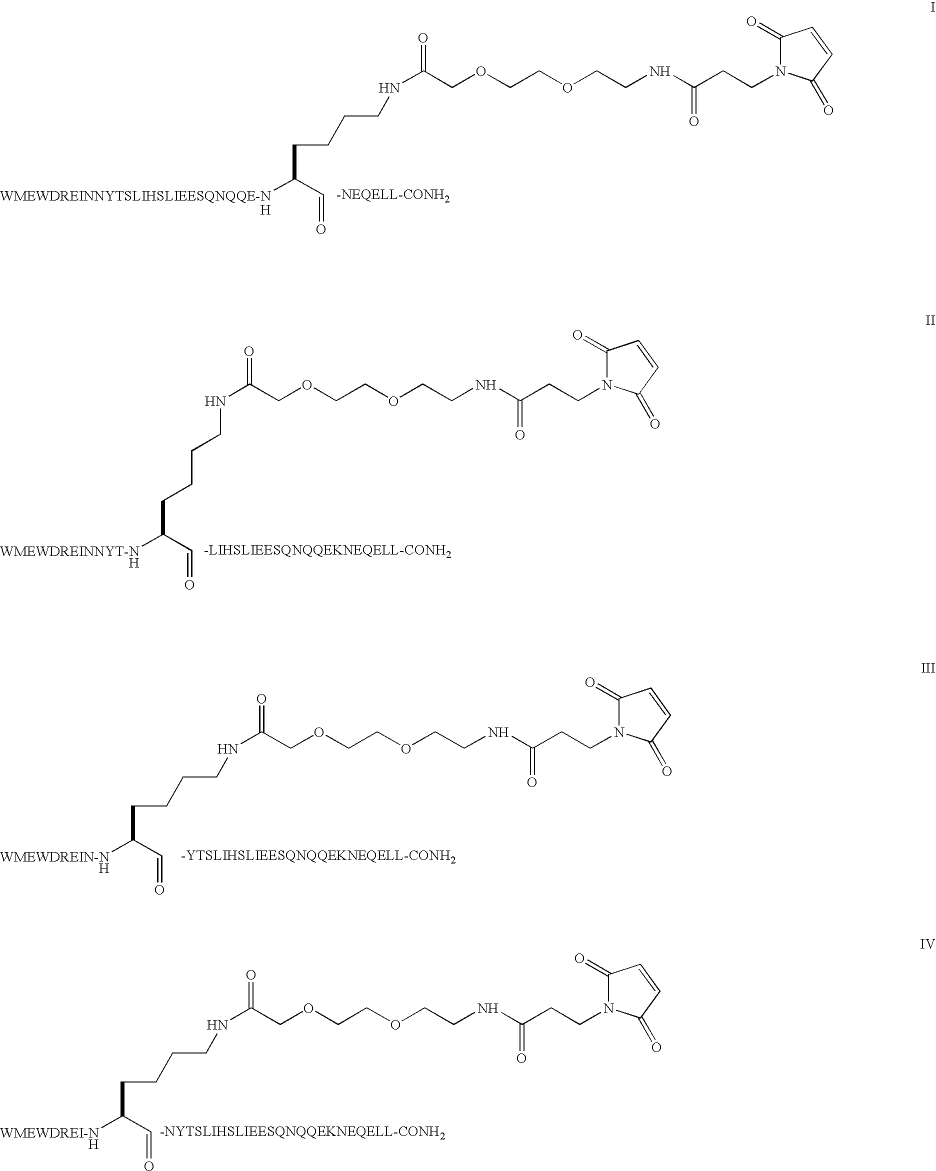

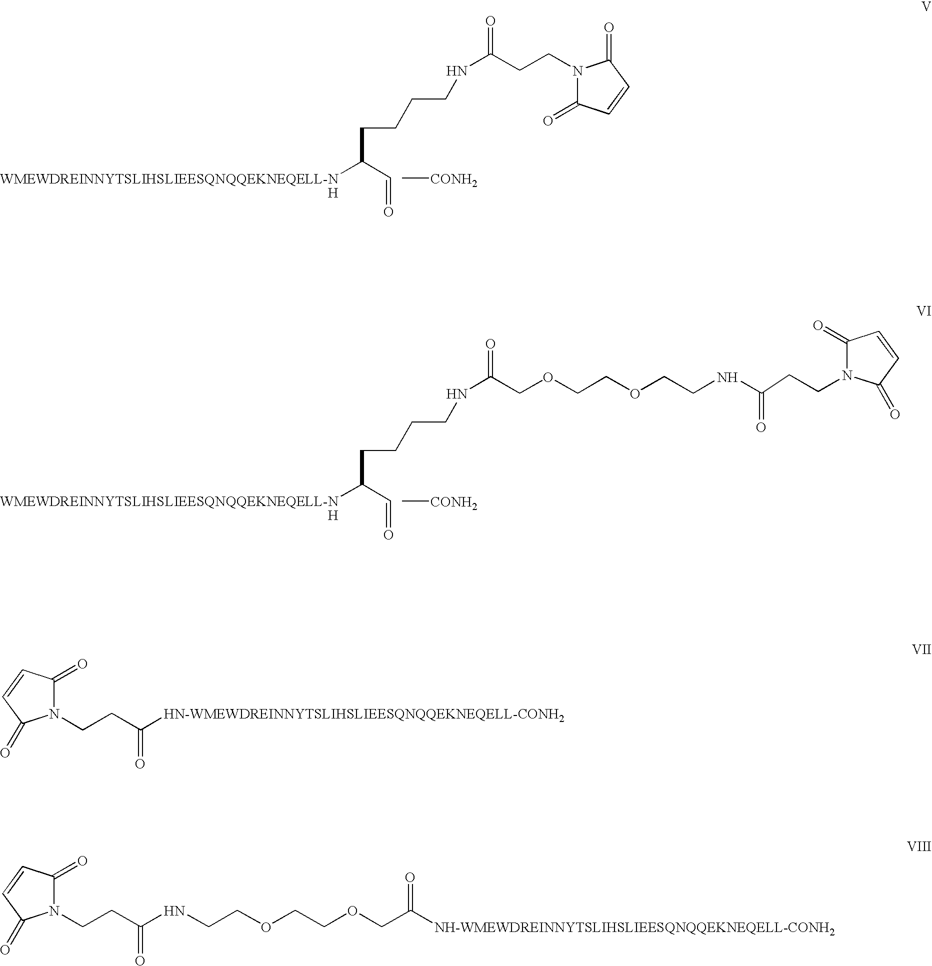

In yet another aspect, the invention features conjugates of the modified antiviral and/or antifusogenic peptides described herein having one or more chemically reactive modifications coupled to available functionalities on one or more blood components. In one embodiment of the invention, the modified peptides comprise a reactive group which is coupled to amino groups, hydroxyl groups, or thiol groups on blood components to form stable covalent bonds. The maleimide group can be directly coupled to the modified peptide or can be coupled indirectly, e.g., via a linker. In another embodiment of the invention, the reactive group can be a maleimide which is reactive with a thiol group on a blood protein, including a mobile blood protein such as albumin.

The modified antiviral and/or antifusogenic peptide can include a reactive moiety, e.g., a maleimide-containing group, that has the ability to covalently bond one or more blood components, e.g., serum albumin, so as to form a conjugate. The conjugation step can occur in vivo, e.g., after administration of the modified peptide to a subject. Alternatively, the conjugation step can occur ex vivo or in vitro, e.g., by contacting the modified peptide containing the reactive group with a blood components, e.g., albumin. The preparation and uses of conjugates of C34, DP107, DP178 and the like are disclosed in WO 02/096935 and US 05/0070475, incorporated by reference herein in their entirety. The conjugates formed in vivo, or ex vivo are useful in inhibiting the viral and/or fusogenic activity of viruses, such as HIV, RSV, HPV, MeV or SIV in a subject, e.g., a human subject.

In another aspect, the invention features, compositions, e.g., pharmaceutical compositions, or dosage formulations, that include one or more modified antiviral and/or antifusogenic peptides as described herein. In embodiments, the compostions are suitable for injection (e.g., subcutaneous or intravascular injection, pulmonary inhalation, intraperitoneal, or intramuscularly). In another aspect of the invention, there is provided a pharmaceutical composition comprising the viral inhibitor derivatives in combination with a pharmaceutically acceptable carrier. Such composition is useful for inhibiting the activity of HIV, RSV, HPV, MeV or SIV. The compositions, e.g., pharmaceutical compositions or dosage formulations may be suitable for prophylactic use or therapeutic use, and may also be suitable for administering as an initial dose for treating a viral infection.

In another aspect, the invention features methods and compositions for use in the prevention and/or treatment of viral infection comprising a modified antiviral and/or antifusogenic peptide or conjugate thereof, as described herein. The method includes administering to a subject (e.g., a human subject) in need of treatment an effective amount, e.g., a prophylactic or therapeutic amount, of a modified antiviral and/or antifusogenic peptide or conjugate thereof, as described herein. Exemplary viral infections that can be treated or prevented include AIDS, human respiratory syncytial virus (RSV), human parainfluenza virus (HPV), measles virus (MeV) and simian immunodeficiency virus (SIV).

Methods and compositions for inhibiting one or more activities of HIV, RSV, HPV, MeV or SIV in a subject, e.g., a human subject, are disclosed. The method includes administering to a subject in need of treatment an effective amount, e.g., a prophylactic or therapeutic amount, of a modified antiviral and/or antifusogenic peptide or conjugate thereof, as described herein. The subject may be a subject that has or is at the risk of having a virus, e.g., HIV, RSV, HPV, MeV or SIV. In a further embodiment of the present invention, there is provided a method for inhibiting the activity of HIV, RSV, HPV, MeV or SIV. The method comprises administering to a subject, preferably a mammal, an effective amount of the viral inhibitor derivatives alone or in combination with a pharmaceutical carrier.

In the case of prophylactic use (e.g., to prevent, reduce, or delay onset or recurrence of one or more symptoms of the disorder or condition), the subject may or may not have one or more symptoms of the infection. For example, the antifusogenic and/or antiviral peptide may be administered prior to any detectable manifestation of the symptoms, or after at least some, but not all of the systems are detected. In the case of therapeutic use, the treatment may improve, cure, maintain, or decrease duration of, the disorder or condition in the subject. In therapeutic uses, the subject may have a partial or full manifestation of the symptoms. In a typical case, treatment improves the disorder or condition of the subject to an extent detectable by a physician, or prevents worsening of the disorder or condition.

In some embodiments, a method of treating or preventing a virus selected from the group consisting of human immunodeficiency virus (HIV) infection, respiratory syncytial virus (RSV), human parainfluenza virus type 3 (HPIV-3), measles virus and simian immunodeficiency virus (SIV) in a subject is provided comprising administering a modified antifusogenic peptide to a subject having or at risk of the virus as an initial dose(s), thereby treating or preventing the infection.

In one embodiment, the antiviral and/or antifusogenic peptide is administered as one or more initial dose(s) prior to infection or to the onset or recurrence of one or more symptoms associated with the infection. As used herein “an initial dose” refers to an amount and/or frequency of administration of a modified antiviral or antifusogenic peptide, or conjugate thereof, that when administered as a single dose, or as repeated doses, reduces the severity of, ameliorates, prevents, or delays the occurrence or recurrence of infection. The antiviral and/or antifusogenic peptide can be administered as an initial dose prior to infection or to the onset or recurrence of one or more symptoms associated with the infection, but before a full manifestation of the symptoms associated with the infection. In certain embodiments, for example, the initial single dose may be administered as a single treatment interval, e.g., as a single initial dose, or as multiple repeated doses. In certain embodiments, the antiviral and/or antifusogenic peptide is administered to the subject prior to exposure to an agent that triggers or exacerbates an associated disorder or condition e.g., a virus or fusogenic agent, or an infection. Each dose can be administered by pulmonary inhalation, intraperitoneal, or by injection, e.g., intramuscularly, or subcutaneously in an amount of about 1 mg/kg to about 400 mg/kg (e.g., at least about 1 mg/kg, 3 mg/kg, 10 mg/kg, 30 mg/kg, 50 mg/kg, 60 mg/kg, 75 mg/kg, 100 mg/kg, 150 mg/kg, 200 mg/kg, 250 mg/kg, or 300 mg/kg). Dosages for the initial dose can be in an amount of about 50 mg/kg to about 400 mg/kg (e.g., at least about 50 mg/kg, 60 mg/kg, 75 mg/kg, 100 mg/kg, 150 mg/kg, 200 mg/kg, 250 mg/kg, or 300 mg/kg).

In some embodiments one or more subsequent doses of the antiviral and/or antifusogenic peptide can be administered after administering the single initial dose. Dosages for the subsequent dose can be in an amount of 1 mg/kg to about 400 mg/kg (e.g., at least about 1 mg/kg, 3 mg/kg, 10 mg/kg, 30 mg/kg, 50 mg/kg, 60 mg/kg, 75 mg/kg, 100 mg/kg, 150 mg/kg, 200 mg/kg, 250 mg/kg, or 300 mg/kg). Dosages for the initial dose can be in an amount of about 50 mg/kg to about 400 mg/kg (e.g., at least about 50 mg/kg, 60 mg/kg, 75 mg/kg, 100 mg/kg, 150 mg/kg, 200 mg/kg, 250 mg/kg, or 300 mg/kg).

In some embodiments, the initial dose(s) or subsequent dose(s) of antiviral and/or antifusogenic peptide is administered prior to, during, or shortly after exposure to the agent that triggers and/or exacerbates an infection. For example, the antiviral and/or antifusogenic peptide can be administered 1, 5, 10, 20, or 24 hours, 2, 3, 4, 5, 10, 15, 20, or 30 days; or 4, 5, 6, 7 or 8 weeks, or more before or after exposure to the triggering or exacerbating agent, virus or fusogenic agent. Typically, the antiviral and/or antifusogenic peptide can be administered 24 hours, to 3 days before or after exposure to the triggering or exacerbating agent.

In some embodiments, the administration of the dose may be repeated by administering a subsequent dose anywhere between every 6 hours, 12 hours, 24 hours, 3 days, 4 days, or 7 days. The subsequent dose may be repeated at least once, or for an indefinite period of time. In those embodiments where administration occurs after exposure to the agent, the subject may not be experiencing symptoms or may be experiencing a partial manifestation of the symptoms. The administration of the dose may occur 3 days after exposure to the agent or infection. For example, the subject may have symptoms of an early stage of the infection.

In some embodiments the modified antifusogenic peptide may be administered at a time interval. For example, the subsequent does may be administered at time intervals selected from the group consisting of 1 hour, 2 hours, 4 hours, 8 hours, 12 hours, 24 hours, 4 days, 7 days, 14 days, 30 days, and 60 days.

In some embodiments, the method of treating or preventing a virus further comprises selecting a subject from a group of subjects prior to infection or to the onset or recurrence of one or more symptoms associated with the infection.

In a further aspect of the present invention, there is provided a conjugate comprising a viral inhibitor derivative covalently bonded to a blood component through a compound having a structure of Formulae I-V.

In a further aspect of the present invention, there is provided a method for extending the in vivo half-life of a viral inhibitor in a subject, the method comprising covalently bonding, e.g., through a compound of Formulae I-V to a blood component. In one embodiment, the viral inhibitor is covalently bound to a blood component through a compound provided below:

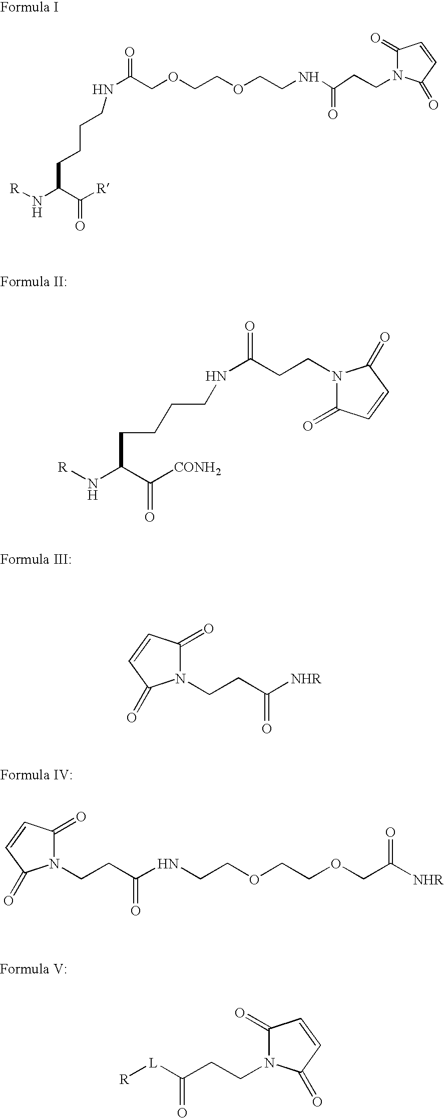

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The contents of all publications, pending patent applications, published patent applications (inclusive of WO 02/096935 and US 05/0070475), and published patents cited throughout this application are hereby incorporated by reference in their entirety.

Others features, objects and advantages of the invention will be apparent from the description and drawings, and from the claims.

### BRIEF DESCRIPTION OF THE DRAWINGS

FIG. 1 is a schematic diagram showing the structure of Compound VIII. The linker is amino ethoxy ethoxy acetic acid (AEEA) and the one-letter amino acid code derived from HIV-1 HXB2 is used.

FIG. 2 illustrates that Compound VIII is three times more potent than maleimido-Compound VIII in reducing viral RNA in SC-hu Thy/Liv mice. Mice were treated twice daily by subcutaneous injection beginning one day before inoculation of Thy/Liv implants with 1,000 TCID₅₀ HIV-1 NL4-3G. Columns represent means and open circles individual animals from the same cohort 21 days after innoculation. The limit of detection of HIV-1 RNA by the bDNA assay was 1.5 log₁0 copies per 106 implant cells. *P<0.05, **P<0.01 compared to untreated mice by the Mann-Whitney U test.

FIG. 3A illustrates Compound VIII and T-20 are equipotent against NL4-3G when administered twice daily. SCID-hu Thy/Liv mice were treated by subcutaneous injection beginning one day before inoculation. Antiviral efficacy was assessed by determining HIV-1 RNA, p24, percentage of Gag-p24⁺ thymocytes, and MHC-1 expression on DP thymocytes.

FIG. 3B illustrates Compound VIII and T-20 are equipotent against NL4-3G when administered twice daily. SCID-hu Thy/Liv mice were treated by subcutaneous injection beginning one day before inoculation. Thymocyte protection was assessed by total implant cellularity, thymocyte viability, percentage of DP thymocytes, and CD4/CD8 ratio for treated versus untreated mice. Columns represent means and open circles individual animals from the same cohort 21 days after virus (or mock) inoculation. The limit of detection of HIV-1 RNA by the bDNA assay was 1.5 log₁₀ copies per 10⁶ implant cells. *P<0.05, **P<0.01 compared to untreated mice by the Mann-Whitney U test.

FIG. 4A illustrates that Compound VIII is >10 times more potent than T-20 against T-20-resistant NL4-3D. SCID-hu Thy/Liv mice were treated twice daily by subcutaneous injection beginning 24 h before inoculation. Viral RNA means (columns) and individual animals (open circles) from one cohort 21 days after inoculation. **P<0.01 by the Mann-Whitney U test.

FIG. 4B illustrates that Compound VIII is >10 times more potent than T-20 against T-20-resistant NL4-3D. SCID-hu Thy/Liv mice were treated twice daily by subcutaneous injection beginning 24 h before inoculation. Reduction in viral RNA by treatment with Compound VIII or T-20 in NL4-3G- and NL4-3D-infected mice. Each point represents the log₁₀ difference in means between treated and untreated groups (5-7 mice per group) in seven separate experiments with a total of 202 mice.

FIG. 5. Compound VIII has more sustained activity than T-20 against NL4-3G when administered every fourth or seventh day. SCID-hu Thy/Liv mice were treated by subcutaneous injection of 30 mg/kg beginning 1 day before (−1) or 3 days after (+3) inoculation and continued every fourth day (Q4D) or every seventh day (Q7D) until implant collection. Antiviral efficacy was assessed by determining HIV-1 RNA and p24, and thymocyte protection was assessed by percentage of DP thymocytes. Columns represent means and open circles individual animals from the same cohort 21 days after virus (or mock) inoculation. *P<0.05, **P<0.01 by the Mann-Whitney U test.

FIG. 6. Compound VIII has more sustained activity than T-20 against NL4-3G with a single preexposure dose. SCID-hu Thy/Liv mice were treated with one subcutaneous injection of 200 mg/kg 1 day before inoculation. Antiviral efficacy was assessed by determining HIV-1 RNA and p24, and thymocyte protection was assessed by percentage of DP thymocytes. Columns represent means and open circles individual animals from the same cohort 21 days after virus (or mock) inoculation. *P<0.05, **P<0.01 by the Mann-Whitney U test.

### DETAILED DESCRIPTION OF THE INVENTION

The present invention meets these and other needs and is directed to viral inhibitor derivatives having antiviral activity and/or antifusogenic activity.

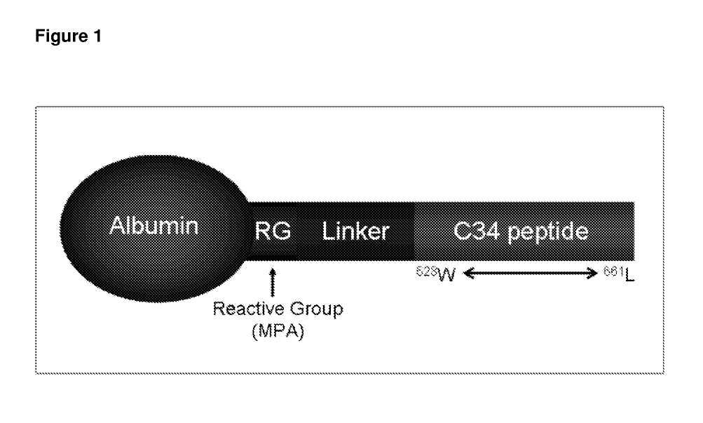

In the present invention, C34 peptide may be derived from the C-terminal helical region (CHR) of gp41 (Chan D C, et al. (1997) Cell 89: 263-273; Chan D C, et al. (1998) Proc Natl Acad Sci USA 95: 15613-15617) was engineered into preformed albumin conjugates whereby specific covalent linkage to albumin was carried out through either the N-terminus or the C-terminus of the fusion inhibitor. Similarly, preformed albumin conjugates composed of maleimido-T-20 analogs were also generated. Each drug construct represented a 1:1 complex through specific and stable covalent attachment of the peptide to cysteine-34 of albumin, and each construct was assessed for its antiviral activity in vitro following infection in a peripheral blood mononuclear cell (PBMC)-based assay with the HIV-1 strain IIIB (Popovic M E, et al. (1984) Lancet ii: 1472-1473; Popovic M, et al. (1984) Science 224:497-500; Ratner L et al. (1985) Nature 313:277-283; Buckheit R W, Swanstrom R (1991) AIDS Res Hum Retrovir 7:295-302) Furthermore, using the SCID-hu Thy/Liv mouse model of HIV-1 infection (Stoddart C A et al. (2007) PLoS ONE 2:e655), we evaluate the antiviral activity of one C34-HSA conjugate, Compound VIII (FIG. 1), and found that while T-20 lost activity with infrequent dosing, the antiviral potency of Compound VIII was sustained.

Certain terms are defined herein as follows:

Antiviral peptides: As used herein, “antiviral peptides” shall refer to peptides that inhibit viral infection of cells, by, for example, inhibiting cell-cell fusion or free virus infection. The route of infection may involve membrane fusion, as occurs in the case of enveloped viruses, or some other fusion event involving viral and cellular structures. Peptides that inhibit viral infection by a particular virus may be referenced with respect to that particular virus, e.g., anti-HIV peptide, anti-RSV peptide, among others.

Antifusogenic peptides: “Antifusogenic peptides” are peptides demonstrating an ability to inhibit or reduce the level of membrane fusion events between two or more entities, e.g., virus-cell or cell-cell, relative to the level of membrane fusion that occurs in the absence of the peptide.

HIV and anti-HIV peptides: The human immunodeficiency virus (HIV), which is responsible for acquired immune deficiency syndrome (AIDS), is a member of the lentivirus family of retroviruses. There are two prevalent types of HIV, HIV-1 and HIV-2, with various strain of each having been identified. HIV targets CD-4+ cells, and viral entry depends on binding of the HIV protein gp41 to CD-4+ cell surface receptors. Anti-HIV peptides refer to peptides that exhibit antiviral activity against HIV, including inhibiting CD-4+ cell infection by free virus and/or inhibiting HIV-induced syncytia formation between infected and uninfected CD-4+ cells.

SIV and anti-SIV peptides: Simian immunodeficiency viruses (SIV) are lentiviruses that cause acquired immunodeficiency syndrome (AIDS)-like illnesses in susceptible monkeys. Anti-SIV peptides are peptides that exhibit antiviral activity against SIV, including inhibiting of infection of cells by the SIV virus and inhibiting syncytia formation between infected and uninfected cells.

RSV and anti-RSV peptides: Respiratory syncytial virus (RSV) is a respiratory pathogen, especially dangerous in infants and small children where it can cause bronchiolitis (inflammation of the small air passages) and pneumonia. RSVs are negative sense, single stranded RNA viruses and are members of the Paramyxoviridae family of viruses. The route of infection of RSV is typically through the mucous membranes by the respiratory tract, e.g., nose, throat, windpipe and bronchi and bronchioles. Anti-RSV peptides are peptides that exhibit antiviral activity against RSV, including inhibiting mucous membrane cell infection by free RSV virus and syncytia formation between infection and uninfected cells.

HPV and anti-HPV peptides: Human parainfluenza virus (HPIV or HPV), like RSV, is another leading cause of respiratory tract disease, and like RSVs, are negative sense, single stranded RNA viruses that are members of the Paramyxoviridae family of viruses. There are four recognized serotypes of HPIV-HPIV-1, HPIV-2, HPIV-3 and HPIV-4. HPIV-1 is the leading cause of croup in children, and both HPIV-1 and HPIV-2 cause upper and lower respiratory tract illnesses. HPIV-3 is more often associated with bronchiolitis and pneumonia. Anti-HPV peptides are peptides that exhibit antiviral activity against HPV, including inhibiting infection by free HPV virus and syncytia formation between infected and uninfected cells.

MeV and anti-Mev peptides: Measles virus (VM or MeV) is an enveloped negative, single-stranded RNA virus belonging to the Paramyxoviridae family of viruses. Like RSV and HPV, MeV causes respiratory disease, and also produces an immuno-suppression responsible for additional, opportunistic infections. In some cases, MeV can establish infection of the brain leading to severe neurological complications. Anti-MeV peptides are peptides that exhibit antiviral activity against MeV, including inhibiting infection by free MeV virus and syncytia formation between infected and uninfected cells.

C34 and C34 analogs: The term “C34” refers to a portion of a gp41 coiled-coil cavity binding residues. For example, the peptide can include residues ⁶²⁸WMEW⁶³¹ of gp41 (SEQ ID NO:1), or ⁶²⁸WMEWDREINNYTSLIHSLIEESQNQQEKNEQELL⁶⁶¹ of gp41 (SEQ ID NO:2).

Analogs of C34 can include truncations, deletions, insertions and/or amino acid substitutions (e.g., conservative or non-conservative substitution) thereof. Deletions may consist of the removal of one or more amino acid residues from the C34 peptide, and may involve the removal of a single contiguous portion of the peptide sequence or multiple portions. Insertions may comprise single amino acid residues or stretches of residues and may be made at the carboxy or amino terminal end of the C34 peptide or at a position internal to the peptide.

DP-178 and DP178 analogs: Unless otherwise indicated explicitly or by context, DP-178 means the 36 amino acid DP-178 peptide corresponding to amino acid residues 638-673 of the gp41 glycoprotein of HIV-1 isolate LAI (HIV_{LAI}) and having the sequence:

| | |
|---|---|
| YTSLIHSLIEESQNQQEKNEQELLELDKWASLWNWF | (SEQ ID NO:3) |

Analogs of DP178 can include truncations, deletions, insertions and/or amino acid substitutions (e.g., conservative or non-conservative substitution) thereof. Truncations of the peptide may comprise peptides of between 3-36 amino acids. Deletions may consist of the removal of one or more amino acid residues from the DP 178 peptide, and may involve the removal of a single contiguous portion of the peptide sequence or multiple portions. Insertions may comprise single amino acid residues or stretches of residues and may be made at the carboxy or amino terminal end of the DP178 peptide or at a position internal to the peptide.

DP178 peptide analogs are peptides whose amino acid sequences are comprised of the amino acid sequences of peptide regions of viruses other than HIV-1_{LAI} that correspond to the gp411 region from which DP178 was derived, as well as an truncations, deletions or insertions thereof. Such other viruses may include, but are not limited to, other HIV isolates such as HIV-2_{NIHZ}, respiratory syncytial virus (RSV), human parainfluenza virus (HPV), simian immunodeficiency virus (SIV), and measles virus (MeV). DP178 analogs also refer to those peptide sequences identified or recognized by the ALLMOTI5, 107 X 178 X 4 and PLZIP search motifs described in U.S. Pat. Nos. 6,013,263, 6,017,536 and 6,020,459 and incorporated herein, having structural and/or amino acid motif similarity to DP178. DP178 analogs further refer to peptides described as “DP178-like” as that term is defined in U.S. Pat. Nos. 6,013,263, 6,017,536 and 6,020,459.

DP-107 and DP107 analogs: Unless otherwise indicated explicitly or by context, DP-107 means the 38 amino acid DP-107 peptide corresponding to amino acid residues 558-595 of the gp41 protein of HIV-1 isolate LAI (HIV_{LAI}) and having the sequence:

(SEQ ID NO:4)

NNLLRAIEAQQHLLQLTVWQIKQLQARILAVERYLKDQ.

Analogs of DP107 can include truncations, deletions, insertions and/or amino acid substitutions (e.g., conservative or non-conservative substitution) thereof. Truncations of the peptide may comprise peptides of between 3-38 amino acids. Deletions may consist of the removal of one or more amino acid residues from the DP107 peptide, and may involve the removal of a single contiguous portion of the peptide sequence or multiple portions. Insertions may comprise single amino acid residues or stretches of residues and may be made at the carboxy or amino terminal end of the DP107 peptide or at a position internal to the peptide.

DP107 peptide analogs are peptides whose amino acid sequences are comprised of the amino acid sequences of peptide regions of viruses other than HIV-1_{LAI} that correspond to the gp41 region from which DP107 was derived, as well as truncations, deletions and/or insertions thereof. Such other viruses may include, but are not limited to, other HIV isolates such as HIV-2_{NIHZ}, respiratory syncytial virus (RSV), human parainfluenza virus (HPV), simian immunodeficiency virus (SIV), and measles virus (MeV). DP107 analogs also refer to those peptide sequences identified or recognized by the ALLMOTI5, 107 X 178 X 4 and PLZIP search motifs described in U.S. Pat. Nos. 6,013,263, 6,017,536 and 6,020,459 and incorporated herein, having structural and/or amino acid motif similarity to DP107. DP107 analogs further refer to peptides described as “DP107-like” as that term is defined in U.S. Pat. Nos. 6,013,263, 6,017,536 and 6,020,459.

Reactive Groups: Reactive groups are chemical groups capable of forming a covalent bond. Such reactive groups are coupled or bonded to a C34, DP-107, DP-178 or T-1249 peptide or analogs thereof or other antiviral or antifusogenic peptide of interest. Reactive groups will generally be stable in an aqueous environment and will usually be carboxy, phosphoryl, or convenient acyl group, either as an ester or a mixed anhydride, or an imidate, thereby capable of forming a covalent bond with functionalities such as an amino group, a hydroxy or a thiol at the target site on mobile blood components. For the most part, the esters will involve phenolic compounds, or be thiol esters, alkyl esters, phosphate esters, or the like.

Functionalities: Functionalities are groups on blood components to which reactive groups on modified antiviral peptides react to form covalent bonds. Functionalities include hydroxyl groups for bonding to ester reactive entities; thiol groups for bonding to maleimides, imidates and thioester groups; amino groups for bonding to carboxy, phosphoryl or acyl groups and carboxyl groups for bonding to amino groups.

Blood Components or Carrier Proteins: Blood components may be either fixed or mobile. Fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1.mu.g/ml. Mobile blood components include carrier proteins. Mobile blood components include serum albumin, transferrin, ferritin and immunoglobulins such as IgM and IgG. The half-life of mobile blood components is at least about 12 hours. Additional examples of blood components include ferritin, steroid binding proteins, transferrin, thyroxin binding protein, and α-2-macroglobulin. Typically, serum albumin and IgG being more preferred, and serum albumin, e.g., human serum albumin being the most preferred. Albumin may also be derived from a recombinant or genomic source, such as yeast, bacteria (e.g. E. coli), mammalian cells (e.g. Chinese hamster ovary (CHO) cells), transgenic plant, transgenic animal, Thus, the term “blood component” includes proteins that are biochemically purified from a subject, as well as proteins made recombinantly.

Protective Groups: Protective groups are chemical moieties utilized to protect peptide derivatives from reacting with themselves. Various protective groups are disclosed herein and in U.S. Pat. No. 5,493,007, which is hereby incorporated by reference. Such protective groups include acetyl, fluorenylmethyloxycarbonyl (Fmoc), t-butyloxycarbonyl (Boc), benzyloxycarbonyl (CBZ), and the like. The specific protected amino acids are depicted in Table 1.

TABLE 1
NATURAL AMINO ACIDS AND THEIR ABBREVIATIONS

| | 3-Letter | 1-Letter | |
|---|---|---|---|
| Name | Abbreviation | Abbreviation | Modified Amino Acids |
| Alanine | Ala | A | Fmoc-Ala-OH |
| Arginine | Arg | R | Fmoc-Arg(Pbf)-OH |
| Asparagine | Asn | N | Fmoc-Asn(Trt)-OH |
| Aspartic acid | Asp | D | Asp(tBu)-OH |
| Cysteine | Cys | C | Fmoc-Cys(Trt) |
| Glutamic acid | Glu | E | Fmoc-Glu(tBu)-OH |
| Glutamine | Gln | Q | Fmoc-Gln(Trt)-OH |
| Glycine | Gly | G | Fmoc-Gly-OH |
| Histidine | His | H | Fmoc-His(Trt)-OH |
| Isoleucine | Ile | I | Fmoc-Ile-OH |
| Leucine | Leu | L | Fmoc-Leu-OH |
| Lysine | Lys | Z | Boc-Lys(Aloc)-OH |
| Lysine | Lys | X | Fmoc-Lys(Aloc)-OH |
| Lysine | Lys | K | Fmoc-Lys(Mtt)-OH |
| Methionine | Met | M | Fmoc-Met-OH |
| Phenylalanine | Phe | F | Fmoc-Phe-OH |
| Proline | Pro | P | Fmoc-Pro-OH |
| Serine | Ser | S | Fmoc-Ser(tBu)-OH |
| Threonine | Thr | T | Fmoc-Thr(tBu)-OH |
| Tryptophan | Trp | W | Fmoc-Trp(Boc)-OH |
| Tyrosine | Tyr | Y | Boc-Tyr(tBu)-OH |
| Valine | Val | V | Fmoc-Val-OH |

Linking Groups Linking (spacer) groups are chemical moieties that link or connect reactive entities to antiviral or antifusogenic peptides. Linking groups may comprise one or more alkyl moeities, alkoxy moeity, alkenyl moeity, alkynyl moeity or amino moeity substituted by alkyl moeities, cycloalkyl moeity, polycyclic moeity, aryl moeity, polyaryl moeities, substituted aryl moeities, heterocyclic moeities, and substituted heterocyclic moeities. Linking groups may comprise (2-amino)ethoxy acetic acid (AEA), [2-(2-amino)ethoxy)]ethoxy acetic acid (AEEA), ethylenediamine (EDA); one or more alkyl chains (C1-C10) such as 8-aminooctanoic acid (AOA), 8-aminopropanoic acid (APA), or 4-aminobenzoic acid (APhA).

Sensitive Functional Groups: A sensitive functional group is a group of atoms that represents a potential reaction site on an antiviral and/or antifusogenic peptide. If present, a sensitive functional group may be chosen as the attachment point for the linker-reactive group modification. Sensitive functional groups include but are not limited to carboxyl, amino, thiol, and hydroxyl groups.

Modified Peptides: A modified peptide is an antiviral and/or antifusogenic peptide that has been modified by attaching a reactive group. The reactive group may be attached to the peptide either via a linking group, or optionally without using a linking group. It is also contemplated that one or more additional amino acids may be added to the peptide to facilitate the attachment of the reactive entity. Modified peptides may be administered in vivo such that conjugation with blood components occurs in vivo, or they may be first conjugated to blood components or carrier proteins in vitro (e.g., using recombinantly produced proteins, such as recombinant albumin, immunoglobulin, or transferring) and the resulting conjugated peptide (as defined below) administered in vivo.

Conjugated Peptides: A conjugated peptide is a modified peptide that has been conjugated to a blood component via a covalent bond formed between the reactive group of the modified peptide and the functionalities of the blood component, with or without a linking group. As used throughout this application, the term “conjugated peptide” can be made more specific to refer to particular conjugated peptides, for example “conjugated C34” or “conjugated DP107.”

In embodiments, the modified antiviral and/or antifusogenic peptides of the invention include a maleimide containing group which has the ability to covalently bond blood components and more particularly serum albumin so as to form a conjugate. The administration of a maleimide derivative of an antiviral and/or antifusogenic peptide to a subject can result in the in vivo conjugation of the peptide to a blood component such as serum albumin. It is also encompassed by the present invention to prepare the conjugate ex vivo (or in vivo) by contacting the modified antiviral and/or antifusogenic peptide with a blood component or carrier protein, e.g., albumin. In this case, albumin can be provided from different sources: in blood samples, purified albumin, recombinant albumin or the like. The preparation and use of conjugates of C34 and albumin have been thoroughly disclosed in WO 02/096935, and similar preparations and uses apply to conjugates of the present invention. The conjugates formed in vivo in a subject and the ex vivo prepared conjugates when administered to a subject are both useful for exhibiting antifusogenic activity of the corresponding fusion peptide inhibitor an, therefore, inhibiting the activity of HIV, RSV, HPV, MeV or SIV in a subject.

Taking into account these definitions, the present invention takes advantage of the properties of existing antiviral and antifusogenic peptides. The viruses that may be inhibited by the peptides include, but are not limited to all strains of viruses listed, e.g., in U.S. Pat. Nos. 6,013,263, 6,017,536 and 6,020,459 at Tables V-VII and IX-XIV therein. These viruses include, e.g., human retroviruses, including HIV-1, HIV-2, and human T-lympocyte viruses (HTLV-I and HTLV-II), and non-human retroviruses, including bovine leukosis virus, feline sarcoma virus, feline leukemia virus, simian immunodeficiency virus (SIV), simian sarcoma virus, simian leukemia, and sheep progress pneumonia virus. Non-retroviral viruses may also be inhibited by the peptides of the present invention, including human respiratory syncytial virus (RSV), canine distemper virus, Newcastle Disease virus, human parainfluenza virus (HPIV), influenza viruses, measles viruses (MeV), Epstein-Barr viruses, hepatitis B viruses, and simian Mason-Pfizer viruses. Non-enveloped viruses may also be inhibited by the peptides of the present invention, and include, but are not limited to, picornaviruses such as polio viruses, hepatitis A virus, enteroviruses, echoviruses, coxsackie viruses, papovaviruses such as papilloma virus, parvoviruses, adenoviruses, and reoviruses.

As an example, the mechanism of action of HIV fusion peptides has been described as discussed in the background section of this application and antiviral and antifusogenic properties of the peptides have been well established. A synthetic peptide corresponding to the carboxyl-terminal ectodomain sequence (for instance, amino acid residues 643-678 of HIV-1 class B, of the LAI strain or residues 638-673 from similar strain as well as residues 558-595) has been shown to inhibit virus-mediated cell-cell fusion completely at low concentration. The peptides of the invention compete with the leucine zipper region of the native viral gp41 thus resulting in the interference of the fusion/infection of the virus into the cell.

The invention additionally provides methods and reagents used to modify a selected antiviral and/or antifusogenic peptide with the DAC (Drug Activity Complex) technology to confer to this peptide improved bio-availability, extended half-life and better distribution through selective conjugation of the peptide onto a protein carrier but without modifying the peptide's antiviral properties. The carrier of choice (but not limited to) for this invention would be albumin conjugated through its free thiol by an antiviral and/or antifusogenic peptide modified with a maleimide moiety.

Antiviral and/or Antifusogenic Inhibitors

Several peptide sequences have been described in the literature as highly potent for the prevention of HIV-1 fusion/infection. As examples, peptides C34, DP107, DP178 binds to a conformation of gp41 that is relevant for fusion. Thus, in one embodiment of the invention, C34-, DP178- and DP178-like peptides are modified. Likewise, other embodiments of the invention include modification of C34-, DP107 and DP107-like peptide for use against HIV, as well as peptides analagous to DP107 and DP178 that are found in RSV, HPV, MeV and SIV viruses.

### Modified C34 Peptides or Analogues

Examples of modified C34 peptides that can be modified following the teachings of the application also include the following amino acid sequences:

(SEQ ID NO:5)

N term-W M E W D R E I N N Y T S L I H S L I E E S
Q N Q Q E K N E Q E L L-C term

(SEQ ID NO:6)

N term-W M E W D R E I N N Y T S L I H S L I E E S
Q N Q Q E R N E Q E L K-C term

(SEQ ID NO:7)

N term-W M E W D R E I N N Y T S L I H S L I E E S
Q N Q Q E R N E Q E K L-C term

(SEQ ID NO:8)

N term-W M E W D R E I N N Y T S L I H S L I E E S
Q N Q Q E R N E Q K L L-C term

(SEQ ID NO:9)

N term-W M E W D R E I N N Y T S L I H S L I E E S
Q N Q Q E R N E K E L L-C term

(SEQ ID NO:10)

N term-W M E W D R E I N N Y T S L I H S L I E E S
Q N Q Q E R N K Q E L L-C term

(SEQ ID NO:11)

N term-W M E W D R E I N N Y T S L I H S L I E E S
Q N Q Q E R K E Q E L L-C term

(SEQ ID NO:12)

N term-W M E W D R E I N N Y T S L I H S L I E E S
Q N Q Q E R N E Q E L L K-C term

(SEQ ID NO:13)

N term-W M E W D R E I N N Y T S L I H S L I E E S
Q N Q Q E K N E Q E L L K-C term

Non-limiting examples of modified C34 peptides are the compounds of Formulae I-VIII illustrated below, which are capable of reacting with thiol groups on a blood component either in vivo or ex vivo, to form a stable covalent bond. Synthesis of these compounds is described in WO 02/096935, the contents of which are hereby specifically incorporated by reference.

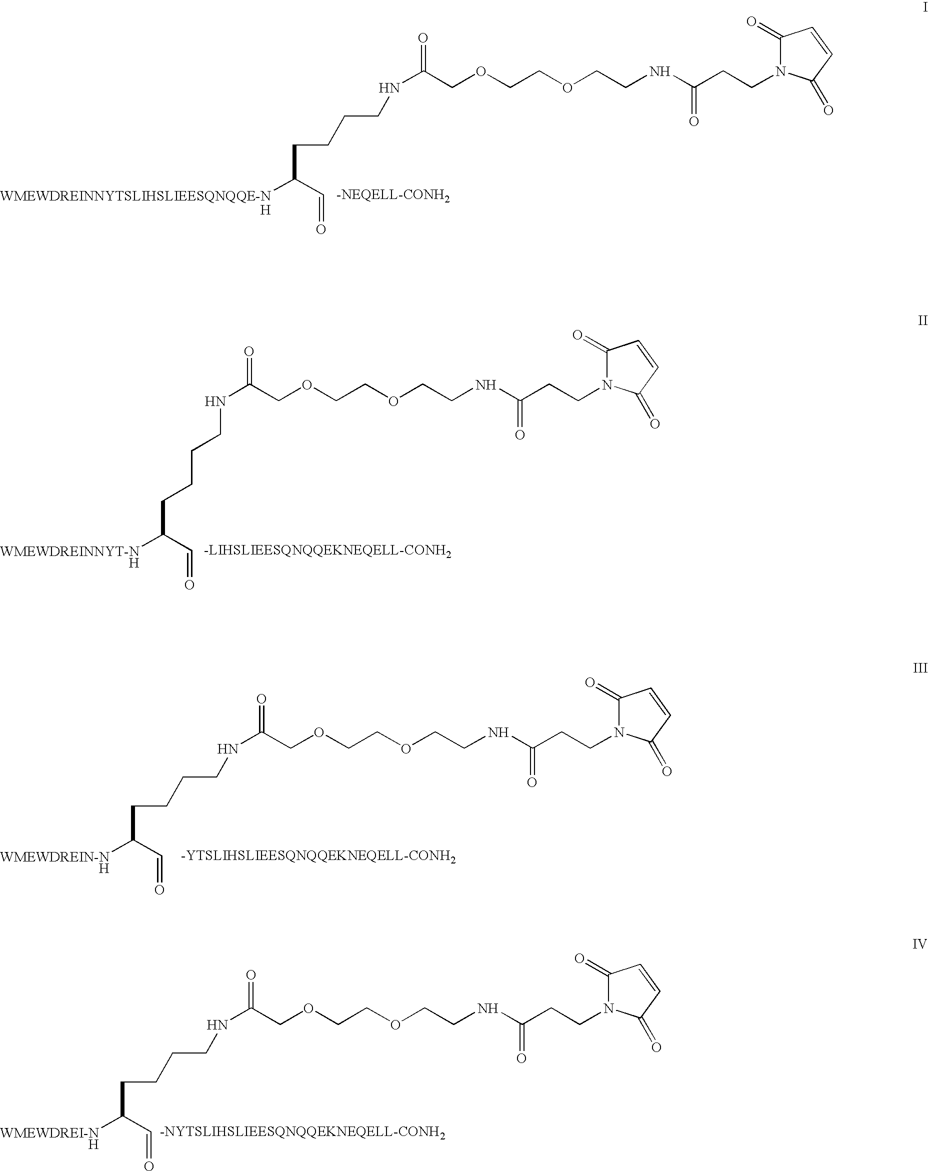

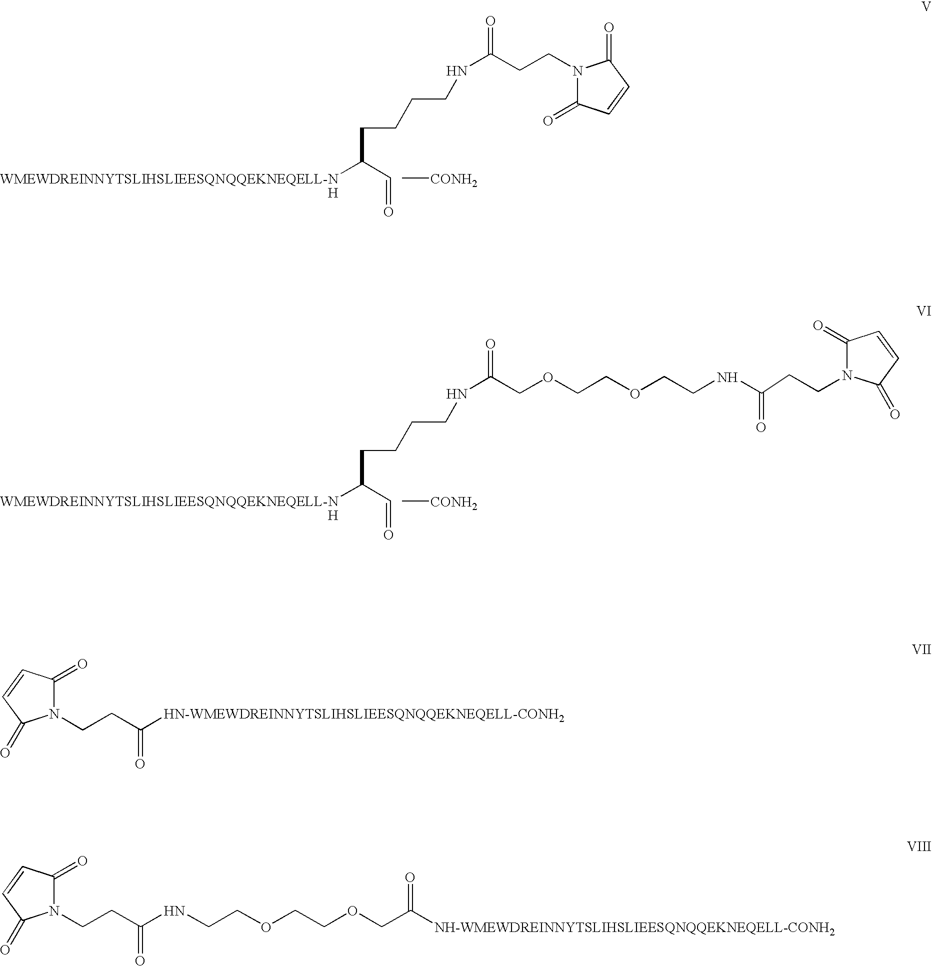

### DP178 and DP107

### DP178 Peptides

The DP178 peptide corresponds to amino acid residues 638 to 673 of the transmembrane protein gp41 from the HIV-1_{LAI} isolate, and has the 36 amino acid sequence (reading from amino to carboxy terminus):

(SEQ ID NO:14)

NH₂-YTSLIHSLIEESQNQQEKNEQELLELDKWASLWNWF-COOH

In addition to the full-length DP178 36-mer, the peptides of this invention include truncations of the DP178 peptide comprising peptides of between 3 and 36 amino acid residues (e.g., peptides ranging in size from a tripeptide to a 36-mer polypeptide), These truncated peptides are shown in Tables 2 and 3.

In addition amino acid substitutions of the DP178 peptide are also within the scope of the invention. HIV-1 and HIV-2 enveloped proteins are structurally distinct, but there exists a striking amino acid conservation within the DP178-corresponding regions of HIV-1 and HIV-2. The amino acid conservation is of a periodic nature, suggesting some conservation of structure and/or function. Therefore, one possible class of amino acid substitutions would include those amino acid changes which are predicted to stabilize the structure of the DP178 peptides of the invention. Utilizing the DP178 and DP178 analog sequences described herein, the skilled artisan can readily compile DP178 consensus sequences and ascertain from these, conserved amino acid residues which would represent preferred amino acid substitutions.

The amino acid substitutions may be of a conserved or non-conserved nature. Conserved amino acid substitutions consist of replacing one or more amino acids of the DP178 peptide sequence with amino acids of similar charge, size, and/or hydrophobicity characteristics, such as, for example, a glutamic acid (E) to aspartic acid (D) amino acid substitution. Non-conserved substitutions consist of replacing one or more amino acids of the DP178 peptide sequence with amino acids possessing dissimilar charge, size, and/or hydrophobicity characteristics, such as, for example, a glutamic acid (E) to valine (V) substitution.

Amino acid insertions of DP178 may consist of single amino acid residues or stretches of residues. The insertions may be made at the carboxy or amino terminal end of the DP178 or DP178 truncated peptides, as well as at a position internal to the peptide.

Such insertions will generally range from 2 to 15 amino acids in length. It is contemplated that insertions made at either the carboxy or amino terminus of the peptide of interest may be of a broader size range, with about 2 to about 50 amino acids being preferred. One or more such insertions may be introduced into DP178 or DP178 truncations, as long as such insertions result in peptides which may still be recognized by the 107×178×4, ALLMOTI5 or PLZIP search motifs described above.

Preferred amino or carboxy terminal insertions are peptides ranging from about 2 to about 50 amino acid residues in length, corresponding to gp41 protein regions either amino to or carboxy to the actual DP178 gp41 amino acid sequence, respectively. Thus, a preferred amino terminal or carboxy terminal amino acid insertion would contain gp41 amino acid sequences found immediately amino to or carboxy to the DP178 region of the gp41 protein.

Deletions of DP178 or DP178 truncations are also within the scope of this invention. Such deletions consist of the removal of one or more amino acids from the DPI 78 or DP178-like peptide sequence, with the lower limit length of the resulting peptide sequence being 4 to 6 amino acids.

Such deletions may involve a single contiguous or greater than one discrete portion of the peptide sequences. One or more such deletions may be introduced into DP178 or DP178 truncations, as long as such deletions result in peptides which may still be recognized by the 107×178×4, ALLMOTI5 or PLZIP search motifs described above.

### DP107 Peptides

DP107 is a 38 amino acid peptide which exhibits potent antiviral activity, and corresponds to residues 558 to 595 of HIV-1_{LAI} isolate transmembrane (TM) gp41 glycoprotein, as shown here:

(SEQ ID NO:4)

NH₂-NNLLRAIEAQQHLLQLTVWQIKQLQARILAVERYLKDQ-COOH

In addition to the full-length DP107 38-mer, the DP107 peptides include truncations of the DP107 peptide comprising peptides of between 3 and 38 amino acid residues (e.g., peptides ranging in size from a tripeptide to a 38-mer polypeptide). These peptides are shown in Tables 4 and 5 of US 2005/0070475.

In addition, amino acid substitutions of the DP178 peptide are also within the scope of the invention. As for DP178, there also exists a striking amino acid conservation within the DP107-corresponding regions of HIV-1 and HIV-2, again of a periodic nature, suggesting conservation of structure and/or function. Therefore, one possible class of amino acid substitutions includes those amino acid changes predicted to stabilize the structure of the DP107 peptides of the invention. Utilizing the DP107 and DP107 analog sequences described herein, the skilled artisan can readily compile DP107 consensus sequences and ascertain from these, conserved amino acid residues which would represent preferred amino acid substitutions.

The amino acid substitutions may be of a conserved or non-conserved nature. Conserved amino acid substitutions consist of replacing one or more amino acids of the DP107 peptide sequence with amino acids of similar charge, size, and/or hydrophobicity characteristics, such as, for example, a glutamic acid (E) to aspartic acid (D) amino acid substitution. Non-conserved substitutions consist of replacing one or more amino acids of the DP107 peptide sequence with amino acids possessing dissimilar charge, size, and/or hydrophobicity characteristics, such as, for example, a glutamic acid (E) to valine (V) substitution.

Amino acid insertions may consist of single amino acid residues or stretches of residues. The insertions may be made at the carboxy or amino terminal end of the DP107 or DP107 truncated peptides, as well as at a position internal to the peptide.

Such insertions will generally range from 2 to 15 amino acids in length. It is contemplated that insertions made at either the carboxy or amino terminus of the peptide of interest may be of a broader size range, with about 2 to about 50 amino acids being preferred. One or more such insertions may be introduced into DP107 or DP107 truncations, as long as such insertions result in peptides which may still be recognized by the 107×178×4, ALLMOTI5 or PLZIP search motifs described above.

Preferred amino or carboxy terminal insertions are peptides ranging from about 2 to about 50 amino acid residues in length, corresponding to gp41 protein regions either amino to or carboxy to the actual DP107 gp41 amino acid sequence, respectively. Thus, a preferred amino terminal or carboxy terminal amino acid insertion would contain gp41 amino acid sequences found immediately amino to or carboxy to the DP107 region of the gp41 protein.

Deletions of DP107 or DP107 truncations are also within the scope of this invention. Such deletions consist of the removal of one or more amino acids from the DP107 or DP107-like peptide sequence, with the lower limit length of the resulting peptide sequence being 4 to 6 amino acids.

Such deletions may involve a single contiguous or greater than one discrete portion of the peptide sequences. One or more such deletions may be introduced into DP107 or DP107 truncations, as long as such deletions result in peptides which may still be recognized by the 107×178×4, ALLMOTI5 or PLZIP search motifs.

DP107 and DP107 truncations are more fully described in U.S. Pat. No. 5,656,480.

### DP107 and DP178 Analogs

Peptides corresponding to analogs of the DP178, DP178 truncations, DP107 and DP107 truncation sequences of the invention, described, above, may be found in other viruses, including, for example, non-HIV-1 enveloped viruses, non-enveloped viruses and other non-viral organisms.

Such DP178 and DP107 analogs may, for example, correspond to peptide sequences present in transmembrane (“TM”) proteins of enveloped viruses and may, correspond to peptide sequences present in non enveloped and nonviral organisms. Such peptides may exhibit antifusogenic activity, antiviral activity, most particularly antiviral activity which is specific to the virus in which their native sequences are found, or may exhibit an ability to modulate intracellular processes involving coiled-coil peptide structures.

### DP178 Analogs

DP178 analogs are peptides whose amino acid sequences are comprised of the amino acid sequences of peptide regions of, for example, other (e.g., other than HIV-1) viruses that correspond to the gp41 peptide region from which DP178 was derived. Such viruses may include, but are not limited to, other HIV-1 isolates and HIV-2 isolates.

DP178 analogs derived from the corresponding gp41 peptide region of other (e.g., non HIV-1LAI) HIV-1 isolates may include, for example, peptide sequences as shown below.

(SEQ ID NO:15)

NH2-YTNTIYTLLEESQNQQEKNEQELLELDKWASLWNWF-COOH

(SEQ ID NO:16)

NH2-YTGIIYNLLEESQNQQEKNEQELLELDKWANLWNWF-COOH

(SEQ ID NO:17)

NH2-YTSLIYSLLEKSQIQQEKNEQELLELDKWASLWNWF-COOH

The peptides of SEQ ID NO:15, SEQ ID NO:16 and SEQ ID NO:17 are derived from HIV-1_{SF2}, HIV-1_{RF}, and HIV-1_{MN}, respectively. Other DP178 analogs include those derived from HIV-2, including the peptides of SEQ ID NO:18 and SEQ ID NO:19, which are derived from HIV-2_{ROD} and HIV-2_{NIHZ}, respectively. Still other useful analogs include the peptides of SEQ ID NO:20 and SEQ ID NO:21, which have been demonstrated to exhibit antiviral activity.

In the present invention, it is preferred that the DP178 analogs represent peptides whose amino acid sequences correspond to the DP178 region of the gp41 protein, it is also contemplated that the peptides disclosed herein may, additionally, include amino sequences, ranging from about 2 to about 50 amino acid residues in length, corresponding to gp41 protein regions either amino to or carboxy to the actual DP178 amino acid sequence.

Table 6 and Table 7 of US 2005/0070475 show some possible truncations of the HIV-2_{NIHZ} DP178 analog, which may comprise peptides of between 3 and 36 amino acid residues (e.g., peptides ranging in size from a tripeptide to a 36-mer polypeptide). Peptide sequences in these tables are listed from amino (left) to carboxy (right) terminus.

### Additional DP178 Analogs and DP107 Analogs

DP178 and DP107 analogs are recognized or identified, for example, by utilizing one or more of the 107×178×4, ALLMOTI5 or PLZIP computer-assisted search strategies described above. The search strategy identifies additional peptide regions which are predicted to have structural and/or amino acid sequence features similar to those of DP107 and/or DP178.

The search strategies are described fully in the example presented in Section 9 of U.S. Pat. Nos. 6,013,263, 6,017,536 and 6,020,459. While this search strategy is based, in part, on a primary amino acid motif deduced from DP107 and DP178, it is not based solely on searching for primary amino acid sequence homologies, as such protein sequence homologies exist within, but not between major groups of viruses. For example, primary amino acid sequence homology is high within the TM protein of different strains of HIV-1 or within the TM protein of different isolates of simian immunodeficiency virus (SIV).

The computer search strategy disclosed in U.S. Pat. Nos. 6,013,263, 6,017,536 and 6,020,459 successfully identified regions of proteins similar to DP107 or DP178. This search strategy was designed to be used with a commercially-available sequence database package, preferably PC/Gene.

In U.S. Pat. Nos. 6,013,263, 6,017,536 and 6,020,459, a series of search motifs, the 107×178×4, ALLMOTI5 and PLZIP motifs, were designed and engineered to range in stringency from strict to broad, with 107×178×4 being preferred. The sequences identified via such search motifs, such as those listed in Tables V-XIV, of U.S. Pat. Nos. 6,013,263, 6,017,536 and 6,020,459 potentially exhibit antifusogenic, such as antiviral, activity, may additionally be useful in the identification of antifusogenic, such as antiviral, compounds.

### Other Antiviral Peptides

### Anti-RSV Peptides

Anti-RSV peptides include DP178 and/or DP107 analogs identified from corresponding peptide sequences in RSV which have further been identified to inhibit viral infection by RSV. Such peptides of interest include the peptides of Table 16 and peptides of SEQ ID NO:10 to SEQ ID NO:30 of US 2005/0070475. Detailed protocols for synthesizing these peptides are disclosed in US 2005/0070475, the contents of which are hereby specifically incorporated by reference. Of particular interest are the following peptides:

| | |
|---|---|
| YTSVITIELSNIKENKCNGAKVKLIKQELDKYK | (SEQ ID NO:22) |
| TSVITIELSNIKENKCNGAKVKLIKQELDKYKN | (SEQ ID NO:23) |
| VITIELSNIKENKCNGAKVKLIKQELDKYKNAV | (SEQ ID NO:24) |
| IALLSTNKAVVSLSNGVSVLTSKVLDLKNYIDK | (SEQ ID NO:25) |

The peptide of SEQ ID NO:10 of US 2005/0070475 is derived from the F2 region of RSV and was identified in U.S. Pat. Nos. 6,103,236 and 6,020,459 using the search motifs described as corresponding to DP107 and DP178 peptides (e.g., “DP107/178 like”). The peptides of SEQ ID NO:14 to SEQ ID NO:16 of US 2005/0070475 each have amino acid sequences contained within the peptide of SEQ ID NO:10 of US 2005/0070475 and each has been shown to exhibit anti-RSV activity, in particular, inhibiting fusion and syncytia formation between RSV-infected and uninfected Hep-2 cells at concentrations of less than 50 μg/ml.

The peptide of SEQ ID NO:11 of US 2005/0070475 is derived from the F1 region of RSV and was identified in U.S. Pat. Nos. 6,103,236 and 6,020,459 using the search motifs described as corresponding to DP107 (e.g., “DP107-like”). The peptide of SEQ ID NO:29 of US 2005/0070475 contains amino acid sequences contained within the peptide of SEQ ID NO:10 of US 2005/0070475 and likewise has been shown to exhibit anti-RSV activity, in particular, inhibiting fusion and syncytia formation between RSV-infected and uninfected Hep-2 cells at concentrations of less than 50 μg/ml.

### Anti-HPIV Peptides

Anti-HPIV peptides include DP178 and/or DP107 analogs identified from corresponding peptide sequences in HPIV and which have further been identified to inhibit viral infection by HPIV. Such peptides of interest include the peptides of Table 17 and SEQ ID NO:31 to SEQ ID NO:62 of US 2005/0070475. Detailed protocols for synthesizing these peptides are disclosed in US 2005/0070475, the contents of which are hereby specifically incorporated by reference. Of particular interest are the following peptides:

| | |
|---|---|
| VEAKQARSDIEKLKEAIRDTNKAVQSVQSSIGNLI | (SEQ ID NO:26) |
| RSDIEKLKEAIRDTNKAVQSVQSSIGNLIVAIKSV | (SEQ ID NO:27) |
| NSVALDPIDISIELNKAKSDLEESKEWIRRSNQKL | (SEQ ID NO:28) |
| ALDPIDISIELNKAKSDLEESKEWIRRSNQKLDSI | (SEQ ID NO:29) |
| LDPIDISIELNKAKSDLEESKEWIRRSNQKLDSIG | (SEQ ID NO:30) |
| DPIDISIELNKAKSDLEESKEWIRRSNQKLDSIGN | (SEQ ID NO:31) |
| PIDISIELNKAKSDLEESKEWIRRSNQKLDSIGNW | (SEQ ID NO:32) |
| IDISIELNKAKSDLEESKEWIRRSNQKLDSIGNWH | (SEQ ID NO:33) |

The peptide of SEQ ID NO:31 of US 2005/0070475 is derived from the F1 region of HPIV-3 and was identified in U.S. Pat. Nos. 6,103,236 and 6,020,459 using the search motifs described as corresponding to DP107 (e.g., “DP107-like”). The peptides of SEQ ID NO:52 of US 2005/0070475 and SEQ ID NO:58 of US 2005/0070475 each have amino acid sequences contained within the peptide of SEQ ID NO:30 of US 2005/0070475 and each has been shown to exhibit anti-HPIV-3 activity, in particular, inhibiting fusion and syncytia formation between HPIV-3-infected Hep2 cells and uninfected CV-1W cells at concentrations of less than 1 μg/ml.

The peptide of SEQ ID NO:32 of US 2005/0070475 is also derived from the F1 region of HPIV-3 and was identified in U.S. Pat. Nos. 6,103,236 and 6,020,459 using the search motifs described as corresponding to DP178 (e.g., “DP178-like”). The peptides of SEQ ID NO:35 and SEQ ID NO:38 to SEQ ID NO:42 each of US 2005/0070475 have amino acid sequences contained within the peptide of SEQ ID NO:32 of US 2005/0070475 and each also has been shown to exhibit anti-HPIV-3 activity, in particular, inhibiting fusion and syncytia formation between HPIV-3-infected Hep2 cells and uninfected CV-1W cells at concentrations of less than 1 μg/ml.

### Anti-MeV Peptides

Anti-MeV peptides are DP178 and/or DP107 analogs identified from corresponding peptide sequences in measles virus (MeV) which have further been identified to inhibit viral infection by the measles virus. Such peptides of particular interest include the peptides of Table 19 and peptides of SEQ ID NO:74 to SEQ ID NO:86 of US 2005/0070475. Detailed protocols for synthesizing these peptides are disclosed in US 2005/0070475, the contents of which are hereby specifically incorporated by reference. Of particular interest are the peptides listed below.

| | |
|---|---|
| HRIDLGPPISLERLDVGTNLGNAIAKLEAKELLE | (SEQ ID NO:34) |
| IDLGPPISLERLDVGTNLGNAIAKLEAKELLESS | (SEQ ID NO:35) |
| LGPPISLERLDVGTNLGNAIAKLEAKELLESSDQ | (SEQ ID NO:36) |
| PISLERLDVGTNLGNAIAKLEAKELLESSDQILR | (SEQ ID NO:37) |

Sequences derived from measles virus were identified in U.S. Pat. Nos. 6,103,236 and 6,020,459 using the search motifs described as corresponding to DP178 (e.g., “DP178-like”). The peptides of SEQ ID NO:77, SEQ ID NO:79, SEQ ID NO:81 and SEQ ID NO:83 each of US 2005/0070475 have amino acid sequences so identified, and each has been shown to exhibit anti-MeV activity, in particular, inhibiting fusion and syncytia formation between MeV-infected Hep2 and uninfected Vero cells at concentrations of less than 1 μg/ml.

### Anti-SIV Peptides

Anti-SIV peptides are DP178 and/or DP107 analogs identified from corresponding peptide sequences in SIV which have further been identified to inhibit viral infection by SIV. Such peptides of interest include the peptides of Table 18 and peptides of SEQ ID NO:63 to SEQ ID NO:73 of US 2005/0070475. Detailed protocols for synthezing these peptides are disclosed in US 2005/0070475, the contents of which are hereby specifically incorporated by reference. Of particular interest are the following peptides:

| | |
|---|---|
| WQEWERKVDFLEENITALLEEAQIQQEKNMYELQK | (SEQ ID NO:38) |
| QEWERKVDFLEENITALLEEAQIQQEKNMYELQKL | (SEQ ID NO:39) |
| EWERKVDFLEENITALLEEAQIQQEKNMYELQKLN | (SEQ ID NO:40) |
| WERKVDFLEENITALLEEAQIQQEKNMYELQKLNS | (SEQ ID NO:41) |
| ERKVDFLEENITALLEEAQIQQEKNMYELQKLNSW | (SEQ ID NO:42) |
| RKVDFLEENITALLEEAQIQQEKNMYELQKLNSWD | (SEQ ID NO:43) |
| KVDFLEENITALLEEAQIQQEKNMYELQKLNSWDV | (SEQ ID NO:44) |
| VDFLEENITALLEEAQIQQEKNMYELQKLNSWDVF | (SEQ ID NO:45) |
| DFLEENITALLEEAQIQQEKNMYELQKLNSWDVFG | (SEQ ID NO:46) |
| FLEENITALLEEAQIQQEKNMYELQKLNSWDVFGN | (SEQ ID NO:47) |

Sequences derived from SIV transmembrane fusion protein were identified in U.S. Pat. Nos. 6,103,236 and 6,020,459 using the search motifs described as corresponding to DP178 (e.g., “DP178-like”). The peptides of SEQ ID NO:64 to SEQ ID NO:73 each of US 2005/0070475 have amino acid sequences so identified, and each has been shown to exhibit potent anti-SIV activity as crude peptides.

### Additional Viral and Fusion Inhibitors

The expression “viral inhibitor derivative” is intended to mean any modification or derivative of a viral inhibitor chosen from an antifusogenic compound or an entry Inhibitor (or non-antifusogenic) compound.

Antifusogenic compounds include, without limitation, enfuvirtide; C34; T-1249; TRI-899; TRI-999; 5-helix; N36 Mut (e.g); NCCG-gp41; DP-107; M41-P; N36; M87o; FM-006; ADS-J1; C14 linkmid; C34coil; hemolysin A; IQN17; IQN23; SC34EK; SPI-30,014; SPI-70,038; T-1249-HSA; T-649; T-651; TRI-1144; C14; MBP-107; scC34; SJ-2176; T-1249-transferrin; p26; p38; ADS-J2; C52L; clone 3 antibody; D5 IgG; D5 scFc; F240 scFv; sifuvirtide; IZN-36; T-1249 mimetibody; N-36-E; NB-2; NB-64; S-29-I; theaflavin-3,3′-digallate; VIRIP; siamycin I; siamycin II.

Entry Inhibitor (or non-antifusogenic) compounds include, without limitation, AMD-070; SPC-3; KRH-2731; AMD-8664; FC-131; HIV-1 Tat analogs; KRH-1120; KRH-1636; POL-2438; T-134; T-140; stromal cell-derived factor 1; ALX40-4C; AMD-3100; T-22; TJN-151; AM-1401; EradicAide viral macrophage inflammatory protein II; AMD-3451; conocurvone; maraviroc; vicriviroc; INCB-9471; INCB-15,050; DAPTA; PRO-140; HGS-004; SCH-C; TAK-652; TAK-220; nifeviroc; AMD-887; anti-CD63 MAb; AOP-RANTES; CPMD-167; E-913; FLSC R/T-IgG1; HGS-101; NIBR-1282; nonakine; PSC-RANTES; sCD4-17b; SCH-350,634; MIP-1 alpha; MIP-1 beta; RANTES; aplaviroc; peptide T; TAK-779; pCLXSN vector; UCB-35,625; J-113,863; CLIV; I-309; EGCG; Epigallocathechin Gallate; HB-19; lambda-carrageenan; PC-515; curdlan sulfate; OKU-40; OKU-41; VGV-1; Zintevir; AR-177; T-30,177; succinylated albumin; NSC1-658,586; ISIS-5320; RP-400c; SA-1042; C31G; Savvy; PRO-542; rCD4-IgG2; BMS-488,043; BMS-378,806; DES-6; 12 pl; Actinohivin; BlockAide/VP; CD4M33; CT-319; CT-326; cyanovirin-N; DCM-205; DES-10; griffithsin; HNG-105; NBD-556; NBD-557; PEG-cyanovirin-N; scytovirin; sCD4; dextrin-2-sulfate; F-105; FP-21,399; TNX-355; B4 MAb; R-15-K; sCD38(51-75) MBP; PRO-2000; NSC-13,778; SB-673,461M; SB-673,462M; rsCD4; Ac(Ala10,11) RANTES (2-14); IC-9564; RPR-103,611; Immudel-gp120; suligovir; IQP-0410; acetylated triiodothyronine; SP-01A; DEB-025; CSA-54; HGS-H/A 27; SP-10; VIR-5103; BMS-433,771; TMC-353,121; NSC-650,898; Michellamine B; NSC-692,906; TG-102; VIR-576; MEDI-488; CovX-Body; CNI-H0294.

### Modification of Antiviral and Antifusogenic Peptides

The invention contemplates modifying peptides that exhibit antiviral and/or antifusogenic activity, including such modifications of DP-107 and DP-178 and analogs thereof. Such modified peptides can react with the available reactive functionalities on blood components via covalent linkages. The invention also relates to such modifications, such combinations with blood components, and methods for their use. These methods include extending the effective therapeutic life of the conjugated antiviral peptides derivatives as compared to administration of the unconjugated peptides to a patient. The modified peptides are of a type designated as a DACT™ (Drug Affinity Complex) which comprises the antiviral peptide molecule and a linking group together with a chemically reactive group capable of reaction with a reactive functionality of a mobile blood protein. By reaction with the blood component or protein the modified peptide, or DAC, may be delivered via the blood to appropriate sites or receptors.

To form covalent bonds with functionalities on the protein, one may use as a reactive group a wide variety of active carboxyl groups, particularly esters, where the hydroxyl moiety is physiologically acceptable at the levels required to modify the peptide. While a number of different hydroxyl groups may be employed in these reactive groups, the most convenient would be N-hydroxysuccinimide or (NHS), N-hydroxy-sulfosuccinimide (sulfo-NHS). In preferred embodiments of this invention, the functionality on the protein will be a thiol group and the reactive group will be a maleimido-containing group such as gamma-maleimide-butyralamide (GMBA) or maleimidopropionic acid (MPA)

Primary amines are the principal targets for NHS esters. Accessible α-amine groups present on the N-termini of proteins react with NHS esters. However, α-amino groups on a protein may not be desirable or available for the NHS coupling. While five amino acids have nitrogen in their side chains, only the ε-amine of lysine reacts significantly with NHS esters. An amide bond is formed when the NHS ester conjugation reaction reacts with primary amines releasing N-hydroxysuccinimide as demonstrated in the schematic below.

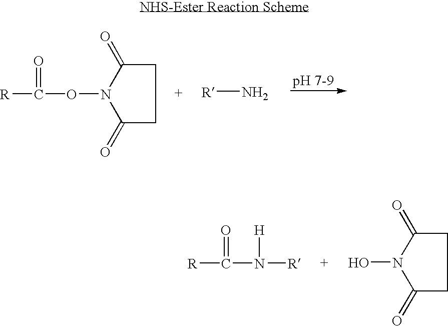

In the preferred embodiments of this invention, the functional group on this protein will be a thiol group and the chemically reactive group will be a maleimido-containing group such as MPA or GMBA (gamma-maleimide-butyralamide). The maleimido group is most selective for sulfhydryl groups on peptides when the pH of the reaction mixture is kept between 6.5 and 7.4. At pH 7.0, the rate of reaction of maleimido groups with sulfhydryls is 1000-fold faster than with amines. A stable thioether linkage between the maleimido group and the sulfhydryl is formed which cannot be cleaved under physiological conditions, as demonstrated in the following schematic.

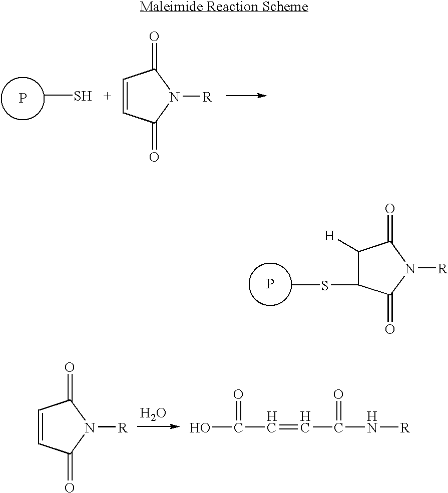

### Specific Labeling

Preferably, the modified peptides of this invention are designed to specifically react with thiol groups on mobile blood proteins. Such reaction is preferably established by covalent bonding of the peptide modified with a maleimide link (e.g. prepared from GMBS, MPA or other maleimides) to a thiol group on a mobile blood protein such as serum albumin or IgG.

Under certain circumstances, specific labeling with maleimides offers several advantages over non-specific labeling of mobile proteins with groups such as NHS and sulfo-NHS. Thiol groups are less abundant in vivo than amino groups. Therefore, the maleimide-modified peptides of this invention, e.g., maleimide peptides, will covalently bond to fewer proteins. For example, in albumin (the most abundant blood protein) there is only a single thiol group. Thus, peptide-maleimide-albumin conjugates will tend to comprise approximately a 1:1 molar ratio of peptide to albumin. In addition to albumin, IgG molecules (class II) also have free thiols. Since IgG molecules and serum albumin make up the majority of the soluble protein in blood they also make up the majority of the free thiol groups in blood that are available to covalently bond to maleimide-modified peptides.

Further, even among free thiol-containing blood proteins, including IgGs, specific labeling with maleimides leads to the preferential formation of peptide-maleimide-albumin conjugates, due to the unique characteristics of albumin itself. The single free thiol group of albumin, highly conserved among species, is located at amino acid residue 34 (Cys³⁴). It has been demonstrated recently that the Cys³⁴ of albumin has increased reactivity relative to free thiols on other free thiol-containing proteins. This is due in part to the very low pK value of 5.5 for the Cys³⁴ of albumin. This is much lower than typical pK values for cysteine residues in general, which are typically about 8. Due to this low pK, under normal physiological conditions Cys³⁴ of albumin is predominantly in the ionized form, which dramatically increases its reactivity. In addition to the low pK value of Cys³⁴, another factor which enhances the reactivity of Cys³⁴ is its location, which is in a crevice close to the surface of one loop of region V of albumin. This location makes Cys³⁴ very available to ligands of all kinds, and is an important factor in Cys³⁴'s biological role as free radical trap and free thiol scavenger. These properties make Cys³⁴ highly reactive with maleimide-peptides, and the reaction rate acceleration can be as much as 1000-fold relative to rates of reaction of maleimide-peptides with other free-thiol containing proteins.

Another advantage of peptide-maleimide-albumin conjugates is the reproducibility associated with the 1:1 loading of peptide to albumin specifically at Cys³⁴. Other techniques, such as glutaraldehyde, DCC, EDC and other chemical activations of, e.g, free amines, lack this selectivity. For example, albumin contains 52 lysine residues, 25-30 of which are located on the surface of albumin and therefore accessible for conjugation. Activating these lysine residues, or alternatively modifying peptides to couple through these lysine residues, results in a heterogenous population of conjugates. Even if 1:1 molar ratios of peptide to albumin are employed, the yield will consist of multiple conjugation products, some containing 0, 1, 2 or more peptides per albumin, and each having peptides randomly coupled at any one or more of the 25-30 available lysine sites. Given the numerous possible combinations, characterization of the exact composition and nature of each conjugate batch becomes difficult, and batch-to-batch reproducibility is all but impossible, making such conjugates less desirable as a therapeutic. Additionally, while it would seem that conjugation through lysine residues of albumin would at least have the advantage of delivering more therapeutic agent per albumin molecule, studies have shown that a 1:1 ratio of therapeutic agent to albumin is preferred. In an article by Stehle, et al., “The Loading Rate Determines Tumor Targeting properties of Methotrexate-Albumin Conjugates in Rats,” Anti-Cancer Drugs, Vol. 8, pp. 677-685 (1988), the authors report that a 1:1 ratio of the anti-cancer methotrexate to albumin conjugated via glutaraldehyde gave the most promising results. These conjugates were preferentially taken up by tumor cells, whereas conjugates bearing 5:1 to 20:1 methotrexate molecules had altered HPLC profiles and were quickly taken up by the liver in vivo. It is postulated that at these higher ratios, conformational changes to albumin diminish its effectiveness as a therapeutic carrier.

Through controlled administration of maleimide-peptides in vivo, one can control the specific labeling of albumin and IgG in vivo. In typical administrations, 80-90% of the administered maleimide-peptides will label albumin and less than 5% will label IgG. Trace labeling of free thiols such as glutathione will also occur. Such specific labeling is preferred for in vivo use as it permits an accurate calculation of the estimated half-life of the administered agent.

In addition to providing controlled specific in vivo labeling, maleimide-peptides can provide specific labeling of serum albumin and IgG ex vivo. Such ex vivo labeling involves the addition of maleimide-peptides to blood, serum or saline solution containing serum albumin and/or IgG. Once conjugation has occurred ex vivo with the maleimide-peptides, the blood, serum or saline solution can be readministered to the patient's blood for in vivo treatment.

In contrast to NHS-peptides, maleimide-peptides are generally quite stable in the presence of aqueous solutions and in the presence of free amines. Since maleimide-peptides will only react with free thiols, protective groups are generally not necessary to prevent the maleimide-peptides from reacting with itself. In addition, the increased stability of the modified peptide permits the use of further purification steps such as HPLC to prepare highly purified products suitable for in vivo use. Lastly, the increased chemical stability provides a product with a longer shelf life.

### Non-Specific Labeling

The antiviral peptides of the invention may also be modified for non-specific labeling of blood components. Bonds to amino groups will also be employed, particularly with the formation of amide bonds for non-specific labeling. To form such bonds, one may use as a chemically reactive group a wide variety of active carboxyl groups, particularly esters, where the hydroxyl moiety is physiologically acceptable at the levels required. While a number of different hydroxyl groups may be employed in these linking agents, the most convenient would be N-hydroxysuccinimide (NHS) and N-hydroxy-sulfosuccinimide (sulfo-NHS).

Other linking agents which may be utilized are described in U.S. Pat. No. 5,612,034.

The various sites with which the chemically reactive group of the modified peptides may react in vivo include cells, particularly red blood cells (erythrocytes) and platelets, and proteins, such as immunoglobulins, including IgG and IgM, serum albumin, ferritin, steroid binding proteins, transferrin, thyroxin binding protein, α-2-macroglobulin, and the like. Those receptors with which the modified peptides react, which are not long-lived, will generally be eliminated from the human host within about three days. The proteins indicated above (including the proteins of the cells) will remain at least three days, and may remain five days or more (usually not exceeding 60 days, more usually not exceeding 30 days) particularly as to the half life, based on the concentration in the blood.

For the most part, reaction will be with mobile components in the blood, particularly blood proteins and cells, more particularly blood proteins and erythrocytes. By “mobile” is intended that the component does not have a fixed situs for any extended period of time, generally not exceeding 5 minutes, more usually one minute, although some of the blood component may be relatively stationary for extended periods of time. Initially, there will be a relatively heterogeneous population of functionalized proteins and cells. However, for the most part, the population within a few days will vary substantially from the initial population, depending upon the half-life of the functionalized proteins in the blood stream. Therefore, usually within about three days or more, IgG will become the predominant functionalized protein in the blood stream.

Usually, by day 5 post-administration, IgG, serum albumin and erythrocytes will be at least about 60 mole %, usually at least about 75 mole %, of the conjugated components in blood, with IgG, IgM (to a substantially lesser extent) and serum albumin being at least about 50 mole %, usually at least about 75 mole %, more usually at least about 80 mole %, of the non-cellular conjugated components.

The desired conjugates of non-specific modified peptides to blood components may be prepared in vivo by administration of the modified peptides to the patient, which may be a human or other mammal. The administration may be done in the form of a bolus or introduced slowly over time by infusion using metered flow or the like.

If desired, the subject conjugates may also be prepared ex vivo by combining blood with modified peptides of the present invention, allowing covalent bonding of the modified peptides to reactive functionalities on blood components and then returning or administering the conjugated blood to the host. Moreover, the above may also be accomplished by first purifying an individual blood component or limited number of components, such as red blood cells, immunoglobulins, serum albumin, or the like, and combining the component or components ex vivo with the chemically reactive modified peptides. The functionalized blood or blood component may then be returned to the host to provide in vivo the subject therapeutically effective conjugates. The blood also may be treated to prevent coagulation during handling ex vivo.

### Synthesis of Modified Antiviral and Antifusogenic Peptides Peptide Synthesis

Antiviral and/or antifusogenic peptides according to the present invention may be synthesized by standard methods of solid phase peptide chemistry known to those of ordinary skill in the art. For example, peptides may be synthesized by solid phase chemistry techniques following the procedures described by Steward and Young (Steward, J. M. and Young, J. D., Solid Phase Peptide Synthesis, 2nd Ed., Pierce Chemical Company, Rockford, Ill., (1984) using an Applied Biosystem synthesizer. Similarly, multiple peptide fragments may be synthesized then linked together to form larger peptides. These synthetic peptides can also be made with amino acid substitutions at specific locations.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, Vol. 1, Academic Press (New York). In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth.

After all the desired amino acids have been linked in the proper sequence, any remaining protecting groups (and any solid support) are removed sequentially or concurrently to afford the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

A particularly preferred method of preparing compounds of the present invention involves solid phase peptide synthesis wherein the amino acid .alpha.-N-terminal is protected by an acid or base sensitive group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, .alpha.,.alpha.-dimethyl-3,5-dimethoxybenzyloxycarbonyl, o-nitrophenylsulfenyl, 2-cyano-t-butyloxycarbonyl, and the like. The 9-fluorenyl-methyloxycarbonyl (Fmoc) protecting group is particularly preferred for the synthesis of the peptides of the present invention. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxycarbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl(trityl).

In the solid phase peptide synthesis method, the .alpha.-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. The preferred solid support for synthesis of alpha.-C-terminal carboxy peptides is 4-hydroxymethylphenoxymethyl-copol-y(styrene-1% divinylbenzene). The preferred solid support for .alpha.-C-terminal amide peptides is the 4-(2′,4′-dimethoxyphenyl-Fmoc-am-inomethyl)phenoxyacetamidoethyl resin available from Applied Biosystems (Foster City, Calif.). The .alpha.-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetra-methyluronium-hexafluorophosphate (HBTU), with or without 4-dimethylaminopyridine (DMAP), 1-hydroxybenzotriazole (HOBT), benzotriazol-1-yloxy-tris(dimethylamino)phosphonium-hexafluorophosphate (BOP) or bis(2-oxo-3-oxazolidinyl)phosphine chloride (BOPCl), mediated coupling for from about 1 to about 24 hours at a temperature of between 10.degree. and 50.degree. C. in a solvent such as dichloromethane or DMF.

When the solid support is 4-(2′,4′-dimethoxyphenyl-Fmoc-aminomethyl-)phenoxy-acetamidoethyl resin, the Fmoc group is cleaved with a secondary amine, preferably piperidine, prior to coupling with the alpha.-C-terminal amino acid as described above. The preferred method for coupling to the deprotected 4-(2′,4′-dimethoxyphenyl-Fmoc-aminomethyl-)phenoxy-acetamidoethyl resin is O-benzotriazol-1-yl-N,N,N′,N′-tetramethyl-uroniumhexafluoro-phosphate (HBTU, 1 equiv.) and 1-hydroxybenzotriazole (HOBT, 1 equiv.) in DMF. The coupling of successive protected amino acids can be carried out in an automatic polypeptide synthesizer as is well known in the art. In a preferred embodiment, the alpha-N-terminal amino acids of the growing peptide chain are protected with Fmoc. The removal of the Fmoc protecting group from the alpha-N-terminal side of the growing peptide is accomplished by treatment with a secondary amine, preferably piperidine. Each protected amino acid is then introduced in about 3-fold molar excess, and the coupling is preferably carried out in DMF. The coupling agent is normally O-benzotriazol-1-yl-N,N,N′,N′-tetrame-thyluroniumhexafluorophosphate (HBTU, 1 equiv.) and 1-hydroxybenzotriazole (HOBT, 1 equiv.).

At the end of the solid phase synthesis, the polypeptide is removed from the resin and deprotected, either in successively or in a single operation. Removal of the polypeptide and deprotection can be accomplished in a single operation by treating the resin-bound polypeptide with a cleavage reagent comprising thioanisole, water, ethanedithiol and trifluoroacetic acid. In cases wherein the .alpha.-C-terminal of the polypeptide is an alkylamide, the resin is cleaved by aminolysis with an alkylamine. Alternatively, the peptide may be removed by transesterification, e.g. with methanol, followed by aminolysis or by direct transamidation. The protected peptide may be purified at this point or taken to the next step directly. The removal of the side chain protecting groups is accomplished using the cleavage cocktail described above. The fully deprotected peptide is purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing. Molecular weights of these ITPs are determined using Fast Atom Bombardment (FAB) Mass Spectroscopy.

### N-Terminal Protective Groups

As discussed above, the term “N-protecting group” refers to those groups intended to protect the alpha.-N-terminal of an amino acid or peptide or to otherwise protect the amino group of an amino acid or peptide against undesirable reactions during synthetic procedures. Commonly used N-protecting groups are disclosed in Greene, “Protective Groups In Organic Synthesis,” (John Wiley & Sons, New York (1981)), which is hereby incorporated by reference. Additionally, protecting groups can be used as pro-drugs which are readily cleaved in vivo, for example, by enzymatic hydrolysis, to release the biologically active parent.alpha.-N-protecting groups comprise lower alkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl-, 1-(p-biphenylyl)-1-methylethoxycarbonyl, .alpha.,.alpha.-dimethyl-3,5-di-methoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; arylalkyl groups such as benzyl, triphenylmethyl, benzyloxymethyl, 9-fluorenylmethyloxycarbonyl (Fmoc) and the like and silyl groups such as trimethylsilyl and the like.

### Carboxy Protective Groups

As discussed above, the term “carboxy protecting group” refers to a carboxylic acid protecting ester or amide group employed to block or protect the carboxylic acid functionality while the reactions involving other functional sites of the compound are performed. Carboxy protecting groups are disclosed in Greene, “Protective Groups in Organic Synthesis” pp. 152-186 (1981), which is hereby incorporated by reference. Additionally, a carboxy protecting group can be used as a pro-drug whereby the carboxy protecting group can be readily cleaved in vivo, for example by enzymatic hydrolysis, to release the biologically active parent. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated herein by reference. Representative carboxy protecting groups are C₁-C₈ loweralkyl (e.g., methyl, ethyl or t-butyl and the like); arylalkyl such as phenethyl or benzyl and substituted derivatives thereof such as alkoxybenzyl or nitrobenzyl groups and the like; arylalkenyl such as phenylethenyl and the like; aryl and substituted derivatives thereofsuch as 5-indanyl and the like; dialkylaminoalkyl such as dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl and the like; aroyloxyalkyl such as benzoyloxymethyl, benzoyloxyethyl and the like; arylalkylcarbonyloxyalkyl such as benzylcarbonyloxymethyl, 2-benzylcarbonyloxyethyl and the like; alkoxycarbonylalkyl or cycloalkyloxycarbonylalkyl such as methoxycarbonylmethyl, cyclohexyloxycarbonylmethyl, 1-methoxycarbonyl-1-ethyl and the like; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, 1-cyclohexyloxycarbonyloxy-1-ethyl and the like; aryloxycarbonyloxyalkyl such as 2-(phenoxycarbonyloxy)ethyl, 2-(5-indanyloxycarbonyloxy)ethyl and the like; alkoxyalkylcarbonyloxyalky-1 such as 2-(1-methoxy-2-methylpropan-2-oyloxy)ethyl and like; arylalkyloxycarbonyloxyalkyl such as 2-(benzyloxycarbonyloxy)ethyl and the like; arylalkenyloxycarbonyloxyalkyl such as 2-(3-phenylpropen-2-ylox-ylcarbonyloxy)ethyl and the like; alkoxycarbonylaminoalkyl such as t-butyloxycarbonylaminomethyl and the like; alkylaminocarbonylaminoalkyl such as methylaminocarbonylaminomethyl and the like; alkanoylaminoalkyl such as acetylaminomethyl and the like; heterocycliccarbonyloxyalkyl such as 4-methylpiperazinylcarbonyloxymethyl and the like; dialkylaminocarbonylalkyl such as dimethylaminocarbonylmethyl, diethylaminocarbonylmethyl and the like; (5-(loweralkyl)-2-oxo-1,3-dioxol-en-4-yl)alkyl such as (5-t-butyl-2-oxo-1,3-dioxolen-4-yl)methyl and the like; and (5-phenyl-2-oxo-1,3-dioxolen-4-yl)alkyl such as (5-phenyl-2-oxo-1,3-dioxolen-4-yl)methyl and the like.

Representative amide carboxy protecting groups are aminocarbonyl and lower alkylaminocarbonyl groups.

Preferred carboxy-protected compounds of the invention are compounds wherein the protected carboxy group is a loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester. Preferred amide carboxy protecting groups are lower alkylaminocarbonyl groups. For example, aspartic acid may be protected at the .alpha.-C-terminal by an acid labile group (e.g. t-butyl) and protected at the .beta.-C-terminal by a hydrogenation labile group (e.g. benzyl) then deprotected selectively during synthesis.

### Peptide Modification

The manner of producing the modified peptides of the present invention will vary widely, depending upon the nature of the various elements comprising the peptide. The synthetic procedures will be selected so as to be simple, provide for high yields, and allow for a highly purified stable product. Normally, the chemically reactive group will be created at the last stage of the synthesis, for example, with a carboxyl group, esterification to form an active ester. Specific methods for the production of modified peptides of the present invention are described below.

Specifically, the selected peptide is first assayed for antiviral activity, and then is modified with the linking group only at either the N-terminus, C-terminus or interior of the peptide. The antiviral activity of this modified peptide-linking group is then assayed. If the antiviral activity is not reduced dramatically (e.g., reduced less than 10-fold), then the stability of the modified peptide-linking group is measured by its in vivo lifetime. If the stability is not improved to a desired level, then the peptide is modified at an alternative site, and the procedure is repeated until a desired level of antiviral and stability is achieved.

More specifically, each peptide selected to undergo modification with a linker and a reactive entity group will be modified according to the following criteria: if a terminal carboxylic group is available on the peptide and is not critical for the retention of antiviral activity, and no other sensitive functional group is present on the peptide, then the carboxylic acid will be chosen as attachment point for the linker-reactive group modification. If the terminal carboxylic group is involved in antiviral activity, or if no carboxylic acids are available, then any other sensitive functional group not critical for the retention of antiviral activity will be selected as the attachment point for the linker-reactive entity modification. If several sensitive functional groups are available on a peptide, a combination of protecting groups will be used in such a way that after addition of the linker/reactive entity and deprotection of all the protected sensitive functional groups, retention of antiviral activity is still obtained. If no sensitive functional groups are available on the peptide, or if a simpler modification route is desired, synthetic efforts will allow for a modification of the original peptide in such a way that retention of antiviral is maintained. In this case the modification will occur at the opposite end of the peptide

An NHS derivative may be synthesized from a carboxylic acid in absence of other sensitive functional groups in the peptide. Specifically, such a peptide is reacted with N-hydroxysuccinimide in anhydrous CH₂Cl₂ and EDC, and the product is purified by chromatography or recrystallized from the appropriate solvent system to give the NHS derivative.

Alternatively, an NHS derivative may be synthesized from a peptide that contains an amino and/or thiol group and a carboxylic acid. When a free amino or thiol group is present in the molecule, it is preferable to protect these sensitive functional groups prior to perform the addition of the NHS derivative. For instance, if the molecule contains a free amino group, a transformation of the amine into aN Fmoc or preferably into a tBoc protected amine is necessary prior to perform the chemistry described above. The amine functionality will not be deprotected after preparation of the NHS derivative. Therefore this method applies only to a compound whose amine group is not required to be freed to induce the desired antiviral effect. If the amino group needs to be freed to retain the original properties of the molecule, then another type of chemistry described below has to be performed.

In addition, an NHS derivative may be synthesized from a peptide containing an amino or a thiol group and no carboxylic acid. When the selected molecule contains no carboxylic acid, an array of bifunctional linkers can be used to convert the molecule into a reactive NHS derivative. For instance, ethylene glycol-bis(succinimydylsuccinate) (EGS) and triethylamine dissolved in DMF and added to the free amino containing molecule (with a ratio of 10:1 in favor of EGS) will produce the mono NHS derivative. To produce an NHS derivative from a thiol derivatized molecule, one can use N-[-maleimidobutyryloxy]succinimide ester (GMBS) and triethylamine in DMF. The maleimido group will react with the free thiol and the NHS derivative will be purified from the reaction mixture by chromatography on silica or by HPLC.

An NHS derivative may also be synthesized from a peptide containing multiple sensitive functional groups. Each case will have to be analyzed and solved in a different manner. However, thanks to the large array of protecting groups and bifunctional linkers that are commercially available, this invention is applicable to any peptide with preferably one chemical step only to modify the peptide (as described above) or two steps (as described above involving prior protection of a sensitive group) or three steps (protection, activation and deprotection). Under exceptional circumstances only, would multiple steps (beyond three steps) synthesis be required to transform a peptide into an active NHS or maleimide derivative.

A maleimide derivative may also be synthesized from a peptide containing a free amino group and a free carboxylic acid. To produce a maleimide derivative from a amino derivatized molecule, one can use N-[.gamma.-maleimidobutyryloxy]succinimide ester (GMBS) and triethylamine in DMF. The succinimide ester group will react with the free amino and the maleimide derivative will be purified from the reaction mixture by crystallization or by chromatography on silica or by HPLC.

Finally, a maleimide derivative may be synthesized from a peptide containing multiple other sensitive functional groups and no free carboxylic acids. When the selected molecule contains no carboxylic acid, an array of bifunctional crosslinking reagents can be used to convert the molecule into a reactive NHS derivative. For instance maleimidopropionic acid (MPA) can be coupled to the free amine to produce a maleimide derivative through reaction of the free amine with the carboxylic group of MPA using HBTU/HOBt/DIEA activation in DMF.

Many other commercially available heterobifunctional crosslinking reagents can alternatively be used when needed. A large number of bifunctional compounds are available for linking to entities. Illustrative reagents include: azidobenzoyl hydrazide, N-[4-(p-azidosalicylamino)butyl]-3′-[2′-pyridyldithio)propionamide), bis-sulfosuccinimidyl suberate, dimethyl adipimidate, disuccinimidyl tartrate, N-.gamma.-maleimidobutyryloxysuccinimide ester, N-hydroxy sulfosuccinimidyl-4-azidobenzoate, N-succinimidyl [4-azidophenyl]-1,3′-di-thiopropionate, N-succinimidyl [4-iodoacetyl]aminobenzoate, glutaraldehyde, and succinimidyl 4-[N-maleimidomethyl]cyclohexane-1-carboxylate.

### Uses of Modified Antiviral Peptides

Modified antiviral peptides of the invention may be used as a therapeutic agent in the treatment of patients who are suffering from viral infection, and can be administered to patients according to the methods described below and other methods known in the art. Effective therapeutic dosages of the modified peptides may be determined through procedures well known by those in the art and will take into consideration any concerns over potential toxicity of the peptide.

The modified peptides can also be administered prophylactically to previously uninfected individuals. This can be advantageous in cases where an individual has been subjected to a high risk of exposure to a virus, as can occur when individual has been in contact with an infected individual where there is a high risk of viral transmission. This can be especially advantageous where there is known cure for the virus, such as the HIV virus. As a example, prophylactic administration of a modified anti-HIV peptide would be advantageous in a situation where a health care worker has been exposed to blood from an HIV-infected individual, or in other situations where an individual engaged in high-risk activities that potentially expose that individual to the HIV virus.

### Administration of Modified Antiviral and Antifusogenic Peptides

Generally, the modified peptides will be administered in a physiologically acceptable medium, e.g. deionized water, phosphate buffered saline (PBS), saline, aqueous ethanol or other alcohol, plasma, proteinaceous solutions, mannitol, aqueous glucose, alcohol, vegetable oil, or the like. Other additives which may be included include buffers, where the media are generally buffered at a pH in the range of about 5 to 10, where the buffer will generally range in concentration from about 50 to 250 mM, salt, where the concentration of salt will generally range from about 5 to 500 mM, physiologically acceptable stabilizers, and the like. The compositions may be lyophilized for convenient storage and transport.

The subject modified peptides will for the most part be administered parenterally, such as intravenously (IV), intraarterially (IA), intramuscularly (IM), subcutaneously (SC), or the like. Administration may in appropriate situations be by transfusion. In some instances, where reaction of the functional group is relatively slow, administration may be oral, nasal, rectal, transdermal or aerosol, where the nature of the conjugate allows for transfer to the vascular system. Usually a single injection will be employed although more than one injection may be used, if desired. The modified peptides may be administered by any convenient means, including syringe, trocar, catheter, or the like.

In certain embodiments, the modified peptides will be administered by pulmonary means by methods known in the art. Techniques for deep lung delivery of aerosol dry powder forms of peptides or proteins are disclosed by Patton et al. (1997) Chemtech 27(12):34-38. Additional references disclosing pulmonary administration of peptides include Senior, K. et al. (2000) PSTT Vol. 3:281-282; Gumbleton, M. (2006) Advanced Drug Delivery Reviews 58:993-995; Newhouse, M. T. (2006) Encyclopedia of Pharmaceutical Technology, entitled “Drug Delivery: Pulmonary Delivery;” and Labiris, N. R. (2003) J. Clin. Pharmacology 56:600-612. The contents of all of these references are hereby incorporated.

The particular manner of administration will vary depending upon the amount to be administered, whether a single bolus or continuous administration, or the like. Preferably, the administration will be intravascularly, where the site of introduction is not critical to this invention, preferably at a site where there is rapid blood flow, e.g., intravenously, peripheral or central vein. Other routes may find use where the administration is coupled with slow release techniques or a protective matrix. The intent is that the modified peptide be effectively distributed in the blood, so as to be able to react with the blood components. The concentration of the conjugate will vary widely, generally ranging from about 1 pg/ml to 50 mg/ml. The total administered intravascularly will generally be in the range of about 0.1 mg/ml to about 50 mg/ml, about 5 mg/ml to 40 mg/ml, about 10 to 30 mg/ml, about 10 to 20 mg/ml, or about 5 to 15 mg/ml, about 1 mg/ml to about 10 mg/ml, or about 1 to 5 mg/ml.

By bonding to long-lived components of the blood, such as immunoglobulin, serum albumin, red blood cells and platelets, a number of advantages ensue. The activity of the peptide is extended for days to weeks. Only one administration need be given during this period of time. Greater specificity can be achieved, since the active compound will be primarily bound to large molecules, where it is less likely to be taken up intracellularly to interfere with other physiological processes.

### Monitoring the Presence of Modified Peptides

The blood of the mammalian host may be monitored for the presence of the modified peptide compound one or more times. By taking a portion or sample of the blood of the host, one may determine whether the peptide has become bound to the long-lived blood components in sufficient amount to be therapeutically active and, thereafter, the level of the peptide compound in the blood. If desired, one may also determine to which of the blood components the peptide is bound. This is particularly important when using non-specific modified peptides. For specific maleimide-modified peptides, it is much simpler to calculate the half life of serum albumin and IgG.

### Immuno Assays

Another aspect of this invention relates to methods for determining the concentration of the antiviral peptides and/or analogs, or their derivatives and conjugates in biological samples (such as blood) using antibodies specific for the peptides, peptide analogs or their derivatives and conjugates, and to the use of such antibodies as a treatment for toxicity potentially associated with such peptides, analogs, and/or their derivatives or conjugates. This is advantageous because the increased stability and life of the peptides in vivo in the patient might lead to novel problems during treatment, including increased possibility for toxicity.

The use of anti-therapeutic agent antibodies, either monoclonal or polyclonal, having specificity for a particular peptide, peptide analog or derivative thereof, can assist in mediating any such problem. The antibody may be generated or derived from a host immunized with the particular peptide, analog or derivative thereof, or with an immunogenic fragment of the agent, or a synthesized immunogen corresponding to an antigenic determinant of the agent. Preferred antibodies will have high specificity and affinity for native, modified and conjugated forms of the peptide, peptide analog or derivative. Such antibodies can also be labeled with enzymes, fluorochromes, or radiolables.

Antibodies specific for modified peptides may be produced by using purified peptides for the induction of peptide-specific antibodies. By induction of antibodies, it is intended not only the stimulation of an immune response by injection into animals, but analogous steps in the production of synthetic antibodies or other specific binding molecules such as screening of recombinant immunoglobulin libraries. Both monoclonal and polyclonal antibodies can be produced by procedures well known in the art.

The anti-peptide antibodies may be used to treat toxicity induced by administration of the modified peptide, analog or derivative thereof, and may be used ex vivo or in vivo. Ex vivo methods would include immuno-dialysis treatment for toxicity employing anti-therapeutic agent antibodies fixed to solid supports. In vivo methods include administration of anti-therapeutic agent antibodies in amounts effective to induce clearance of antibody-agent complexes.

The antibodies may be used to remove the modified peptides, analogs or derivatives thereof, and conjugates thereof, from a patient's blood ex vivo by contacting the blood with the antibodies under sterile conditions. For example, the antibodies can be fixed or otherwise immobilized on a column matrix and the patient's blood can be removed from the patient and passed over the matrix. The modified peptide, peptide analogs, derivatives or conjugates will bind to the antibodies and the blood containing a low concentration of peptide, analog, derivative or conjugate, then may be returned to the patient's circulatory system. The amount of peptide compound removed can be controlled by adjusting the pressure and flow rate.

Preferential removal of the peptides, analogs, derivatives and conjugates from the plasma component of a patient's blood can be effected, for example, by the use of a semipermeable membrane, or by otherwise first separating the plasma component from the cellular component by ways known in the art prior to passing the plasma component over a matrix containing the anti-therapeutic antibodies. Alternatively the preferential removal of peptide-conjugated blood cells, including red blood cells, can be effected by collecting and concentrating the blood cells in the patient's blood and contacting those cells with fixed anti-therapeutic antibodies to the exclusion of the serum component of the patient's blood.

The anti-therapeutic antibodies can be administered in vivo, parenterally, to a patient that has received the peptide, analogs, derivatives or conjugates for treatment. The antibodies will bind peptide compounds and conjugates. Once bound the peptide activity will be hindered if not completely blocked thereby reducing the biologically effective concentration of peptide compound in the patient's bloodstream and minimizing harmful side effects. In addition, the bound antibody-peptide complex will facilitate clearance of the peptide compounds and conjugates from the patient's blood stream.

The invention having been fully described can be further appreciated and understood with reference to the following non-limiting examples.

The expression “viral inhibitor derivative” is intended to mean any modification or derivative of a viral inhibitor chosen from an antifusogenic compound or an entry Inhibitor (or non-antifusogenic) compound.

Antifusogenic compounds include, without limitation, enfuvirtide; C34; T-1249; TRI-899; TRI-999; 5-helix; N36 Mut (e.g); NCCG-gp41; DP-107; M41-P; N36; M87o; FM-006; ADS-J1; C14 linkmid; C34coil; hemolysin A; IQN17; IQN23; SC34EK; SPI-30,014; SPI-70,038; T-1249-HSA; T-649; T-651; TRI-1144; C14; MBP-107; scC34; SJ-2176; T-1249-transferrin; p26; p38; ADS-J2; C52L; clone 3 antibody; D5 IgG; D5 scFc; F240 scFv; sifuvirtide; IZN-36; T-1249 mimetibody; N-36-E; NB-2; NB-64; S-29-I; theaflavin-3,3′-digallate; VIRIP; siamycin I; siamycin II.

Entry Inhibitor (or non-antifusogenic) compounds include, without limitation, AMD-070; SPC-3; KRH-2731; AMD-8664; FC-131; HIV-1 Tat analogs; KRH-1120; KRH-1636; POL-2438; T-134; T-140; stromal cell-derived factor 1; ALX40-4C; AMD-3100; T-22; TJN-151; AM-1401; EradicAide viral macrophage inflammatory protein II; AMD-3451; conocurvone; maraviroc; vicriviroc; INCB-9471; INCB-15,050; DAPTA; PRO-140; HGS-004; SCH-C; TAK-652; TAK-220; nifeviroc; AMD-887; anti-CD63 MAb; AOP-RANTES; CPMD-167; E-913; FLSC R/T-IgG1; HGS-101; NIBR-1282; nonakine; PSC-RANTES; sCD4-17b; SCH-350,634; MIP-1 alpha; MIP-1 beta; RANTES; aplaviroc; peptide T; TAK-779; pCLXSN vector; UCB-35,625; J-113,863; CLIV; I-309; EGCG; Epigallocathechin Gallate; HB-19; lambda-carrageenan; PC-515; curdlan sulfate; OKU-40; OKU-41; VGV-1; Zintevir; AR-177; T-30,177; succinylated albumin; NSC1-658,586; ISIS-5320; RP-400c; SA-1042; C31G; Savvy; PRO-542; rCD4-IgG2; BMS-488,043; BMS-378,806; DES-6; 12pl; Actinohivin; BlockAide/VP; CD4M33; CT-319; CT-326; cyanovirin-N; DCM-205; DES-10; griffithsin; HNG-105; NBD-556; NBD-557; PEG-cyanovirin-N; scytovirin; sCD4; dextrin-2-sulfate; F-105; FP-21,399; TNX-355; B4 MAb; R-15-K; sCD38(51-75) MBP; PRO-2000; NSC-13,778; SB-673,461M; SB-673,462M; rsCD4; Ac(Ala10,11) RANTES (2-14); IC-9564; RPR-103,611; Immudel-gp120; suligovir; IQP-0410; acetylated triiodothyronine; SP-01A; DEB-025; CSA-54; HGS-H/A 27; SP-10; VIR-5103; BMS-433,771; TMC-353,121; NSC-650,898; Michellamine B; NSC-692,906; TG-102; VIR-576; MEDI-488; CovX-Body; CNI-H0294.

These viral inhibitor derivatives provide for an increased stability in vivo and a reduced susceptibility to peptidase or protease degradation.

Preferably, the viral inhibitor derivatives are conjugated with a blood component, e.g., serum albumin, e.g., human serum albumin, by covalent bond through a compound. In some embodiments, the blood component is bound to the viral inhibitor by a compound of Formulae I-V. viral inhibitor derivatives, e.g., described herein, minimize the need for more frequent, or even continual, administration of the peptides. The present viral inhibitor derivatives can be used, e.g., as a prophylactic against and/or treatment for infection of a number of viruses, including human immunodeficiency virus (HIV), human respiratory syncytial virus (RSV), human parainfluenza virus (HPV), measles virus (MeV) and simian immunodeficiency virus (SIV).

The modification made to the viral inhibitor allows it to react with available thiol groups on blood components to form stable covalent bonds. In one embodiment of the invention, the blood component comprises a blood protein, including a mobile blood protein such as albumin, which is most preferred.

The antifusogenic derivatives inhibit viral infection of cells, by, for example, inhibiting cell-cell fusion or free virus infection. The route of infection may involve membrane fusion, as occurs in the case of enveloped or encapsulated viruses, or some other fusion event involving viral and cellular structures.

The blood components to which the present viral inhibitor derivatives covalently bond may be either fixed or mobile. Fixed blood components are non-mobile blood components and may include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5 minutes, and more usually one minute. These blood components are not membrane-associated and are present in the blood for extended periods of time in a minimum concentration of at least 0.1 μg/ml. Mobile blood components include serum albumin, transferrin, ferritin and immunoglobulins such as IgM and IgG. The half-life of mobile blood components is at least about 12 hours in humans.

The viral inhibitor derivatives may be administered in vivo such that conjugation with blood components occurs in vivo, or they may be first conjugated to blood components of recombinant or genomic source in vitro and the resulting conjugated derivative administered in vivo.

The present invention takes advantage of the properties of existing antiviral, entry and antifusogenic inhibitors. The viruses that may be inhibited by the viral inhibitor include, but are not limited to all strains of viruses listed, e.g., in U.S. Pat. No. 6,013,263 and U.S. Pat. No. 6,017,536 at Tables V-VII and IX-XIV therein. These viruses include, e.g., human retroviruses, including HIV-1, HIV-2, and human T-lymphocyte viruses (HTLV-I and HTLV-II), and non-human retroviruses, including bovine leukosis virus, feline sarcoma virus, feline leukemia virus, simian immunodeficiency virus (SIV), simian sarcoma virus, simian leukemia, and sheep progress pneumonia virus. Non-retroviral viruses may also be inhibited by the C34 peptide derivatives, including human respiratory syncytial virus (RSV), canine distemper virus, Newcastle Disease virus, human parainfluenza virus (HPIV), influenza viruses, measles viruses (MeV), Epstein-Barr viruses, hepatitis B viruses, and simian Mason-Pfizer viruses. Non-enveloped viruses may also be inhibited by the viral inhibitor derivatives, and include, but are not limited to, picornaviruses such as polio viruses, hepatitis A virus, enteroviruses, echoviruses, coxsackie viruses, papovaviruses such as papilloma virus, parvoviruses, adenoviruses, and reoviruses.

The C34 peptide derivatives described herein can be designed to specifically react with thiol groups on mobile blood proteins. Such reaction is established by covalent bonding of the peptide modified with a maleimide link to a thiol group on a mobile blood protein such as serum albumin or IgG.

Thiol groups being less abundant in vivo than, for example, amino groups, the maleimide-modified C34 peptide, e.g., as described herein, will covalently bond to fewer proteins. For example, in albumin (the most abundant blood protein) there is only a single thiol group. Thus, a C34-maleimide-albumin conjugate will tend to comprise approximately a 1:1 molar ratio of C34 peptide to albumin. In addition to albumin, IgG molecules (class II) also have free thiols. Since IgG molecules and serum albumin make up the majority of the soluble protein in blood they also make up the majority of the free thiol groups in blood that are available to covalently bond to the C34 peptide derivative.

Further, even among free thiol-containing blood proteins, including IgGs, specific labeling with a maleimide leads to the preferential formation of a C34-maleimide-albumin conjugate due to the unique characteristics of albumin itself. The single free thiol group of albumin, highly conserved among species, is located at amino acid residue 34 (Cysteine-34). It has been demonstrated recently that the Cysteine-34 of albumin has increased reactivity relative to free thiols on other free thiol-containing proteins. This is due in part to the very low pK value of 5.5 for the Cysteine-34 of albumin. This is much lower than typical pK values for cysteine residues in general, which are typically about 8. Due to this low pK, under normal physiological conditions Cysteine-34 of albumin is predominantly in the ionized form, which dramatically increases its reactivity. In addition to the low pK value of Cysteine-34, another factor which enhances the reactivity of Cysteine-34 is its location, which is in a hydrophobic pocket close to the surface of one loop of region V of albumin. This location makes Cysteine-34 very available to ligands of all kinds, and is an important factor in Cysteine-34's biological role as free radical trap and free thiol scavenger. These properties make Cysteine-34 highly reactive with maleimide-C34

Another advantage of C34-maleimide-albumin conjugates is the reproducibility associated with the 1:1 loading of C34 to albumin specifically at Cysteine-34. Other techniques, such as glutaraldehyde, DCC, EDC and other chemical activations of, e.g, free amines, lack this selectivity. For example, albumin contains 52 lysine residues, 25-30 of which are located on the surface of albumin and therefore accessible for conjugation. Activating these lysine residues, or alternatively modifying C34 to couple through these lysine residues, results in a heterogenous population of conjugates. Even if 1:1 molar ratios of C34 to albumin are employed, the yield will consist of multiple conjugation products, some containing 0, 1, 2 or more C34 per albumin, and each having C34 randomly coupled at any one or more of the 25-30 available lysine sites. Given the numerous possible combinations, characterization of the exact composition and nature of each conjugate batch becomes difficult, and batch-to-batch reproducibility is all but impossible, making such conjugates less desirable as a therapeutic. Additionally, while it would seem that conjugation through lysine residues of albumin would at least have the advantage of delivering more therapeutic agent per albumin molecule, studies have shown that a 1:1 ratio of therapeutic agent to albumin is preferred. See, e.g., Stehle, et al., “The Loading Rate Determines Tumor Targeting properties of Methotrexate-Albumin Conjugates in Rats,” Anti-Cancer Drugs, Vol. 8, pp. 677-685 (1988), incorporated herein in its entirety.

Through controlled administration of viral inhibitor derivatives, such as those described herein, in vivo, one can control the specific labeling of albumin and IgG in vivo. In typical administrations, 80-90% of the administered viral inhibitor derivatives will label albumin and less than 5% will label IgG. Trace labeling of free thiols such as glutathione will also occur. Such specific labeling is preferred for in vivo use as it permits an accurate calculation of the estimated half-life of viral inhibitor.

The C34 peptide can be conjugated to a blood component by a lysine residue that occurs in C34 or can be conjugated at a site added, e.g., through peptide synthesis.

In addition to providing controlled specific in vivo labeling, the C34 peptide derivatives can provide specific labeling of serum albumin and IgG ex vivo. Such ex vivo labeling involves the addition of the C34 derivatives to blood, serum or saline solution containing serum albumin and/or IgG. Once conjugation has occurred ex vivo with the C34 derivative, the blood, serum or saline solution can be readministered to the patient's blood for in vivo treatment, or lyophilized.

A C34 derivative can be synthesized by standard methods of solid phase peptide chemistry well known to any one of ordinary skill in the art. For example, the peptide may be synthesized by solid phase chemistry techniques following the procedures described by Steward et al. in Solid Phase Peptide Synthesis, 2nd Ed., Pierce Chemical Company, Rockford, Ill., (1984) using a Rainin PTI Symphony synthesizer. Similarly, peptides fragments may be synthesized and subsequently combined or linked together to form the C34 peptide sequence (segment condensation).

For solid phase peptide synthesis, a summary of the many techniques may be found in Stewart et al. in “Solid Phase Peptide Synthesis”, W. H. Freeman Co. (San Francisco), 1963 and Meienhofer, Hormonal Proteins and Peptides, 1973, 246. For classical solution synthesis, see for example Schroder et al. in “The Peptides”, volume 1, Academic Press (New York). In general, such method comprise the sequential addition of one or more amino acids or suitably protected amino acids to a growing peptide chain on a polymer. Normally, either the amino or carboxyl group of the first amino acid is protected by a suitable protecting group. The protected and/or derivatized amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth.

After all the desired amino acids have been linked in the proper sequence, any remaining protecting groups (and any solid support) are cleaved sequentially or concurrently to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

The following examples are provided to illustrate preferred embodiments of the invention and shall by no means be construed as limiting its scope.

The viral inhibitor derivatives can be administered to patients according to the methods described below and other methods known in the art. Effective therapeutic dosages of the viral inhibitor derivatives can be determined through procedures well known by those in the art and will take into consideration any concerns over potential toxicity of viral inhibitor.

The viral inhibitor derivative can also be administered prophylactically to previously uninfected individuals. This can be advantageous in cases where an individual has been subjected to a high risk of exposure to a virus, as can occur when individual has been in contact with an infected individual where there is a high risk of viral transmission. This can be especially advantageous where there is no known cure for the virus, such as the HIV virus. As an example, prophylactic administration of a viral inhibitor derivative would be advantageous in a situation where a health care worker has been exposed to blood from an HIV-infected individual, or in other situations where an individual engaged in high-risk activities that potentially expose that individual to the HIV virus.

The invention having been fully described can be further appreciated and understood with reference to the following non-limiting examples.

### EXAMPLES

### Example 1

### Comparison of the Antiviral Activity of a Single Pre-Exposure Dose of Albumin-Conjugated Compound VIII, and Truvada™ Against HIV-1 NL4-3 in SCID-hu Thy/Liv Mice

Albumin-conjugated Compound VIII, an albumin-conjugated peptide fusion inhibitor was modeled on the fusion inhibitor C34 (C34). It was designed to facilitate less frequent dosing in humans by increasing in vivo half-life (>10 days) and sustaining plasma levels compared with unconjugated peptide. The drug is a 1:1 covalent conjugate with specific attachment of the fusion-inhibiting peptide on cysteine 34 of albumin. The peptide is predicted to bind to the N-heptad repeat of gp41. The activity of a single pre-exposure dose of albumin-conjugated Compound VIII, was evaluated in SCID-hu Thy/Liv mice infected with HIV-1 NL4-3 24 hours after subcutaneous administration of one large dose of the C34 derivative of the present invention albumin-conjugated Compound VIII (60 or 200 mg/kg calculated amount for Compound VIII peptide and not entire PC molecule). Thy/Liv implants were inoculated with 1,000 TCID50 of HIV-1 by direct injection and were collected 3 weeks later. The implants were dispersed into single-cell suspensions and assessed for p24 by ELISA, for HIV-1 RNA by the branched DNA assay, and for depletion of thymocyte subsets by multiparameter flow cytometry.

The usual dosing regimen for this mouse model when testing the anti-HIV activities of T-20 and other compounds is twice daily s.c. injections or oral gavage over 21 days (e.g. the entire length of the study). As can be noted in the p24 and HIV-1 RNA columns, albumin-conjugated Compound VIII exhibits very potent antiviral activity (as compared to NL4-3 untreated control) following only one strong dose 24 hr prior to infection as compared to Truvada (Gilead) which fails to exert any appreciable activity. Truvada is a small molecule inhibitor dosed orally, therefore 200 mg/kg of Truvada represents far more number of moles of drug than that for albumin-conjugated Compound VIII owing to Compound VIII's relatively large Mw (e.g. Mw>4 kDa).

TABLE 2
Protocol

| | |
|---|---|
| Implantation date | Jun. 17, 2006 |
| Implant age | 19 weeks |
| Donor ID | #061606 |
| Inoculation date | Oct. 31, 2006 |
| Virus | HIV-1 NL4-3; batch lipo IV (diluted 1:3) |
| Inoculum | 1,000 TCID₅₀ per implant |
| Termination date | Nov. 21, 2006 (21 days after inoculation) |
| Drugs | albumin-conjugated Compound VIII), [ConjuChem, |
| | Montreal, lot #10.23.06 (69 mg/ml) and |
| | lot #09.29.06 (230 mg/ml)] Truvada |
| | (Gilead, 300 mg tenofovir DF/200 mg |
| | (−)-FTC per tablet, lot #FDC046) |
| Vehicle | 8 mM NA octanoate in 1.5% polysorbate 80 |
| | (dosing solution prepared by ConjuChem) sterile |
| | water for Truvada (Aquaject, prod. #301-9915, |
| | lot #409355F) |
| Route | subcutaneous albumin-conjugated Compound VIII, |
| | oral gavage for Truvada |
| Dosing | Once |
| Volume | 400 μl for albumin-conjugated Compound VIII, |
| | 500 μl for Truvada |
| Treatment initiation | 1 day before virus inoculation |

TABLE 3
Results

| | | | | | | | HIV-1 | | |
|---|---|---|---|---|---|---|---|---|---|
| | | | | | | | RNA | FACS analysis | |
| | | | | | p24 | | (log₁₀ | Gag-p24⁺ | |
| | Mice/ | | | Dose | (pg/10⁶ | (% of | copies/ | thymocytes | CD4⁺CD8⁺ |
| Group | group | Virus | Drug | (mg/kg) | cells) | control) | 10⁶ cells) | (%) | (%) |
| A | 7 | NL4-3 | albumin- | 60 (900 | 36 ± 12* | 5.2 ± 1.7 | 4.1 ± 0.41* | 0.25 ± 0.09* | 82 ± 1.6* |
| | | | conjugated | total) | | | | | |
| | | | Compound | | | | | | |
| | | | VIII | | | | | | |
| B | 6 | NL4-3 | albumin- | 200 (3,000 | 28 ± 25* | 4.0 ± 3.6 | 2.8 ± 0.49* | 0.13 ± 0.08* | 83 ± 1.1* |
| | | | conjugated | total) | | | | | |
| | | | Compound | | | | | | |
| | | | VIII | | | | | | |
| C | 6 | NL4-3 | Truvada | 60/40 | 750 ± 280 | 110 ± 42 | 5.8 ± 0.25 | 7.9 ± 1.4 | 64 ± 2.2 |
| | | | | (TDF/FTC) | | | | | |
| D | 7 | NL4-3 | Truvada | 200/130 | 280 ± 64* | 41 ± 9.3 | 5.3 ± 0.14* | 3.1 ± 0.87* | 75 ± 1.8* |
| | | | | (TDF/FTC) | | | | | |
| E^{†} | 7 | NL4-3 | — | untreated | 680 ± 130 | 100 ± 19 | 6.1 ± 0.18 | 7.9 ± 0.69 | 59 ± 5.3 |
| F | 4 | medium | — | untreated | negative* | 0.0 ± 0.0 | negative* | 0.03 ± 0.01* | 78 ± 3.5* |

| | FACS analysis | | | | | | | |
|---|---|---|---|---|---|---|---|---|
| | | | | W6/32 | | | Live | Body |
| | | | | mean | Live | Total | thymocyte | weight |
| | CD4⁺ | CD8⁺ | CD4/CD8 | fluorescence | hymocytes | cell yield | yield | change |
| Group | (%) | (%) | ratio | intensity | (%) | (10⁶) | (10⁶) | (%) |
| A | 7.4 ± 0.52* | 5.0 ± 0.73* | 1.6 ± 0.09* | 610 ± 81* | 79 ± 2.2* | 250 ± 62* | 200 ± 53* | −0.61 |
| B | 6.8 ± 0.41* | 4.2 ± 0.39* | 1.6 ± 0.08* | 450 ± 42* | 80 ± 0.84* | 260 ± 52* | 210 ± 41* | −0.53 |
| C | 14 ± 0.89 | 12 ± 1.0 | 1.2 ± 0.08 | 2700 ± 440 | 58 ± 2.5 | 75 ± 14 | 44 ± 8.7 | +1.1 |
| D | 9.3 ± 0.62* | 7.2 ± 0.72* | 1.3 ± 0.06* | 2200 ± 100 | 67 ± 2.1 | 130 ± 20 | 88 ± 14 | +0.49 |
| E^{†} | 15 ± 1.5 | 13 ± 1.3 | 1.1 ± 0.02 | 2400 ± 430 | 57 ± 9.0 | 72 ± 23 | 47 ± 17 | +1.1 |
| F | 8.8 ± 1.8* | 7.1 ± 1.7 | 1.3 ± 0.11 | 350 ± 32* | 79 ± 1.1* | 160 ± 40 | 130 ± 33* | −3.4 |

*P ≦ 0.050 compared to untreated NL4-3-infected mice (group E) by Mann-Whitney U test.
^{†}group E: mouse #29 died Nov. 8, 2006 (cause unknown).

Treatment with one dose of 200 mg/kg albumin-conjugated Compound VIII), (fusion inhibitor concentration) reduced implant viral RNA by 3.3 log₁₀ and p24 by >95% compared to untreated infected mice. Four of six treated mice had no detectable p24 and one of six mice had no detectable p24 or HIV-1 RNA. A single dose of albumin-conjugated Compound VIII in these mice also protected immature and mature T-cells from virus-mediated cytopathicity and depletion and reduction in the CD4/CD8 ratio. In stark contrast to our findings with the albumin-conjugated Compound VIII, a single pre-exposure dose of Truvada (200 mg/kg tenofovir DF and 133 mg/kg emtricitabine) 24 hours before virus inoculation reduced viral RNA by only 0.8 log10 and p24 by 59% and was substantially less effective in preventing thymocyte depletion. It was observed that similarly potent activity of albumin-conjugated Compound VIII in SCID-hu mice treated once daily with 20 mg/kg beginning either 1 or up to 5 days after virus inoculation. In other SCID-hu studies, albumin-conjugated Compound VIII was 30-100 times more potent that C34 when given twice-daily beginning 1 day before inoculation.

This confirms the potent in vivo anti-HIV activity of albumin-conjugated Compound VIII. The potent activity observed for a single pre-exposure dose supports further preclinical and clinical development of this long-acting fusion inhibitor and confirms the usefulness of the SCID-hu Thy/Liv model for evaluation of in vivo antiretroviral efficacy.

### Example 2

### Evaluation of the Antiviral Activity of Albumin-Conjugated Compound Viii with Decreasing Dosing Frequency and Delayed Dosing Against HIV-1 NL4-3 in SCID-hu Thy/Liv Mice Treated by Subcutaneous Injection

TABLE 4
Protocol

| | |
|---|---|
| Implantation date | May 18, 2006 |
| Implant age | 18 weeks |
| Donor ID | #051706 |
| Inoculation date | Sep. 19, 2006 |
| Virus | HIV-1 NL4-3; batch lipo IV (diluted 1:3) |
| Inoculum | 1,000 TCID₅₀ per implant |
| Termination date | Oct. 10, 2006 (21 days after inoculation) |
| Drugs | albumin-conjugated Compound VIII), (Conjuchem, |
| | Montreal, lots #7/13/06 and #9/28/06) |
| Vehicle | N/A |
| Route | subcutaneous |
| Dosing | twice daily, or every fourth day, or every eighth |
| | day for 22 days |
| Volume | 200 μl per dose (400 μl for |
| | once-daily dosing) |
| Treatment initiation | 1 day before, 1 day, or 5 days after virus inoculation |

TABLE 5
Results

| | | | | | | | | HIV-1 | | |
|---|---|---|---|---|---|---|---|---|---|---|
| | | | | | | | | RNA | | |
| | | | | | | | | log₁₀ | FACS analysis | |
| | | | | | | p24 | | copies/ | Gag-p24⁺ | |
| | Mice/ | | | Dose | Dosing | (pg/10⁶ | (% of | 10⁶ | thymocytes | CD4⁺CD8⁺ |
| Group | group | Virus | Drug | (mg/kg/day) | Frequency | cells) | control) | cells | (%) | (%) |
| A | 7 | NL4-3 | albumin- | 20 (300 | Q4D | 8.5 ± 4.8* | 1.6 ± 0.91 | | 1.8 ± 0.31* | 74 ± 8.5* |
| | | | Conjugated | total) | | | | | | |
| | | | Compound | | | | | | | |
| | | | VIII) | | | | | | | |
| B | 6 | NL4-3 | albumin- | 20 (300 | Q8D | 210 ± 51* | 39 ± 9.5 | | 2.2 ± 0.41* | 79 ± 1.3* |
| | | | Conjugated | total) | | | | | | |
| | | | Compound | | | | | | | |
| | | | VIII), | | | | | | | |
| C^{†} | 8 | NL4-3 | albumin- | 20 (300 | day −1, | 41 ± 23* | 7.7 ± 4.3 | | 1.1 ± 0.37* | 83 ± 1.4* |
| | | | Conjugated | total) | 0, | | | | | |
| | | | Compound | | BID | | | | | |
| | | | VIII), | | | | | | | |
| D | 6 | NL4-3 | albumin | 20 (300 | day | negative* | 0.0 ± 0.0 | | 0.46 ± 0.05* | 86 ± 0.65* |
| | | | Conjugated | total) | +1, | | | | | |
| | | | Compound | | BID | | | | | |
| | | | VIII), | | | | | | | |
| E | 7 | NL4-3 | albumin | 20 (300 | day | negative* | 0.0 ± 0.0 | | 0.51 ± 0.15* | 76 ± 5.5* |
| | | | Conjugated | total) | +5, | | | | | |
| | | | Compound | | BID | | | | | |
| | | | VIII), | | | | | | | |
| F | 7 | NL4-3 | — | untreated | — | 530 ± 87 | 100 ± 16 | | 5.1 ± 0.66 | 36 ± 9.4 |
| G | 5 | medium | — | untreated | — | negative | 0.0 ± 0.0 | | 0.32 ± 0.05* | 86 ± 1.1* |

| | FACS analysis | | | | | Total | Live | Body |
|---|---|---|---|---|---|---|---|---|
| | | | | W6/32 mean | Live | cell | thymocyte | weight |
| | CD4⁺ | CD8⁺ | CD4/CD8 | fluorescence | hymocytes | yield | yield | change |
| Group | (%) | (%) | ratio | intensity | (%) | (10⁶) | (10⁶) | (%) |
| A | 8.9 ± 2.8* | 9.4 ± 4.6* | 1.2 ± 0.11 | 690 ± 49* | 70 ± 5.7* | 84 ± 23* | 63 ± 19* | +4.1 |
| B | 7.5 ± 0.63* | 6.0 ± 0.52* | 1.2 ± 0.04 | 1600 ± 230* | 77 ± 1.6* | 150 ± 20* | 110 ± 14* | +0.12 |
| C^{†} | 6.0 ± 0.57* | 4.8 ± 0.52* | 1.3 ± 0.02 | 1000 ± 120* | 79 ± 1.4* | 120 ± 17* | 91 ± 14* | +0.93 |
| D | 5.2 ± 0.27* | 3.9 ± 0.16* | 1.4 ± 0.02 | 620 ± 32* | 76 ± 1.6* | 140 ± 21* | 110 ± 17* | −1.0* |
| E | 6.0 ± 0.32* | 8.7 ± 3.9* | 1.1 ± 0.15 | 710 ± 38* | 70 ± 7.4* | 75 ± 21 | 56 ± 15* | +1.1 |
| F | 19 ± 2.2 | 24 ± 4.1 | 0.82 ± 0.06 | 3000 ± 550 | 52 ± 5.7 | 27 ± 5.3 | 16 ± 4.2 | +2.4 |
| G | 4.8 ± 0.49* | 37 ± 0.46* | 1.3 ± 0.06 | 900 ± 78* | 77 ± 0.88* | 120 ± 33* | 90 ± 25* | +4.1 |

*P ≦ 0.050 compared to untreated NL4-3-infected mice (group F) by Mann-Whitney U test.
^{†}group C: mouse #19 died Sep. 28, 2006 (cause unknown).

### Example 3

### Example 3A

### Experimental Procedures

The following procedures were used throughout the experiments performed to obtain the results discussed in detail below.

Peptide Synthesis

Synthesis of the CHR peptide analogs were performed using an automated solid-phase procedure on a Symphony Peptide Synthesizer with manual intervention during the generation of the peptides. The synthesis was performed on Fmoc-protected Ramage amide linker resin, using Fmoc-protected amino acids. Coupling was achieved by using O-benzotriazol-1-yl-N,N,N′,N′-tetramethyl-uronium hexafluorophosphate (HBTU) and diisopropylethylamine (DIEA) as the activator cocktail in N,N-dimethylformamide (DMF) solution. The Fmoc protective group was removed using 20% piperidine/DMF. A Boc-protected amino acid was used at the N-terminus in order to generate the free α-N-terminus following cleavage of the peptides from the resin. Sigmacoated glass reaction vessels were used during the synthesis.

When the maleimido is positioned at the C-terminus portion of the molecule (Table 6, albumin-conjugated Compound VII and acetylated-conjugated Compound X), the solid-phase synthesis of the peptide was initiated by the addition of Fmoc-Lys(Aloc). Aloc is a specific orthogonal protective group stable to acidic medium. The peptide chain was then elongated on solid support via the sequential addition of amino acids having their side chains protected with groups labile to acidic medium. When the peptide chain was completed, the Aloc protective group on the C-terminal lysine was removed selectively using tetrakistriphenylphosphine Palladium. The Fmoc-aminoethoxy ethoxy acetic acid (AEEA) linker was then chemically coupled to the unprotected lysine. Following classical Fmoc deprotection protocols, maleimide proprionic acid (MPA) was then chemically coupled to the AEEA spacer. Finally, the acid labile protecting groups were removed from the peptide and the peptide was then cleaved from the solid support using a strong acidic cocktail. When the maleimido is positioned at the N-terminus portion of the molecule (Table 6, maleimido-Compound VIII, albumin-conjugated Compound VIII), and albumin-conjugated-MPA-AEEA-Compound VIII, the solid-phase synthesis of the peptide was initiated by the native amino-acid sequence of the fusion peptide inhibitor.

TABLE 6

| | |
|---|---|
| HSA^{a} | Human Serum Albumin |

| | |
|---|---|
| C34 | (628)WMEWDREINNYTSLIHSLIEESQNQQEKNEQELL |
| | (661)-CONH₂^{b} |
| maleimido- | MPA^{c}-AEEA^{d}-(628)WMEWDREINNYTSLIHSLIEESQ |
| Compound | NQQEKNEQELL(661)-CONH₂^{b} |
| VIII | |
| albumin- | [HSA^{a}-CYS34^{e}]-MPA^{c}-AEEA^{d}-(628)WMEWDREIN |
| conjugated | NYTSLIHSLIEESQNQQEKNEQELL(661)-CONH₂^{b} |
| Compound | |
| VIII | |
| albumin- | [HSA^{a}-Cys34^{e}]-MPA-(628)WMEWDREINNYTSLIH |
| conjugated | SLIEESQNQQEKNEQELL(661)-CONH₂^{b} |
| Compound | |
| VII | |
| albumin- | (628)WMEWDREINNYTSLIHSLIEESQNQQEKNEQELL |
| conjugated | (661)K(εN)-AEEA^{d}-MPA^{c}-[CYS34^{e}-HSA^{a]} |
| Compound | |
| VI | |
| T-20 | Ac^{f}-(638)YTSLIHSLIEESQNQQEKNEQELLELDKWA |
| | SLWNWF(673)-CONH₂^{b} |
| albumin- | [HSA^{a}-CYS34^{e}]-MPA^{c}-AEEA^{d}-(638)YTSLIHSLI |
| conjugated | EESQNQQEKNEQELLELDKWASLWNWF(673)-CONH₂^{b} |
| Compound | |
| IX | |
| albumin- | Ac^{f}-(638)YTSLIHSLIEESQNQQEKNEQELLELDKWA |
| conjugated | SLWNWF(673)K(εN)-AEEA^{d}-MPA^{c}-[CyS34^{e}- |
| Compound | HSA^{a}] |
| X | |

^{a}HSA, human serum albumin
^{b}CONH₂, carboxamide
^{c}MPA, maleimide proprionic acid
^{d}AEEA, amino ethyl ethoxy acetic acid
^{e}CyS34 cysteine-34 of albumin
^{f}Ac, acetyl

Peptide Purification

Each product was purified by preparative reverse—phase HPLC, using a Varian (Dynamax) preparative binary HPLC system. Purification of all DAC peptides were performed using a Phenomenex Luna phenyl-hexyl (10 micron, 50 mm×250 mm) column equilibrated with a water/TFA mixture (0.1% TFA in H₂O; Solvent A) and acetonitrile/TFA (0.1% TFA in CH₃CN; Solvent B). Elution was achieved at 50 mL/min by running various gradients of Solvent B over 180 min. Fractions containing peptide were detected by UV absorbance (Varian Dynamax UVD II) at 214 and 254 nm.

Fractions were collected in 25 mL aliquots. Fractions containing the desired product were identified by mass after direct injection onto LC/MS. The selected fractions were subsequently analyzed by analytical HPLC (20-60% B over 20 min; Phenomenex Luna 5 micron phenyl-hexyl, 10 mm×250 mm column, 0.5 mL/min) to identify fractions with >90% purity for pooling. The pool was then freeze-dried using liquid nitrogen and subsequently lyophilized for at least 2 days yielding a white powder.

Preparation of Albumin Conjugates

The conjugation of maleimido-C34 and maleimido-T-20 derivatives to cysteine-34 of HSA and subsequent purification using hydrophobic interaction chromatography has recently become an efficient process. The conjugation step involves mixing each maleimido-peptide with a 25% solution of HSA (Cortex-Biochem, San Leandro, Calif.) and incubating for 30 min at 37° C. Using an ÄKTA purifier (GE Healthcare), the resulting mixtures were loaded at a flow rate of 2.5 ml/min directly onto a 50 ml column packed with butyl sepharose 4 fast flow resin (GE Healthcare) equilibrated in 20 mM sodium phosphate buffer (pH 7) composed of 5 mM sodium octanoate and 750 mM (NH₄)₂SO₄. Under these conditions, the C34-HSA conjugates adsorbed onto the hydrophobic resin whereas essentially all non-conjugated HSA eluted within the void volume of the column. Each conjugate was further purified from any free (unreacted) maleimido-C34 derivative by applying a linear gradient of decreasing (NH₄)₂SO₄ concentration (750-0 mM) over four column volumes. Each purified conjugate was then desalted and concentrated in water using 10 kDa ultracentrifugal filter devices (Amicon; Millipore, Bedford, Mass.). Finally, each conjugate solution was reformulated in an isotonic buffer solution at pH 7. Mass spectrometry of each purified sample confirmed the most abundant protein product corresponded to a 1:1 covalent complex of HSA with each maleimido derivative, and reverse-phase HPLC analysis of each purified sample confirmed the removal of essentially all unbound (free) maleimido derivative. Each albumin conjugate was formulated using sterile 0.9% NaCl and T-20 (obtained from the San Francisco General Hospital pharmacy) was dissolved in sterile water for injection and adjusted to pH 7 with HCl.

Anti-HIV Efficacy Evaluation in Fresh Human PBMCs

HIV-1 IIIB was obtained through the AIDS Research and Reference Reagent Program, Division of AIDS, NIAID, NIH courtesy of Dr. Robert C. Gallo (Popovic M E, Read-Connole E, Gallo R C (1984) T4 positive human neoplastic cell lines susceptible to and permissive for HTLV-III. Lancet ii: 1472-1473; Popovic M, Sarngadharan M G, Read E, Gallo R C (1984) Detection, isolation, and continuous production of cytopathic retroviruses (HTLV-III) from patients with AIDS and pre-AIDS. Science 224:497-500; Ratner L et al. (1985) Complete nucleotide sequence of the AIDS virus, HTLV-III. Nature 313:277-283). Fresh human peripheral blood mononuclear cells (PBMCs), seronegative for HIV and HBV, were isolated from blood of screened donors (Biological Specialty Corporation; Colmar, Pa.) using Lymphocyte Separation Medium (LSM; Cellgro® by Mediatech, Inc.; density 1.078+/−0.002 g/ml) following the manufacturer's instructions. Cells were stimulated by incubation in 4 μg/mL Phytohemagglutinin (PHA; Sigma) for 48-72 hours. Mitogenic stimulation was maintained by the addition of 20 U/mL recombinant human IL-2 (R&D Systems, Inc) to the culture medium. PHA-stimulated PBMCs from at least two donors were pooled, diluted in fresh medium and added to 96-well plates at 5×10⁴ cells/well. Cells were infected (final MOI≈0.1) in the presence of 9 different concentrations of test compounds (triplicate wells/concentration) and incubated for 7 days. To determine the level of virus inhibition, cell-free supernatant samples were collected for analysis of reverse transcriptase activity (Buckheit R W, Swanstrom R (1991) Characterization of an HIV-1 isolate displaying an apparent absence of virion-associated reverse transcriptase activity. AIDS Res Hum Retrovir 7:295-302). Following removal of supernatant samples, compound cytotoxicity was measured by the addition of 3-(4,5-dimethylthiazol-2-yl)-5-(3-carboxymethoxyphenyl)-2-(4-sulfophenyl)-2H-tetrazolium (MTS; CellTiter 96 Reagent, Promega) following the manufacturer's instructions. Using an in-house computer program, IC₅₀ (50%, inhibition of virus replication), IC₉₀ (90%, inhibition of virus replication), TC₅₀ (50% reduction in cell viability) and selectivity index (IC₅₀/TC₅₀) were determined. AZT (nucleoside reverse transcriptase inhibitor) was used as the assay control compound.

Viruses

The following reagent was obtained through the AIDS Research and Reference Reagent Program, Division of AIDS, NIAID, NIH: pNL4-3 from Malcolm Martin. (Adachi A et al. (1986) Production of acquired immunodeficiency syndrome-associated retrovirus in human and nonhuman cells transfected with an infectious molecular clone. J Virol 59:284-291.)

NL4-3 from the AIDS Reagent Program contains an unexpected variant DIV (G36D) mutation in gp41, which confers 8-fold resistance to T-20 in vitro A T-20-sensitive NL4-3 (NL4-3G) was altered by site-directed mutagenesis to match the consensus sequence at amino acid position 36 (aspartic acid replaced by glycine) of gp41. Stocks of NL4-3G and NL4-3D (original clone) were prepared by transfection of 293T cells and collection of supernatants on days 3. Virus stocks were titrated by 50% endpoint assay in PHA-activated PBMCs with p24 detection by ELISA.

SCID-hu Thy/Liv Mice

Human fetal thymus and liver were obtained through services provided by a nonprofit organization (Advanced Bioscience Resources) in accordance with federal, state, and local regulations. Complantation of thymus and liver fragments under the kidney capsule to create SCID-hu Thy/Liv mice and inoculation of the Thy/Liv implants with HIV-1 was carried out as described (Stoddart C A et al. (2007) Validation of the SCID-hu Thy/Liv mouse model with four classes of licensed antiretrovirals. PLoS ONE 2:e655; Greenberg M L, Cammack N (2004) Resistance to enfuvirtide, the first HIV fusion inhibitor. J Antimicrob Chemother 54:333-340.). Male C.B-17 SCID (model #CB17SC-M, homozygous, C.B-Igh-1^{b}/IcrTac-Prkdc^{scid}) mice were obtained at 6-8 weeks of age from Taconic, and cohorts of 50-60 SCID-hu Thy/Liv mice each were implanted with tissues from a single donor. Drugs were administered to groups of SCID-hu Thy/Liv mice (n=5-7) by subcutaneous injection at the indicated dosage levels (peptide alone excluding albumin “albumin-conjugated Compound VIII”) beginning 24 h before direct injection of 1,000 TCID₅₀ (in 50 μl) HIV-1 NL4-3G or NL4-3D or RPMI 1640 (mock infection) into each Thy/Liv implant. Implants were inoculated 18 weeks after tissue implantation and were collected 21 days after virus inoculation. The Thy/Liv implants were collected from euthanized mice, and single-cell suspensions were prepared by dispersing the implant through nylon mesh and processed for p24 ELISA, bDNA assay, and FACS analysis as described (Buckheit R W, Swanstrom R (1991) Characterization of an HIV-1 isolate displaying an apparent absence of virion-associated reverse transcriptase activity. AIDS Res Hum Retrovir 7:295-302; Stoddart C A et al. (2007) Validation of the SCID-hu Thy/Liv mouse model with four classes of licensed antiretrovirals. PLoS ONE 2:e655; Greenberg M L, Cammack N (2004) Resistance to enfuvirtide, the first HIV fusion inhibitor. J Antimicrob Chemother 54:333-340.). Animal protocols were approved by the UCSF Institutional Animal Care and Use Committee.

Flow Cytometry

Implant cells were stained with phycoerythrin cyanine dye CY7-conjugated anti-CD4 (BD Biosciences), phycoerythrin cyanine CY5.5-conjugated anti-CD8 (Caltag), allophycocyanin cyanine CY7-conjugated anti-CD3 (eBiosciences), and phycoerythrin-conjugated anti-W6/32 (DakoCytomation). Cells were fixed and permeabilized with 1.2% paraformaldehyde and 0.5% Tween 20, stained with fluorescein isothiocyanate-conjugated anti-p24 (Beckman Coulter), and analyzed on an LSR II (BD Biosciences). After collecting 100,000 total cell events, percentages of marker-positive (CD4⁺, CD8⁺, and CD4⁺ CD8⁺) thymocytes in the implant samples were determined by first gating on a live lymphoid cell population identified by forward- and side-scatter characteristics and then by CD3 expression.

### Example 3B

### Results

Antiviral Activities in-vitro Using PBMC Based Assays

The antiviral activity of each albumin conjugate was compared to the original peptide inhibitors in vitro using a PBMC-based assay against HIV-1 IIIB (Popovic M E, et al. (1984) Lancet ii: 1472-1473; Popovic M, et al. (1984) Science 224:497-500; Ratner L et al. (1985) Nature 313:277-283; Buckheit R W, Swanstrom R (1991) AIDS Res Hum Retrovir 7:295-302.). Interestingly, the antiviral activity of albumin-conjugated Compound VIII, albumin-conjugated Compound VII, and albumin-conjugated Compound VI were all found to be essentially equipotent to C34 peptide and T-20 in vitro. That is, placement of the reactive maleimide group at either the N-terminus (albumin-conjugated Compound VIII and albumin-conjugated Compound VII) or C-terminus (albumin-conjugated Compound VI) of the C34 peptide followed by albumin conjugation did not alter the antiviral activity of the fusion inhibitor (Table 7). Following albumin conjugation to T-20, there was excellent retention of antiviral activity when the reactive peptide is designed such that conjugation occurs at the N-terminal end of the peptide (albumin-conjugated Compound III), whereas a significant decrease in the antiviral activity for this peptide was observed when conjugation to albumin occurs at the C-terminal end of the peptide (Compound X).

TABLE 7

| | IC₅₀ | | Selectivity |
|---|---|---|---|
| Compound | (nM) | IC₉₀ (nM) | Index |
| HAS | NA | NA | NA |
| C34 | 0.6 | 2.8 | >255 |
| maleimido- | NP | NP | NP |
| Compound VIII | | | |
| albumin-conjugated | 1.8 | 13.5 | >81.5 |
| Compound VIII | | | |
| albumin-conjugated | 11.2 | 30.2 | >22.4 |
| Compound VII | | | |
| T20 | 2.2 | 9.5 | >109 |
| albumin-conjugated | 10.7 | 31.7 | >23.4 |
| Compound IX | | | |
| albumin-conjugated | 87.0 | >2,000 | >23.0 |
| Compound X | | | |
| AZT | 2.9 | 26.9 | >346 |

NA = IC₅₀ not achieved
NP = not performed

### Example 3C

### Antiviral Activity in SCID-hu Thy/Liv Mice

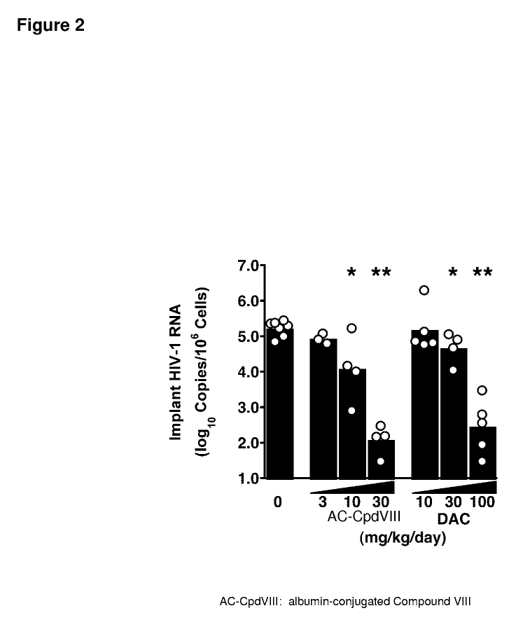

The human thymus implant in SCID-hu Thy/Liv mice supports long-term differentiation of human T cells and has been standardized and validated for the preclinical evaluation of antiviral compounds against HIV-1 (Stoddart C A et al. (2007) PLoS ONE 2:e655.). When albumin-conjugated Compound VIII was compared head-to-head to the unconjugated peptide, maleimido-Compound VIII, in SCID-hu Thy/Liv mice treated twice daily beginning 24 h before inoculation with NL4-3G, preformed conjugate-Compound VIII was approximately three times more potent than maleimido-Compound VIII in reducing HIV-1 RNA in the implants (FIG. 2). Specifically, viral RNA was reduced by 3.1 log₁₀ with 30 mg/kg per day albumin-conjugated Compound VIII and by 2.7 log₁₀ with 100 mg/kg/day maleimido-Compound VIII.

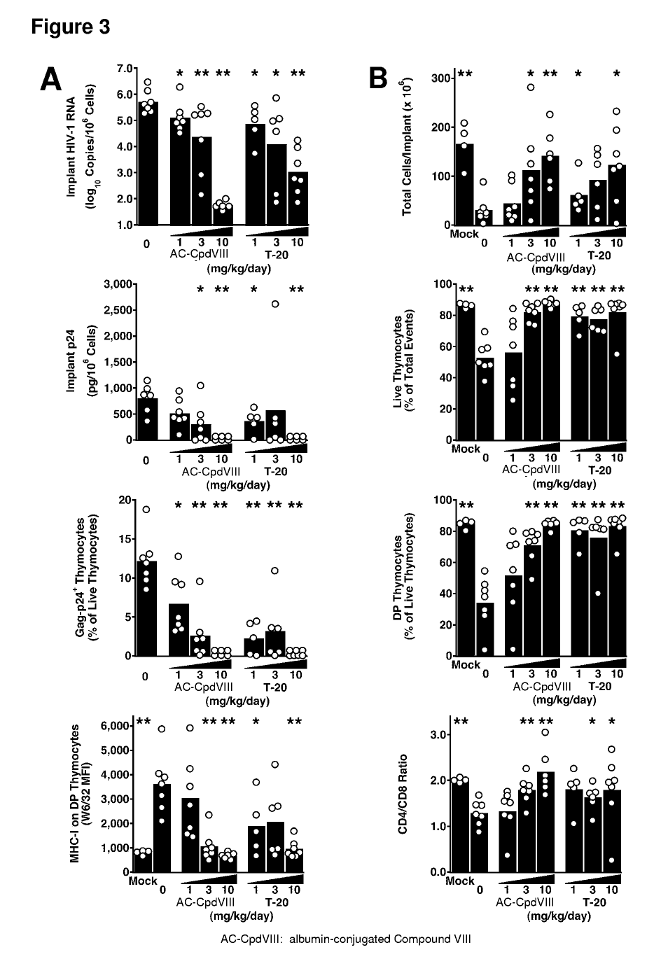

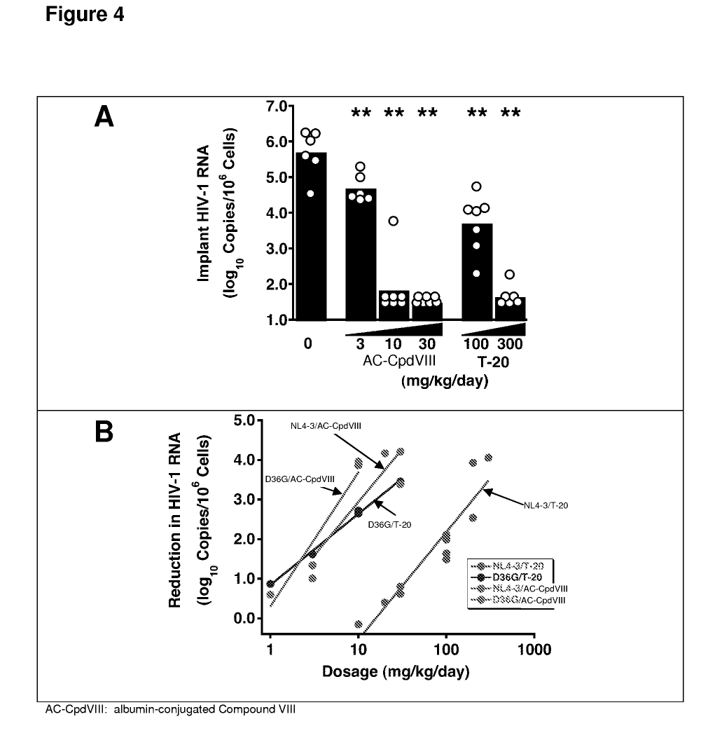

When albumin-conjugated Compound VIII and T-20 were compared in SCID-hu Thy/Liv mice treated twice daily beginning 24 h before inoculation with NL4-3G, the two drugs had comparable dose-dependent activity at 1, 3, and 10 mg/kg per day. Treated mice had statistically significant reductions in viral RNA, p24, Gag-p24⁺ thymocytes, and IFN-α-induced MHC-I expression on DP thymocytes compared to untreated mice (FIG. 3A). Reductions in viral load were accompanied by virtually complete protection of the Thy/Liv implants from thymocyte depletion, in terms of total cellularity, thymocyte viability, percentage of immature cortical CD4⁺ CD8⁺ (double-positive, DP) thymocytes, and CD4/CD8 ratio (FIG. 3B). In a third SCID-hu study to compare the activity of albumin-conjugated Compound VIII and T-20 against T-20-resistant NL4-3D, 10 mg/kg per day of albumin-conjugated Compound VIII reduced viral RNA by 2 log₁₀ more than did T-20 at 100 mg/kg per day (FIG. 4A). When the data for viral RNA reductions in seven independent SCID-hu mouse experiments are analyzed together, it is clear that antiviral potency against T-20-resistant NL4-3D was maintained for albumin-conjugated Compound VIII (FIG. 4B).

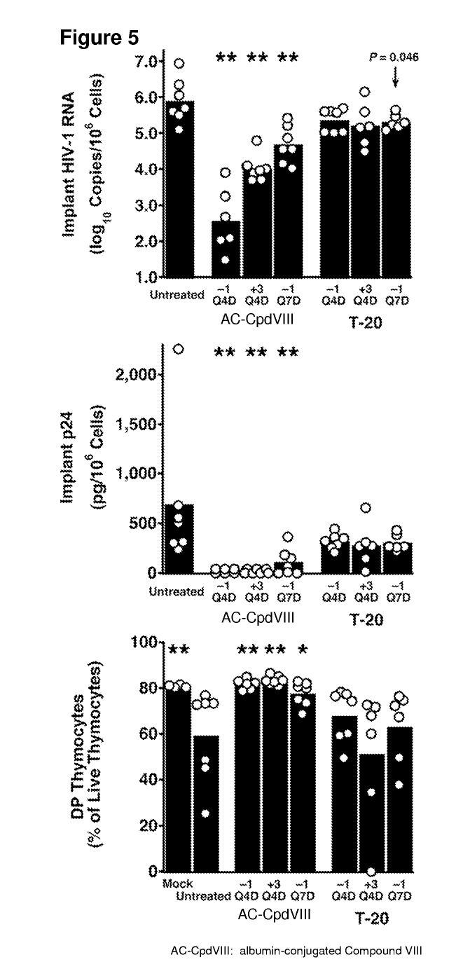

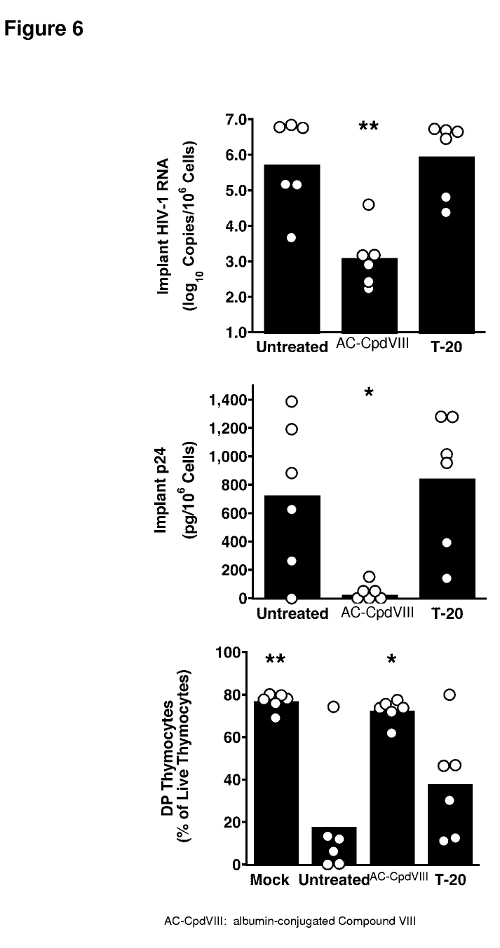

Finally, to determine whether the superior pharmacokinetics of preformed conjugate-Compound VIII would require less frequent dosing compared to T-20, we compared the activities of each drug following dosing every 4^{th} day as opposed to twice daily, following +1, +3, and +5 days post-infection (FIG. 5). Contrary to T-20, there was excellent conservation of antiviral activity for albumin-conjugated Compound VIII when the initiation of dosing was delayed. With the Q4D dosing regimen, T-20 caused no reduction in HIV-1 RNA or p24 nor did it protect from thymocyte depletion. Finally, following a single elevated preexposure dose of each drug 24 h before NL4-3G inoculation (FIG. 6), only albumin-conjugated Compound VIII was highly effective at reducing viral RNA by nearly 3 log₁₀ and p24 to almost undetectable levels.

### Example 3D

### Discussion

Synthetic peptides based upon the N-terminal helical region (NHR) and the C-terminal helical region (CHR) sequences of HIV gp41 have been shown to inhibit HIV entry by competing for exposed gp41 binding sites during the multi-step fusion process (Chan D C, et al. (1997) Cell 89: 263-273; Chan D C, et al (1998) Proc Natl Acad Sci USA 95: 15613-15617.). In the clinic, the most successful of these peptides is T-20 (Fuzeon® from Trimeris/Roche Applied Sciences) derived from the CHR of gp41. As compared to small molecules, the commercial utility of peptides is often limited by their high cost as well as their short half-lives and poor distribution in vivo. We sought to address these shortcomings by engineering CHR peptides (C34 and T-20) to bond covalently to cysteine-34 of human albumin as has already been done for other classes of peptides (Holmes D L et al. (2000) Bioconj Chem 11: 439-444; Leger R et al. (2003) Bioorg & Med Chem Lett 13: 3571-3575; Leger R et al. (2004) Bioorg & Med Chem Lett 14: 841-845; Leger R et al. (2004) Bioorg & Med Chem Lett 14: 4395-4398; Jette L et al. (2005) Endocrinology 146: 3052-3058; Thibaudeau K et al. (2005) Bioconj Chem 16: 1000-1008.). That is, we postulated the CHR-peptide-HSA conjugates would experience a half-life in the body closer to that of albumin as opposed to a much shorter half-life for the original fusion inhibitor.

Our results show that NHR of gp41 is more accessible than what had been originally believed. For example, to allow for such competitive inhibition to take place as that shown using albumin-conjugated Compound VIII, gp41 may be involved in a conformational equilibrium exposing the NHR region in the absence of target cells (e.g. in the context of a cell-free virus or infected cell), or that the pre-hairpin intermediate formed within the “entry claw” (Sougrat R et al. (2007) PLoS Pathogens 3: 0571-0581.), is sufficiently solvent-exposed prior to the formation of the six helix bundle and subsequent lipid mixing and membrane puncturing steps. That is, with a Mw of ˜71 kDa for albumin-conjugated Compound VIII, our results suggest the molecular weight cutoff for accessing the NHR-trimer of gp41 is much greater than previously reported, e.g. <25 kDa (Hamburger A E, et al. (2005) J Biol Chem 280: 12567-12572.). Second, the N-terminal segment of the C34 peptide, ⁶²⁸WMEW⁶³¹, represents the gp41 coiled-coil cavity binding residues postulated to be essential for C34 peptide's ability to inhibit HIV-1 entry (Chan D C, et al. (1997) Cell 89: 263-273; Chan D C, et al. (1998) Proc Natl Acad Sci USA 95: 15613-15617.). Therefore, in the case of either albumin-conjugated Compound VIII (composed of AEEA linker) or albumin-conjugated-Compound VII (absence of AEEA linker), how is it possible for the 628WMEW631 segment of C34 peptide to reach the NHR of gp41, and simultaneously, be permanently bonded and positioned in close proximity to the surface of albumin? One possible explanation for the retention of antiviral activity for albumin-conjugated Compound VIII and albumin-conjugated Compound VII is the fact that serum albumin is a highly flexible protein capable of being induced to adopt several conformational states (Peters T, Jr (1996) All about albumin-biochemistry, genetics, and medical applications, Copyright by Academic Press, Inc.). For example, since C34 peptide is permanently attached to cysteine-34 of albumin, it is possible local conformational rearrangements within the unconstrained N-terminal domain of albumin (e.g. absence of disulfide bridges) cause partial unwinding so as to facilitate correct insertion of the fusion inhibitor onto the NHR region of gp41. Therefore, it is not known whether positioning of C34 peptide elsewhere within the albumin molecule other than on cysteine-34 will lead to similar conservation of antiviral activity for this fusion inhibitor (e.g. lysine residues, N-terminal or C-terminal ends), or whether similar conservation of antiviral activity would be observed following permanent conjugation of C34 peptide to other abundant serum proteins of higher molecular weight such as transferrin or IgG. Hence, it is also possible the albumin molecule plays an active participatory role rather than merely serving as a protein cargo. For example, maleylated-, aconitylated-, and succinylated-albumin function as potent HIV-1 entry inhibitors in vitro (Takami M, et al. (1992) Biochim Biophys Acta 1180: 180-186; Jansen R W, et al. (1993) Mol Pharmacol 44: 1003-1007; Swart P J et al. (1996) J Drug Target 4: 109-116; Groenink M et al. (1997) AIDS Res Hum Retrovir 13: 179-185.).

Additionally, given that 24 out of the 34 amino-acid residues found in the C34 peptide overlaps with those found in T-20, how is it possible for T-20 to be a poorer candidate for albumin conjugation following modification at the C-terminus of this peptide whereas an improved retention of antiviral activity is observed when T-20 is modified at its N-terminus? One possible explanation for this finding is the recent evidence suggesting the mechanism of HIV-1 inhibition due to T-20 is distinct from that of C34 peptide (Liu S et al. (2005) J Biol Chem 280:11259-11273; Muñoz-Barroso I, et al. (1998) J Cell Biol 140:315-23; Kliger Y et al. (2001) J Biol Chem 276:1391-1397.). For example, T-20 has also been shown to inhibit recruitment of gp41 to the plasma membrane and its subsequent oligomerization at a post-lipid mixing step, whereas C34 peptide was found to be incapable of exerting its inhibitory effect following formation of the six helix bundle (Liu S et al. (2005) J Biol Chem 280:11259-11273.). That is, it has been proposed that T-20 performs such inhibitory functions following its insertion into plasma membrane and that the hydrophobic C-terminal segment of T-20, ⁶⁶⁶WASLWNWF⁶⁷³, was deemed critical for effectuating these hydrophobic interactions (Muñoz-Barroso I, et al. (1998) J Cell Biol 140:315-23; Kliger Y et al. (2001) J Biol Chem 276:1391-1397.). More specifically, T-20 inhibits gp41 recruitment and oligomerization by binding to the corresponding sequence within gp41 situated in close proximity to the plasma membrane (Muñoz-Barroso I, et al (1998) J Cell Biol 140: 315-23; Kliger Y et al. (2001) J Biol Chem 276:1391-1397.). Hence, the dramatic loss in antiviral activity observed for Compound X, where the ⁶⁶⁶WASLWNWF⁶⁷³ sequence is positioned directly adjacent to the albumin molecule, may be attributed to this peptide's inability to function at a post lipid-mixing step as efficiently as the unconjugated (free) T-20 peptide. Conversely, the ⁶⁶⁶WASLWNWF⁶⁷³ sequence less conformationally constrained in the design of compound IX. In summary, our results provide definitive supporting evidence for reports that have suggested that T-20 and C34 peptide do not function at the same steps of HIV-1 fusion.

The antiviral activity of albumin-conjugated Compound VIII was also assessed in vivo using the SCID-hu Thy/Liv mouse model and compared to that for maleimido-Compound VIII (FIG. 2) and T-20 (FIG. 3-6). By using an in vivo model, the advantages of dosing a fusion peptide inhibitor prefixed onto a carrier protein such as albumin become obvious. For example, we attribute the 3-fold increase in potency for albumin-conjugated Compound VIII over maleimido-Compound VIII to incomplete conjugation efficiency in vivo for the latter to mouse serum albumin following its subcutaneous administration to SCID-hu Thy/Liv mice. These antiviral results corroborate with pharmacokinetic profiles obtained from uninfected mice where only 40% exposure of C34 peptide was observed for maleimido-1505 as compared to that for albumin-conjugated Compound VIII following subcutaneous dosing of the two compounds (data not shown). Therefore, despite the recent observation in vitro that maleimido analogs of C34 peptide with structures similar to that of maleimido-1505 are also capable of forming a specific covalent bond to Lys⁵⁷⁴ located within the NHR of gp41 (Jacobs A et al. (2007) J Biol Chem 282: 32406-32413.), the reduced potency for albumin-conjugated Compound VIII may be accounted for by the fact that the unreacted (free) peptide is less stable against proteolytic enzymes and is subject to normal rapid clearance pathways.

The equipotent activity of albumin-conjugated Compound VIII and T-20 in vitro (Table 3) was corroborated using the T-20-sensitive NL4-3G clone in the in vivo model. However, the advantages of albumin-conjugated Compound VIII over T-20 become obvious when either the frequency of dosing is reduced from twice daily to every fourth day (FIG. 5) or when a single elevated preexposure dose is administered (FIG. 6). The ability of albumin-conjugated Compound VIII to outperform T-20 in vivo is due primarily to the significantly improved exposure and stability of the C34 peptide following albumin conjugation without abrogating the antiviral activity of the original peptide (Table 3). Finally, albumin-conjugated Compound VIII has also been shown to be highly active in vivo against the T-20-resistant NIA-3D (FIG. 4). Given that the amino-acid sequences of the C34 peptide and T-20 overlap and that Gly547 positioned within the NHR of gp41 is expected to bind near the C-terminal end of C34 peptide, the conserved antiviral activity of albumin-conjugated Compound VIII against NL4-3D provides definitive supporting evidence for the importance of the gp41 coiled-coil cavity binding residues, ⁶²⁸WMEW⁶³¹, which are absent in the structure of T-20 (Chan D C, et al. (1997) Cell 89: 263-273; Chan D C, et al (1998) Proc Natl Acad Sci USA 95: 15613-15617.). Taken together, these data confirm the highly potent in vivo anti-HIV activity of albumin conjugated-C34 peptide fusion inhibitor.

The results presented herein establish a proof-of-principle for this new class of albumin-peptide conjugates for inhibition of HIV or other viruses that have adopted similar mechanisms of membrane fusion and viral entry. As compared to unconjugated (free) peptide inhibitors, albumin conjugation may lead to a significantly improved exposure to the lymphatic system representing the anatomical home of approximately 98% of total HIV-infected cells (Stebbing J, et al. (2004) N Engl J Med 350:1872-1880). This improvement may be expected due primarily to significant steady-state lymph to plasma concentration ratios observed for serum albumin (Bent-Hansen L (1991) Acta Physiol Scand Suppl 603: 5-10 (Review); Porter C J H, Charman S A (2000) J Pharm Sci 89: 297-310.), and to the efficient lymphatic uptake, transport and permeability observed for subcutaneously injected proteins larger than 16-20 kDa (Porter C J H, Charman S A (2000) J Pharm Sci 89: 297-310.).

Finally, due to the high content of hydrophobic residues found in C34 peptide and many other antifusogenic peptides, albumin conjugation may also help remedy the low solubility limits commonly observed for this family of peptides when they are placed in simple aqueous formulations amenable for subcutaneous delivery (Otaka A M et al. (2002) Angew Chem Int Ed Engl 41: 2937-2940.). For example, the solubility limit of C34 peptide was found to be no more than 1 mg/ml in aqueous buffer whereas that of albumin-conjugated Compound VIII was found to be similar to that for albumin corresponding to approximately 16 mg/ml of C34 peptide (i.e. 25% (w/v) solution=250 mg/ml of albumin-conjugated Compound VIII≈16 mg/ml of C34 peptide).

In summary, conjugation of antifusogenic peptides through albumin's cysteine-34 overcomes the steric block commonly associated to the NHR trimer of gp41, and thus, offers hope for the discovery of novel, larger molecular weight molecules exhibiting potent and broadly neutralizing activity. One example of an albumin-conjugated C34 peptide HIV-1 fusion inhibitor, albumin-conjugated Compound VIII, may require less frequent dosing than T-20 and is likely to be an effective agent against T-20-resistant HIV-1 in humans. The potent activity we observed for a single elevated preexposure dose of albumin-conjugated Compound VIII supports further preclinical and clinical development of this promising antiviral approach and confirms the utility and flexibility of the SCID-hu Thy/Liv mouse model for the preclinical evaluation of in vivo antiretroviral efficacy and drug resistance.

Entry inhibitors of HIV-1 have been the focus of much recent research. C34, a potent fusion inhibitor derived from the HR2 region of gp41, was engineered into a 1:1 HSA conjugate through stable covalent attachment of a maleimido-C34 analog onto cysteine-34 of albumin. This bioconjugate, albumin-conjugated Compound VIII, was designed to require less frequent dosing and less peptide than T-20 and was assessed for its antifusogenic activity both in vitro and in vivo in the SCID-hu Thy/Liv mouse model. albumin-conjugated Compound VIII was essentially equipotent to the original C34 peptide and to T-20 in vitro. In HIV-1-infected SCID-hu Thy/Liv mice, T-20 lost activity with infrequent dosing whereas the antiviral potency of albumin-conjugated Compound VIII was sustained. The in vivo results are the direct result of significantly improved pharmacokinetic profile for the C34 peptide following albumin conjugation. Contrary to previous reports the gp41 NHR-trimer is poorly accessible to C34 fused to protein cargoes of increasing size (Hamburger A E, et al. (2005) J Biol Chem 280: 12567-12572), these results are the first demonstration of the capacity for a large, endogeneous serum protein to gain unobstructed access to the transient gp41 intermediates that exist during the HIV fusion process, and it supports further development of albumin conjugation as a promising approach to inhibit HIV-1 entry.

Entry of human immunodeficiency virus type 1 (HIV-1) into uninfected cells encompasses three main steps, the binding of gp120 to the CD4 receptor, the subsequent binding to coreceptor CXCR4 or CCR5, followed by the conformational changes of the ectodomain of HIV-1 gp41 critical to membrane fusion that ultimately permits the infection process. Several small molecule drug candidates, including those that inhibit binding to CD4 or to the CCR5 co-receptor, are either in human clinical trials or are close to market approval (Meanwell N A, Kadow J F (2003) Curr Opinion Drug Disc & Develop 6: 451-461; Olson W C, Maddon P J (2003) Curr Drug Targets-Infectious Disord 3: 283-294.). However, T-20 (DP-178, enfuvirtide, Fuzeon®, Trimeris/Roche Applied Sciences), a synthetic peptide based on the CHR sequence of HIV-1 gp41, remains the only compound marketed to date that targets the conformational rearrangements of gp41. It had been widely believed that T-20 inhibition was due to its ability to bind to the hydrophobic grooves of the NHR region of gp41 resulting in the inhibition of six-helix bundle formation (Kliger Y, Shai Y (2000 J Mol Biol 295: 163-168.). Contrary to this view, and despite the identification of less common escape mutants against T-20 with mutations found within the NHR of gp41 (Wei X et al. (2002) Antimicrob Agents Chemother 46: 1896-1905; Roman F D et al. (2003) J AIDS 33:134-139), recent studies suggest although T-20 is capable of targeting multiple sites in gp41 and gp120 (Liu S et al. (2005) J Biol Chem 280:11259-11273.). For example, T-20 binds and oligomerizes at the surface of membranes, thereby inhibiting recruitment and oligomerization of gp41 at the plasma membrane of infected cells and leading to the “clamping” of the fusion complex in the lipid-mixing intermediate for up to 6 h of co-culture of gp120-41-expressing cells with target cells (Muñoz-Barroso I, Durell S, Sakaguchi K, et al. (1998) J Cell Biol 140:315-23; Kliger Y et al. (2001) J Biol Chem 276:1391-1397.). Furthermore, it has also been shown the ectodomain of gp41 within a region immediately adjacent to the membrane-spanning domain having the peptide sequence, ⁶⁶⁶WASLWNWF⁶⁷³, constitutes a higher affinity site for T-20 than the NHR of gp41 (Muñoz-Barroso I, et al. (1998) J Cell Biol 140: 315-23; Kliger Y et al. (2001) J Biol Chem 276:1391-1397.). In contrast, another C-peptide, C34, composed of a peptide sequence which overlaps with T-20 but contains the gp41 coiled-coil cavity binding residues, ⁶²⁸WMEW⁶³¹, is known to compete with the CHR of gp41 for the hydrophobic grooves of the NHR region yet is incapable of functioning at a post-lipid mixing stage (Liu S et al. (2005) J Biol Chem 280:11259-11273.). Despite the successes of T-20, its commercial utility has been somewhat restricted to salvage therapy resulting from a) the need for twice-daily, subcutaneous dosing (90 mg of drug per dose) due to rapid excretion and metabolism associated with most peptide-based drugs, b) a high incidence of injection site reactions, c) a challenging manufacturing process due to its lengthy, specific amino-acid sequence, and d) a high cost to patients (Gilden D (1998) T-20 and Adefovir for salvage therapy—Expect no miracles. The Body—The Complete HIV/AIDS Resource; Manfredi R, Sabbatini S (2006) Curr Med Chem 13: 2369-2384.).

The challenge in developing therapeutic peptides is complicated primarily by their rapid renal clearance, poor distribution, and susceptibility to peptidase degradation. For a compound with antiviral activity, particularly peptide-based inhibitors such as T-20 targeting the HIV-1 fusion process, the ability to maintain a stable pharmacokinetic profile in humans is expected to confer several advantages. For example, such improvements may lead to better patient compliance due to a less frequent dosing regimen as well as lower cost of manufacturing and cost to patients. Despite recent predictions that cross-linking C-peptide inhibitors to larger proteins will likely reduce the potency of modified C-peptides (Hamburger A E, et al. (2005) J Biol Chem 280: 12567-12572.), we used albumin conjugation as a vehicle to achieve superior phamacokinetic profiles of fusion peptide inhibitors as has been performed using other classes of maleimido peptides such as dynorphin A (Holmes D L et al. (2000) Bioconj Chem 11: 439-444), natriuretic peptide (Léger R et al. (2003) Bioorg & Med Chem Lett 13: 3571-3575), Kringle 5 (Leger R et al. (2004) Bioorg & Med Chem Lett 14: 841-845), GLP-1 (Leger R et al. (2004) Bioorg & Med Chem Lett 14: 4395-4398), growth hormone-releasing factor (Jette L et al. (2005) Endocrinology 146: 3052-3058), and insulin (Thibaudeau K et al. (2005) Bioconj Chem 16: 1000-1008.). Such conjugation reactions may be performed in vivo by administering the compound directly into the human patient followed by conjugation to endogeneous albumin. Similarly, conjugation reactions may also be carried out in vitro by reacting the maleimido peptide with albumin prior to administering the bioconjugate to a subject. In this study, the C34 peptide derived from the CHR of gp41 (Chan D C, et al. (1997) Cell 89: 263-273; Chan D C, et al. (1998) Proc Natl Acad Sci USA 95: 15613-15617) was engineered into preformed albumin conjugates whereby specific covalent linkage to albumin was carried out through either the N-terminus or the C-terminus of the fusion inhibitor. Similarly, preformed albumin conjugates composed of maleimido-T-20 analogs were also generated. Each drug construct represented a 1:1 complex through specific and stable covalent attachment of the peptide to cysteine-34 of albumin, and each construct was assessed for its antiviral activity in vitro following infection in a peripheral blood mononuclear cell (PBMC)-based assay with the HIV-1 strain IIIB (Popovic M E, et al. (1984) Lancet ii:1472-1473; Popovic M, et al. (1984) Science 224:497-500; Ratner L et al. (1985) Nature 313:277-283; Buckheit R W, Swanstrom R (1991) AIDS Res Hum Retrovir 7:295-302.). Furthermore, using the SCID-hu Thy/Liv mouse model of HIV-1 infection (Stoddart C A et al. (2007). PLoS ONE 2:e655), we evaluated the antiviral activity of one C34-HSA conjugate, albumin-conjugated Compound VIII (FIG. 1), and found that while T-20 lost activity with infrequent dosing, the antiviral potency of albumin-conjugated Compound VIII was sustained.

1. A method of treating or preventing a virus selected from the group consisting of human immunodeficiency virus (HIV) infection, respiratory syncytial virus (RSV), human parainfluenza virus type 3 (HPIV-3), measles virus and simian immunodeficiency virus (SIV) in a subject, comprising
administering one or more initial doses of a modified antifusogenic peptide, or a conjugate thereof, to a subject, prior to infection or prior to the onset or recurrence of one or more symptoms associated with the infection, thereby treating or preventing the infection, wherein the modified antifusogenic peptide is a compound selected from the group consisting of (SEQ ID NOS 2, 49-51, 13, 13, 2 and 2, respectively, in order of appearance):

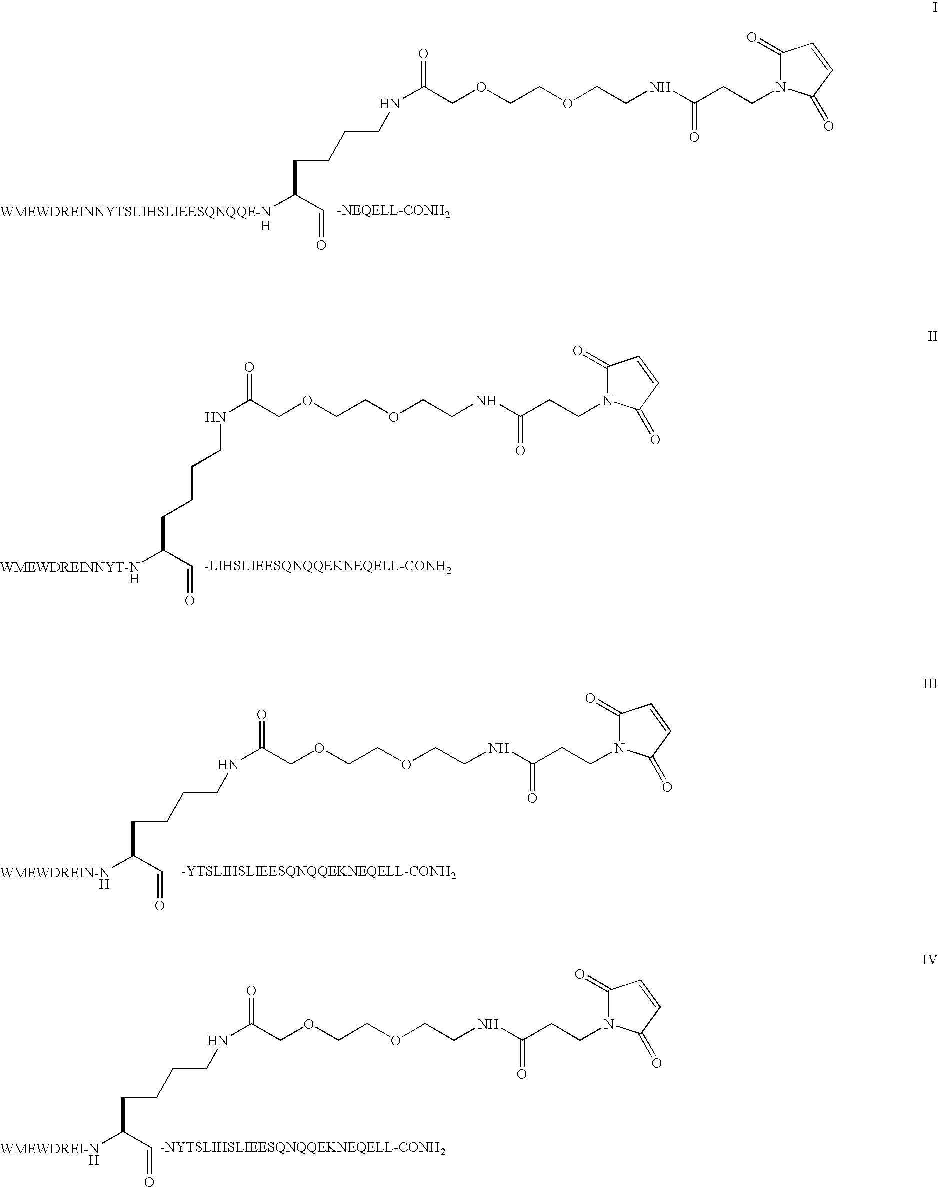

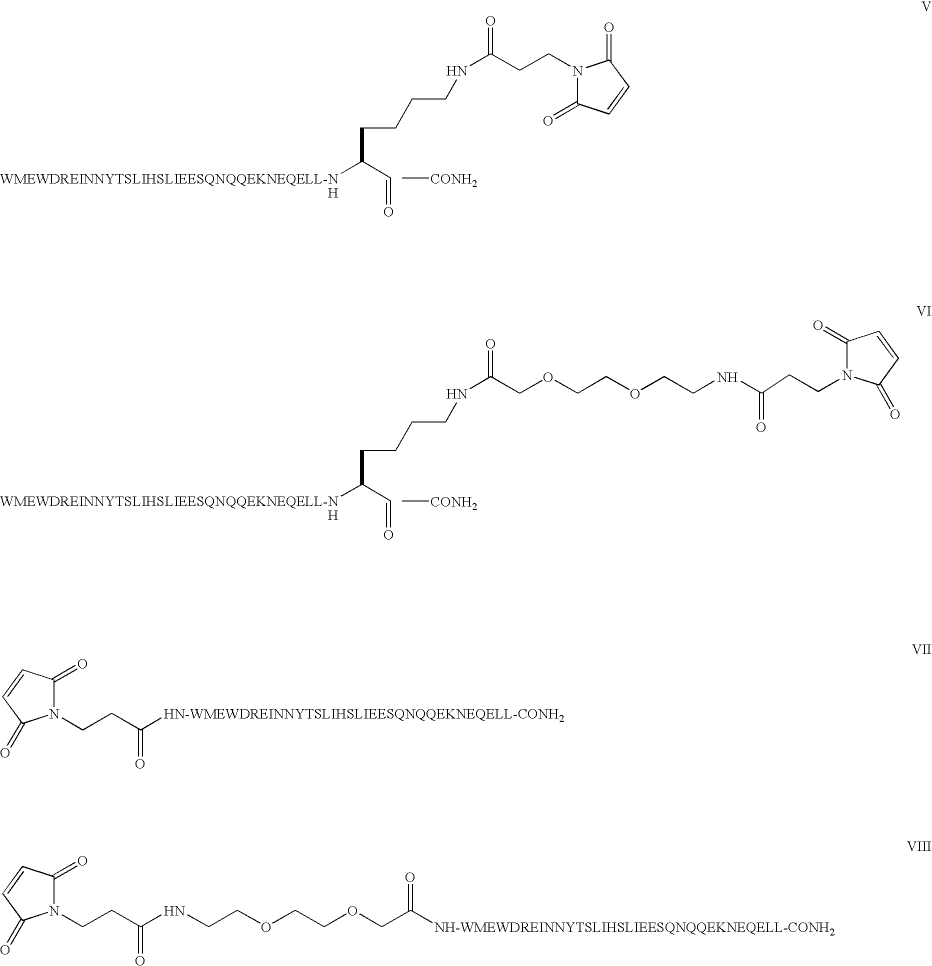

2. The method of claim 1, further comprising selecting the subject prior to infection or prior to the onset or recurrence of one or more symptoms associated with the infection.

3. The method of claim 1, wherein the one or more initial doses are in the range of about 30 mg/kg to about 75 mg/kg.

4. The method of claim 3, wherein the one or more initial doses are about 60 mg/kg.

5. The method of claim 1, wherein the one or more doses are in the range of about 150 mg/kg to about 300 mg/kg.

6. The method of claim 5, wherein the one or more doses are about 200 mg/kg.

7. The method of claim 1, wherein the one or more initial doses are of the modified antifusogenic peptide is administered at least twenty four hours prior to infection.

8. The method of claim 1, further comprising administering at least one subsequent dose of the modified antifusogenic peptide after administering the initial single dose.

9. The method of claim 8, wherein the at least one subsequent dose is administered three days after infection.

10. The method of claim 8, wherein the at least one subsequent dose is administered seven days after infection.

11. The method of claim 8, wherein the at least one subsequent dose is administered at time intervals selected from the group consisting of 1 hour, 2 hours, 4 hours, 8 hours, 12 hours, 24 hours, 4 days, 7 days, 14 days, 30 days, and 60 days.

12. The method of claim 11, further comprising administering the modified antifusogenic peptide at four-day intervals.

13. The method of claim 11, further comprising administering the modified antifusogenic peptide at seven-day intervals.

14. The method of claim 1, wherein the initial single dose is a single treatment interval comprising a dose of up to 200 mg/kg.

15. The method of claim 1, further comprising administering the modified antifusogenic peptide prophylactically.

16. The method of claim 1, wherein the method of administration is selected from the group consisting of subcutaneous, intraperitoneal, intramuscular, intravenously, and pulmonary inhalation.

17. The method of claim 1, wherein the modified antifusogenic peptide is covalently coupled to a blood protein.

18. The method of claim 17, wherein the blood protein is a recombinant protein.

19. The method of claim 17, wherein the blood protein is serum albumin or recombinant albumin.

20. The method of claim 19, wherein the modified antifusogenic peptide is covalently coupled to the Cys34 residue of albumin.

21. A dosage formulation comprising an antifusogenic peptide selected from the group consisting of (SEQ ID NOS 2, 49-51, 13, 13, 2 and 2, respectively, in order of appearance): 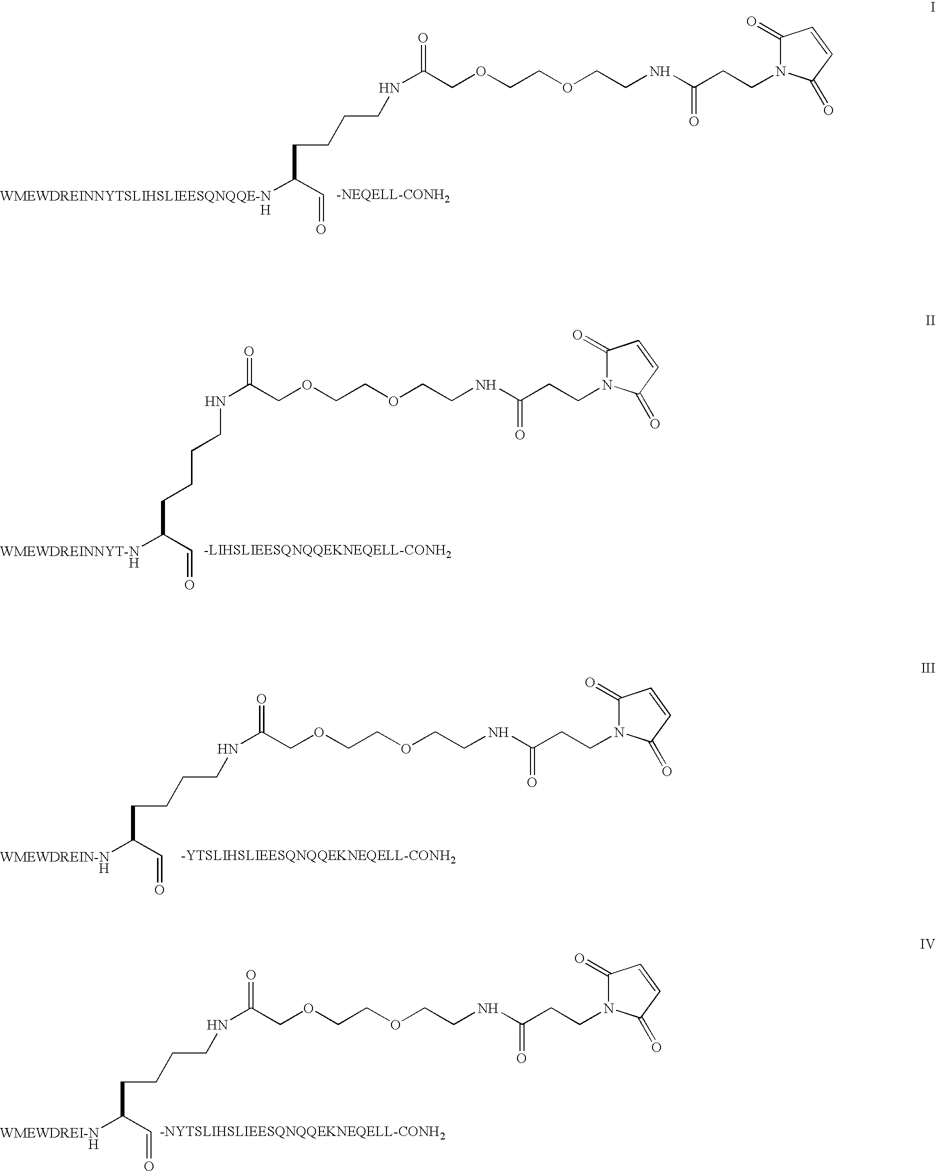 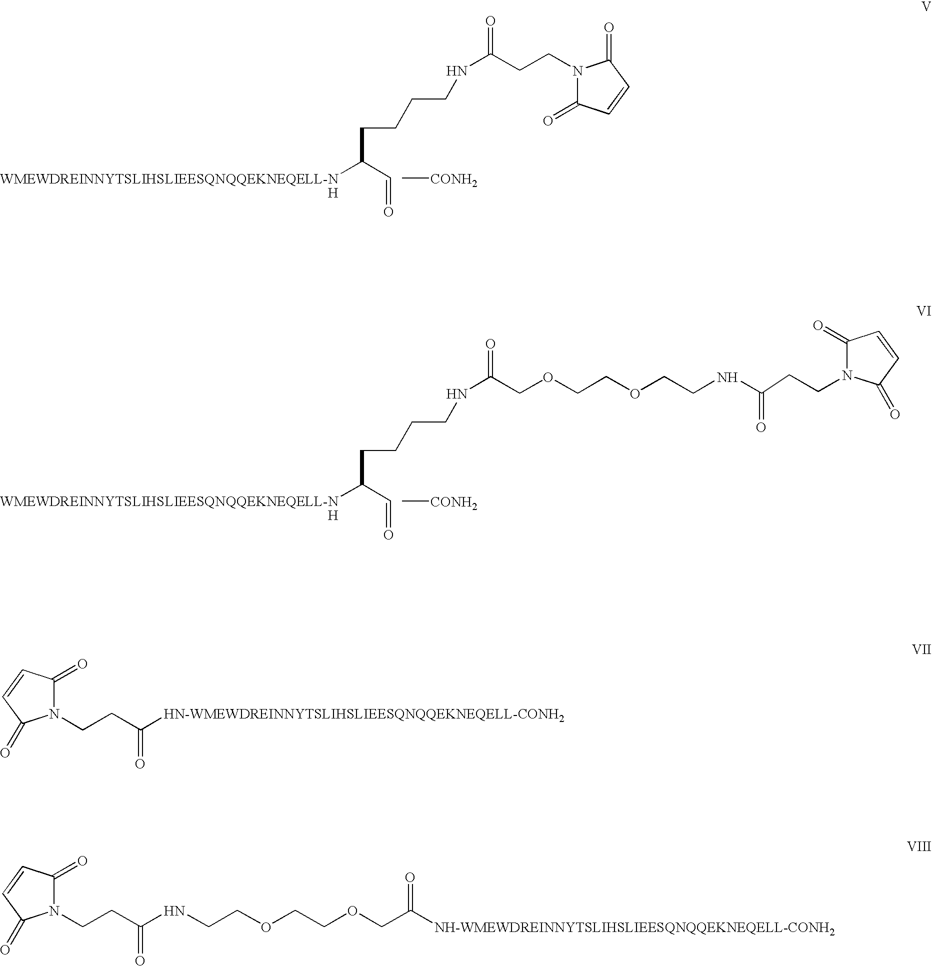
suitable for administering as an initial dose prior to viral infection or prior to the onset or recurrence of one or more symptoms associated with the infection.

22. The dosage formulation of claim 21 suitable for prophylactic use.

23. The dosage formulation of claim 21 suitable for therapeutic use.

24. The dosage formulation of claim 21, wherein the antifusogenic peptide is administered by subcutaneous injection or pulmonary inhalation.

25. The dosage formulation of claim 21, wherein the modified antifusogenic peptide is covalently coupled to a blood protein.

26. The dosage formulation of claim 21, wherein the blood protein is a recombinant protein.

27. The dosage formulation of claim 21, wherein the blood protein is serum albumin or recombinant albumin.

28. A method of reducing an infection from a virus selected from the group consisting of human immunodeficiency virus (HIV) infection, respiratory syncytial virus (RSV), human parainfluenza virus type 3 (HPIV-3), measles virus (MeV) and simian immunodeficiency virus (SIV) in a subject, comprising
administering one or more initial doses of a modified antifusogenic peptide, or a conjugate thereof, to a subject, prior to infection or prior to the onset or recurrence of one or more symptoms associated with the infection, thereby treating or preventing the infection, wherein the modified antifusogenic peptide is a compound selected from the group consisting of (SEQ ID NOS 2, 49-51, 13, 13, 2 and 2, respectively, in order of appearance): 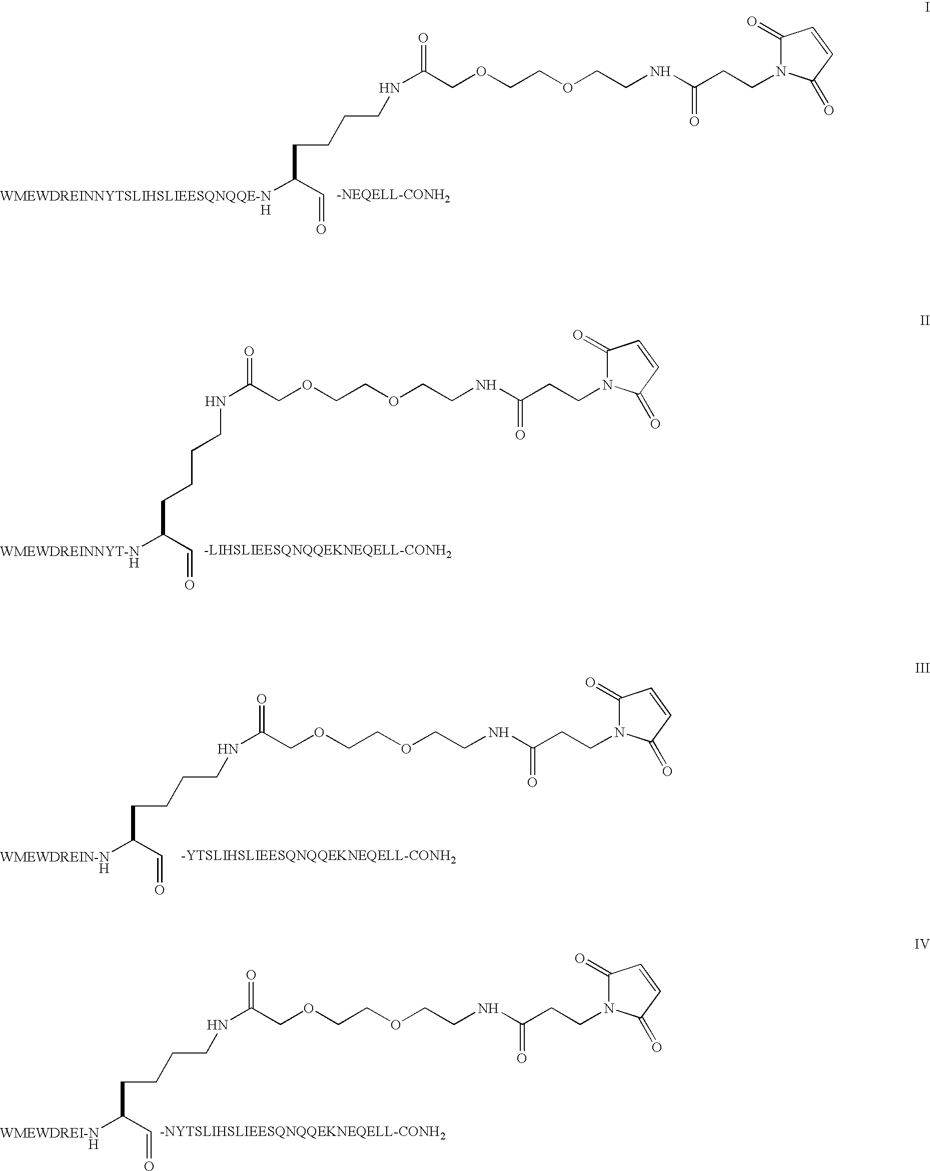 [chemical structure]
